(19) United States
(12) Patent Application Publication
(10) Pub. No.: US 2026/0185073 A1
(43) Pub. Date: Jul. 2, 2026

(54) ALTERED CYTIDINE DEAMINASES AND METHODS OF USE

(71) Applicant: ILLUMINA, INC., San Diego, CA (US)

(72) Inventors: Dewei Joel Toh, Singapore (SG); Lekha Ravichandraprabhu, Singapore (SG); Anna Traczyk, Singapore (SG); Eric Brustad, San Diego, CA (US); Shu Ting Tan, Singapore (SG); Rebekah Karadeema, San Diego, CA (US); Gaetano Speciale, San Diego, CA (US)

(73) Assignee: ILLUMINA, INC., San Diego, CA (US)

(21) Appl. No.: 19/422,853

(22) Filed: Dec. 17, 2025

Related U.S. Application Data
(63) Continuation of application No. PCT/US2024/049003, filed on Sep. 27, 2024.
(60) Provisional application No. 63/541,077, filed on Sep. 28, 2023.
(60) Provisional application No. 63/541,084, filed on Sep. 28, 2023.
(60) Provisional application No. 63/541,092, filed on Sep. 28, 2023.
(60) Provisional application No. 63/541,097, filed on Sep. 28, 2023.
(60) Provisional application No. 63/541,099, filed on Sep. 28, 2023.

(51) Int. Cl. C12N 9/78; C12Q 1/6806; C12Q 1/6827

(52) U.S. Cl. CPC C12N 9/78; C12Q 1/6806; C12Q 1/6827; C12Y 305/04005

(57) ABSTRACT

The present disclosure is concerned with modified proteins, methods, compositions, and kits for mapping of methylation status of nucleic acids, including 5-methylcytosine. In some embodiments, the proteins are altered cytidine deaminases, such as altered APOBEC3A proteins. In some embodiments, the proteins selectively act on cytosines and have significantly reduced activity on modified cytosines, such as 5-methylcytosine.

### CROSS-REFERENCE TO RELATED APPLICATIONS

This application is a continuation under 35 U.S.C. 111 (a) of International Application No. PCT/US2024/049003, filed Sep. 27, 2024, which claims the benefit of U.S. Provisional Application Ser. No. 63/541,077, filed on Sep. 28, 2023, U.S. Provisional Application Ser. No. 63/541,084, filed on Sep. 28, 2023, U.S. Provisional Application Ser. No. 63/541,092, filed on Sep. 28, 2023, U.S. Provisional Application Ser. No. 63/541,097, filed on Sep. 28, 2023, and U.S. Provisional Application Ser. No. 63/541,099, filed on Sep. 28, 2023. The disclosure of any priority document claimed is incorporated by reference herein in its entirety.

### SEQUENCE LISTING

This application contains a Sequence Listing electronically submitted via Patent Center to the United States Patent and Trademark Office as an XML file entitled “2673_US01.xml” having a size of 1,389,713 bytes and created on Mar. 13, 2026. The information contained in the Sequence Listing is incorporated by reference herein.

### FIELD

Embodiments of the present disclosure relate to modified proteins, methods, compositions, and kits for mapping of nucleotide methylation status.

### BACKGROUND

Modified DNA cytosines, including 5-methylcytosine (5mC) and 5-hydroxymethyl cytosine (5hmC), are a well-studied epigenetic modification that play fundamental roles in human development and disease. Its genome-wide distribution differs between tissue types, and between healthy and diseased states. In recent years, 5mC has also gained prominence as a tool for clinical diagnostics: its distribution in cell-free DNA (cfDNA)—obtained from a liquid biopsy—can be used for the tissue-specific prediction of early-stage cancer or monitoring of cancer recurrence or remission after treatment. As a result, there has been an intense focus on developing methods for mapping modified DNA cytosines at single base resolution, with minimal loss of sample DNA quantity, quality, and complexity. Current methods for mapping modified DNA cytosines, however, often exhibit limitations including (i) degradation of sample DNA due to prolonged chemical treatment at non-neutral pH and high temperatures, (ii) loss of sample DNA complexity due to conversion of unmethylated DNA bases to uracil, resulting in low complexity genome mapping, (iii) multi-step conversion, requiring both enzymes and chemical treatment, and (iv) for antibody-based 5mC detection, resolution of detection is limited to regions of approximately 150 nucleotides, precluding the identification of its exact location in the genome.

### SUMMARY OF THE APPLICATION

Provided herein are altered cytidine deaminases (ACDs) having C-selective deaminase activity and optionally including one or more stability-enhancing alteration, such as two or more stability-enhancing alterations, wherein conversion of cytosine (C) to uracil (U) by deamination occurs at a greater rate than conversion of 5-methyl cytosine (5mC) to thymidine (T) by deamination. The C-selective deaminase activity of the ACD can be the result of one or more substitution mutations, deletions, or insertions. For instance, a C-selective deaminase can include (a) a substitution mutation at position functionally equivalent to Tyrosine 130 (Tyr130, Y130), optionally wherein the substitution is L, F or I, preferably L; (b) a substitution mutation at position functionally equivalent to Tyrosine 132 (Tyr132, Y132), optionally wherein the substitution is H or P; (c) a substitution mutation at a position functionally equivalent to Aspartic Acid 133 (Asp133, D133), wherein the substitution is any amino acid except P or Y; or (d) a combination of (a), (b) and (c). In one embodiment, the substitution at D133 is W, D, K or R. In one embodiment, the one or more amino acid substitution mutations include a substitution at a position functionally equivalent to Tyr130 in a wild-type ACD, at a position functionally equivalent to Tyr132 in a wild-type ACD, or a combination thereof, wherein the wild-type ACD is a wild-type APOBEC3A (SEQ ID NO:3). In one embodiment, the at least one amino acid substitution mutation at the position functionally equivalent to Tyr130 is leucine (L), phenylalanine (F), or isoleucine (I). In some embodiments an ACD includes the substitution mutations Tyr130Leu/Tyr132His, Tyr130Phe/Tyr132Pro, Tyr132Pro, Tyr130Leu/Tyr132His/D133X, Tyr130Phe/Tyr132Pro/D133X, or Tyr132Pro/D133X, or Tyr132Ile/Tyr132Pro/D133X, wherein X is W, D, K, or R. In one embodiment, an ACD can include one or more alterations that enhance the selectivity of an ACD for C including (i) a substitution mutation at a position functionally equivalent to S103X, E138X, Q115X, R28X, Y136X, C108 X, or A112X, where X is an amino acid substitution different from the wild-type amino acid at that position, (ii) a deletion of amino acids at positions functionally equivalent to ΔI26-G27, where the position number designation is functionally equivalent to the position in a wild-type APOBEC3A, (iii) an insertion at positions functionally equivalent to Σ130-131:X, Σ132-133:X, Σ130-131:X, Σ131-132:X, Σ131-132:X, Σ102-103:X, Σ138-139:X, Σ137-138:X, or Σ134-135:X, where the position number designation is functionally equivalent to the position in a wild-type APOBEC3A (SEQ ID NO:3); or (iv) a combination of (i) and (ii), (ii) and (iii), (i) and (ii), or (i) and (ii) and (iii).

Optionally, an ACD can further include at least one stability-enhancing alteration, wherein the stability-enhancing alteration is a substitution mutation, a deletion, an insertion, or a combination thereof. In one embodiment, the one or more stability-enhancing alterations are selected from (a) one or more substitution mutations at one or more positions functionally equivalent to (a) A112X, A126X, A139X, A148X, A185X, A192X, A59X, A87X, C106X, C161X, C171X, C34X, D145X, D156X, D163X, D167X, D177X, D180X, D41X, D77X, D85X, E109X, E116X, E138X, E157X, E38X, G105X, G108X, G188X, G25X, G27X, H119X, H11X, H16X, H182X, H29X, H51LX, I17X, I26X, I89X, K47X, K60X, L135X, L62X, L78X, M14X, M48X, N117X, N196X, N21X, N42X, P80X, Q115X, Q141X, Q169X, Q184X, R111X, R123X, R189X, R39X, R74X, R91X, S103X, S183X, S187X, S20X, S45X, T118X, T164X, T19X, T31X, T93X, V110X, V79X, L12X, D14X, T19X, G27X, R28X, E38X, F54X, H56X, N57X, Y67X, L73X, D77X, S81X, Y90X, I96X, S97X, C101X, F102X, W104X, C106X, A107X, L114X, V120X, L122X, R128X, Y136X, M142X, A146X, K159X, C161X, L186X, (b) a deletion selected from ΔM1-L12, ΔE36-G53, ΔN61-G68, ΔW104-G105, ΔQ195-N199, or ΔI26-G27, and (c) combinations thereof, wherein the position number designation is functionally equivalent to the position in a wild-type APOBEC3A (SEQ ID NO:3) and X is an amino acid substitution different from the wild-type amino acid at that position. The one or more stability-enhancing alterations can include at least one substitution and at least one deletion, wherein the substitution mutation is at a position functionally equivalent to a stability-enhancing mutation selected from Table 4, and the deletion is a deletion selected from Table 6.

### BRIEF DESCRIPTION OF THE FIGURES

The following detailed description of illustrative embodiments of the present disclosure may be best understood when read in conjunction with the following drawings.

FIG. 1A-C shows the deamination scope of APOBEC3A. Cytosine (C), 5-methylcytosine (5mC), and 5-hydroxymethylcytosine (5hmC) nucleobases in single-stranded DNA are well characterized substrates of APOBEC3A. FIG. 1A shows conversion of C to uracil (U) by APOBEC3A. FIG. 1B shows conversion of 5mC to thymidine (T) by APOBEC3A. FIG. 1C shows conversion of 5hmC to 5-hydroxymethyl uracil (5hmU) by APOBEC3A. “˜” denotes the connection of the nucleobases to a single-stranded DNA molecule.

FIG. 1D and FIG. 1E show the result of treating a DNA sample with a wild-type APOBEC3A enzyme (FIG. 1D), and an example of one-step detection of unmodified cytosines using an altered cytidine deaminase described herein (FIG. 1E). The top strand of FIG. 1D-E shows C, 5mC, and 5hmC bases, and the bottom strand of FIG. 1D-E underlines the changed bases. 5mC nucleobases are marked with CH₃, 5hmC nucleobases are marked with CH₂—OH and uracil nucleobases are designated with capital “U.”

FIG. 2 is a schematic showing alignment of cytidine deaminase amino acid sequences using the Clustal O algorithm. An “*” (asterisk) indicates positions which have a single, fully conserved residue between some cytidine deaminases. A “:” (colon) indicates conservation between groups of strongly similar properties as below-roughly equivalent to scoring >0.5 in the Gonnet PAM 250 matrix. A “.” (period) indicates conservation between groups of weakly similar properties as below-roughly equivalent to scoring=<0.5 and >0 in the Gonnet PAM 250 matrix. The amino acids marked with “\hat{ }” show the ZDD motif SEQ ID NO: 12 (e.g., above amino acids 70 to 106 of sp|P31941|1-199). The amino acids marked with “\hat{ }” and “#” show the ZDD motif SEQ ID NO:13 (e.g., above amino acids 70 to 153 of sp|P31941|1-199). sp|P31941|1-199 is a human APOBEC3A, SEQ ID NO:3; XP_045219544.1 is an APOBEC3A from Macaca fascicularis, SEQ ID NO:19; AER45717.1 is an APOBEC3A from Pongo pygmaeus, SEQ ID NO:20; XP_003264816.1 is an APOBEC3A from Nomascus leucogenys, SEQ ID NO:21; PNI48846.1 is an APOBEC3A from Pan troglodytes, SEQ ID NO:22; and ADO85886.1 is an APOBEC3A from Gorilla gorilla, SEQ ID NO:23.

FIG. 3 shows deaminase activity of wild type (NEB APOBEC) and mutant APOBEC variants on substrates including unmodified C, 5mC, and 5hmC. Percent deamination values were determined from a SwaI restriction enzyme assay and quantified (see Example 1 of International Application Publication WO 2023/196572 A1). C deamination activity was measured in two independent experiments corresponding to the left and right panels.

FIG. 4 shows point mutations that have been tested at each amino acid position of an APOBEC3A (SEQ ID NO:3). Stabilizing mutations (black) denote variants which passed an initial screen to suggest stabilization over the parent construct (representing 20% of all variants tested).

FIG. 5A-5B show altered cytidine deaminases containing single point mutations in a Y130A/Y132H/C171A background. Proteins were expressed, purified and assessed for stability compared to the parent construct using either a (FIG. 5A) fluorimetry based assay to measure the Tm; or (FIG. 5B) LC/MS based activity assay performed at various temperatures. The optimal reaction temperature is shifted for stabilizing mutations compared to the parent construct, with higher 5mC conversion achieved at higher temperatures.

FIG. 6A-6B show altered cytidine deaminases containing mutations in a Y130A/Y132H/D133W/R74L/T19Y/C171A (ScE, SEQ ID NO:72) background that were expressed, purified and screened for stability. (FIG. 6A) An LC/MS assay was used to measure the fraction 5mC converted in a DNA oligo after incubation at 45° C. in the presence of denaturants (20% DMSO, 2 M betaine). Variants with a higher 5mC conversion than the parent construct (black) were moved forward to screening in a sequencing-based assay. (FIG. 6B) Temperature profile using a sequencing-based assay. 200 nM enzyme was incubated with substrate for 60 minutes at various temperatures. The optimal reaction temperature is shifted for stabilizing mutations, with higher 5mC conversion achieved at higher temperatures. All data points represent the mean of duplicate measurements.

FIG. 7 shows melting temperatures for selected constructs. C171A and T19Y confer higher stability in two different backgrounds compared to the parent construct. Stability improvements are additive as mutations are stacked in a Y130A/Y132H/D133W background.

FIG. 8 shows a temperature profile using a sequencing-based assay. The optimal reaction temperature is shifted for stabilizing mutations, with higher 5mC conversion achieved at higher temperatures.

FIG. 9 shows that Y132H enhances the substrate preference of D133W/Y130L towards unmodified C compared to tyrosine (Y) at position 132. Graph showing deamination activities of an ACD enzyme with substitution mutations at Y130 mutated to all 20 amino acids in combination with D133W substitution mutation either with Y132Y (wildtype amino acid) or Y132H substitution mutation.

FIG. 10A-10C shows the deamination activities of ACDs that have C-preferring properties. (FIG. 10A) Graph showing deamination activities of an ACD enzyme with substitution mutations at Y130 mutated to all 20 amino acids in combination with D133W substitution mutation either with Y132Y (wildtype amino acid) or Y132P substitution mutation. (FIG. 10B) Graph showing Y132H/Y130L/D133X using kingfisher eluate. Y-axis represents % C deamination and X-axis represents % 5mC deamination. Letters in the Y130L box represent respective D133X mutants. Red data points are ScF mutant ACD (SEQ ID NO:74) as positive controls that serve as a proxy for 5mC selective activity. (FIG. 10C) Same data points in (FIG. 10B) represented in the form of bar graph. Y-axis is the % deamination of C (lighter color) and 5mC (darker color). With Y130L mutation present in the APOBEC mutants, C deamination is largely preferred over 5mC, represented by the taller yellow (or lighter colored) bars compared to blue (or darker colored) bars.

FIG. 11A-11B shows that certain substitution mutations prefer unmodified C over 5mC.

FIGS. 12A, 12B, and 12C show three different 5mC mapping workflows using bisulfite, EM-Seq (NEB), and ACDs described herein.

FIG. 13 shows substrate selectivity curves of NEB APOBEC and 5mC-selective APOBEC3A mutants. Various 5mC and C % deamination data points of NEB APOBEC and engineered 5mC-selective APOBECs were plotted (X-axis represents 5mC deamination (%); Y-axis represents C deamination (%)). Data points were generated by subjecting the APOBECs to various deamination conditions, such as protein amounts and time points. Scatter plot made up of the data points of respective proteins can be traced forming the selectivity curves. The more the selectivity curve is skewed towards the X-axis, the more 5mC selective it is.

FIG. 14 shows the architecture of engineered A3A dimers. Several homodimeric and heterodimeric constructs were designed. Two engineered APOBEC3A (A3A) were fused together by a 32 amino acids peptide linker. The homodimers included Y130A/Y132H/D133W, with or without R74L stability mutations. There were 4 heterodimers made up of engineered A3A (Y130A/Y132H/D133W) with and without R74L at the N or C-terminal, and A3A (E72A) at the N or C-terminal. All proteins were expressed with a C terminal His tag.

FIG. 15A-15D shows engineered A3A dimers that show significant improvement of activity at higher reaction temperature. (FIG. 15A) Deamination activity of each enzyme (0.7 μM) was evaluated in NGS-based quantitative deamination assay at various reaction time and temperature (25, 37, 42 and 50° C.). (FIG. 15B) Fusing the mutants with a linker did not affect their selectivity. Data points show deaminated C (%) plotted against deaminated 5mC (%) generated by the monomers, heterodimers and homodimers. Trendlines generated at respective deamination incubation temperatures (25° C., 37° C., 42° C. and 50° C.) tested were continuous, indicating that the dimers exhibited selectivity comparable to monomeric constructs. (FIG. 15C). At 42° C., 5mC deamination was favored (selectivity curve leaning towards the right and X-axis), compared to 25° C. and 37° C. (FIG. 15D) The engineered homodimer was found to be more active and thermal stable than the monomer. Both monomers and dimers were purified by Ni-NTA affinity, heparin-affinity chromatography and desalted. The heparin purification step resulted in faster enzyme kinetics at 37° C. in both monomer and dimer, but the high reactivity at higher temperature is not observed for the monomer. At 50° C., the monomer had almost no activity, whereas the dimer was able to deaminate up to ˜50% of mC within 15 minutes at 50° C. All these improved performances of the dimer were not observed in monomer even when double amount of monomer is added to the reaction (0.2 μM and 0.4 μM).

FIG. 16A-16C shows that the R74L/Y130A/Y132H/D133W dimer has improved stability at temperatures of 40° C. to 50° C. (FIG. 16A). At the Y-axis, 5mC to T conversion level in the fully-CpG methylated pUC19 and C to U conversion in non-methylated lambda DNA indicate the true positive and false positive conversion, respectively. On the NaOH-treated library, the dimer could withstand a higher reaction temperature of up to 42° C. without showing reduced enzyme activity, whereas the activity of monomer declined at temperatures above 38° C. When the monomer was supplemented with 10 μM ZnCl2, higher reaction temperatures were tolerated, but the effect was not as substantial as in the dimer. For libraries denatured in DMSO, dimer constructs were able to react at temperatures up to 38° C. without showing reduced enzyme activity, whereas the activity of the monomer declined at temperatures above 33° C. This demonstrated that dimerization enabled higher reaction temperatures in multiple reaction formulations. (FIG. 16B) 5mC to T conversion level in both methylated DNA and C to U conversion level in non-methylated DNA were normalized to 1 by its maximum level for in order to compare how temperature affects conversion in methylated and unmethylated DNA. Smooth (CpG_Beta_pUC Normalized) is the top trace, Smooth (CpG_Beta_lambda Normalized) is the bottom trace. (FIG. 16C) Monomer and homodimer ScD were incubated with the denatured library at various temperatures, the resulted data points were fitted to polynomial regression, and the maximum point of the fitted curve is considered as the temperature at which the enzyme has the highest level of 5mC or C deamination (Tmax). Tmax values were elevated for the monomer with 10 μM Zn added and were even higher for the homodimer.

FIG. 17 shows that the dimer has improved reaction kinetics compared to monomer. Variation of initial velocity (y-axis) of the reaction at varying amount of DNA substrate (x-axis). Deamination reactions were performed at 45° C., using 1 μM of monomer and 0.5 μM of dimer, to achieve the same equivalent of active sites added in the reaction. Linear rates were measured by incubation of the reactions at 5 nM, 10 nM, 20 nM, 40 nM, 80 nM, 120 nM, 160 nM, 200 nM of DNA substrate for 2 minutes, 5 minutes, 10 minutes, and 15 minutes, followed by heat inactivation at 95° C. for 5 minutes. The reactions were sequenced, and the percentage of conversion converted to nM of product generated, and fitted to linear regression to obtain the velocity.

FIG. 18A to 18H show amino acid sequences of SEQ ID NOs: 16, 17, 37-56, 68-79.

Schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION OF EXEMPLARY EMBODIMENTS

Terms used herein will be understood to take on their ordinary meaning in the relevant art unless specified otherwise. Several terms used herein and their meanings are set forth below.

Terms used herein will be understood to take on their ordinary meaning in the relevant art unless specified otherwise. Several terms used herein and their meanings are set forth below.

As used herein, the terms “organism,” “subject,” are used interchangeably and refer to microbes (e.g., prokaryotic or eukaryotic) animals and plants. An example of an animal is a mammal, such as a human.

As used herein, the term “target nucleic acid,” is intended as a semantic identifier for the nucleic acid in the context of a method or composition or kit set forth herein and does not necessarily limit the structure or function of the nucleic acid beyond what is otherwise explicitly indicated. Reference to a nucleic acid such as a target nucleic acid includes both single-stranded and double-stranded nucleic acids, and both DNA and RNA, unless indicated otherwise. The term library refers to the collection of target nucleic acids containing known common sequences, such as a universal sequence or adapter, at their 3′ and 5′ ends.

As used herein, the term “adapter” and its derivatives, e.g., universal adapter, refers generally to any linear oligonucleotide which can be attached to a target nucleic acid. An adapter can be single-stranded or double-stranded DNA, or can include both double-stranded and single-stranded regions. An adapter can include a universal sequence that is substantially identical, or substantially complementary, to at least a portion of a primer, for example a universal primer; an index (also referred to herein as a barcode or tag) to assist with downstream error correction, identification, or sequencing; and/or a unique molecular identifier. In some embodiments, the adapter is substantially non-complementary to the 3′ end or the 5′ end of any target sequence present in the sample. In some embodiments, suitable adapter lengths are in the range of about 6-100 nucleotides, about 12-60 nucleotides, or about 15-50 nucleotides in length. For instance, the terms “adaptor” and “adapter” are used interchangeably.

As used herein, the term “universal,” when used to describe a nucleotide sequence, refers to a region of sequence that is common to two or more nucleic acid molecules where the molecules also have regions of sequence that differ from each other. A universal sequence that is present in different members of a collection of nucleic acids can be used as, for instance, a “landing pad” in a subsequent step to anneal a nucleotide sequence that can be used as a primer for addition of another nucleotide sequence, such as an index, to a target nucleic acid. A universal sequence that is present in different members of a collection of nucleic acids can allow capture of multiple different nucleic acids using a population of universal capture nucleic acids, e.g., capture oligonucleotides that are complementary to a portion of the universal sequence, e.g., a universal capture sequence. Non-limiting examples of universal capture sequences include sequences that are identical to or complementary to P5 and P7 primers. Similarly, a universal sequence present in different members of a collection of molecules can allow the replication (e.g., sequencing) or amplification of multiple different nucleic acids using a population of universal primers that are complementary to a portion of the universal sequence, e.g., a universal anchor sequence. In one embodiment universal anchor sequences are used as a site to which a universal primer (e.g., a sequencing primer for read 1 or read 2) anneals for sequencing. A capture oligonucleotide or a universal primer therefore includes a sequence that can hybridize specifically to a universal sequence.

The terms “P5” and “P7” may be used when referring to a universal capture sequence or a capture oligonucleotide. The terms “P5′” (P5 prime) and “P7′” (P7 prime) refer to the complement of P5 and P7, respectively. It will be understood that any suitable universal capture sequence or a capture oligonucleotide can be used in the methods presented herein, and that the use of P5 and P7 are exemplary embodiments only. Uses of capture oligonucleotides such as P5 and P7 or their complements on flow cells are known in the art, as exemplified by the disclosures of WO 2007/010251, WO 2006/064199, WO 2005/065814, WO 2015/106941, WO 1998/044151, and WO 2000/018957, which are incorporated by reference as to P5 and P7 and their uses. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, the term “primer” and its derivatives refer generally to any nucleic acid that can hybridize to a target sequence of interest. Typically, the primer functions as a substrate onto which nucleotides can be polymerized by a polymerase or to which a polynucleotide can be ligated; in some embodiments, however, the primer can become incorporated into the synthesized nucleic acid strand and provide a site to which another primer can hybridize to prime synthesis of a new strand that is complementary to the synthesized nucleic acid molecule. In some embodiments, the primer can be used for hybridization to a predetermined sequence, for instance a predetermined sequence that includes one or more nucleotides that identify the location of a modified cytosine. In one embodiment, a “primer” includes a sequence present in a guide RNA used with a CRISPR-based system to hybridize to a predetermined sequence. The primer can include any combination of nucleotides or analogs thereof. In some embodiments, the primer is a single-stranded oligonucleotide or polynucleotide.

The terms “polynucleotide” and “oligonucleotide” and “nucleic acid” are used interchangeably herein to refer to a polymeric form of nucleotides of any length, and may include ribonucleotides, deoxyribonucleotides, analogs thereof, or mixtures thereof. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, that is complementary or copy DNA produced from a RNA template, for example by the action of reverse transcriptase.

As used herein, an “index” (also referred to as an “index region,” “index adaptor,” “tag,” or a “barcode”) refers to a unique nucleic acid tag that can be used to identify a sample or source of the nucleic acid material, or a compartment in which a target nucleic acid was present. The index can be present in solution or on a solid-support, or attached to or associated with a solid-support and released in solution or compartment. When nucleic acid samples are derived from multiple sources, the nucleic acids in each nucleic acid sample can be tagged with different nucleic acid tags such that the source of the sample can be identified. Any suitable index or set of indexes can be used, as known in the art and as exemplified by the disclosures of U.S. Pat. No. 8,053,192, PCT Publication No. WO 05/068656, and U.S. Pat. Publication No. 2013/0274117. In some embodiments, an index can include a six-base Index 1 (i7) sequence, an eight-base Index 1 (i7) sequence, an eight-base Index 2 (i5e) sequence, a ten-base Index 1 (i7) sequence, or a ten-base Index 2 (15) sequence from Illumina, Inc. (San Diego, CA).

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g., a PCR product) or multiple copies of the nucleotide sequence (e.g., a concatemeric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, “amplify”, “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification is typically the exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

As used herein, the term “polymerase chain reaction” (“PCR”) refers to the method of Mullis U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as PCR. Because the desired amplified segments of the polynucleotide of interest become the predominant nucleic acid sequences (in terms of concentration) in the mixture, they are said to be “PCR amplified”. In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

As used herein, “amplification conditions” and its derivatives, generally refers to conditions suitable for amplifying one or more nucleic acid sequences. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences flanked by a universal sequence, or target specific primers, or to amplify an amplified target sequence flanked by one or more adapters. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending and separating are repeated. Typically, the amplification conditions include cations such as Mg²⁺ or Mn²⁺ and can also include various modifiers of ionic strength.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher. It is also possible to detect the amplified target sequences by several different methodologies (e.g., gel electrophoresis followed by densitometry, quantitation with a bioanalyzer or quantitative PCR, hybridization with a labeled probe; incorporation of biotinylated primers followed by avidin-enzyme conjugate detection; incorporation of 32p-labeled deoxynucleotide triphosphates into the amplified target sequence).

As used herein, the term “amplification site” refers to a site in or on an array where one or more amplicons can be generated. An amplification site can be further configured to contain, hold or attach at least one amplicon that is generated at the site.

As used herein, the term “array,” “analyte array,” and “microarray” are used interchangeably and refer to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, droplets, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “compartment” is intended to mean an area or volume that separates or isolates something from other things. Exemplary compartments include, but are not limited to, vials, tubes, wells, droplets, boluses, beads, vessels, surface features, flow cell, or areas or volumes separated by physical forces such as fluid flow, magnetism, electrical current or the like. In one embodiment, a compartment is a well of a multi-well plate, such as a 96- or 384-well plate. As used herein, a droplet may include a hydrogel bead, which is a bead for encapsulating one or more nuclei or cell, and includes a hydrogel composition. In some embodiments, the droplet is a homogeneous droplet of hydrogel material or is a hollow droplet having a polymer hydrogel shell. Whether homogenous or hollow, a droplet may be capable of encapsulating one or more nuclei or cells. In some embodiments, the droplet is a surfactant stabilized droplet. In some embodiments, a single cell or Nuclei is present per compartment. In some embodiments, two or more cells or Nuclei are present per compartment. In some embodiments, each compartment contains a compartment-specific index. In some embodiments, the index is in solution or attached or associated with a solid-phase in each compartment.

The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

As used herein, the term “clonal population” refers to a population of nucleic acids that is homogeneous with respect to a particular nucleotide sequence. The homogenous sequence is typically at least 10 nucleotides long, but can be even longer including for example, at least 50, 100, 250, 500 or 1000 nucleotides long. A clonal population can be derived from a single target nucleic acid or template nucleic acid. Typically, all of the nucleic acids in a clonal population will have the same nucleotide sequence. It will be understood that a small number of mutations (e.g., due to amplification artifacts) can occur in a clonal population without departing from clonality.

As used herein, a “pattern of cytosine modification,” also referred to as a “methylation profile,” refers to the pattern with which both methylation and unmethylation of cysteines is distributed in the genome of a cell or an organism. A “pattern” is inclusive of both modified cytosines and non-modified cytosines. The pattern can be defined in several distribution dimensions: by organ, by tissue, by status of disease or pathological condition (e.g., cancer, neurophysiological), by genome segment (e.g., chromosome or genetic coordinates on a chromosome), by gene, by CpG island, a group of cytosines, or by the site of a modified cytosine. A pattern of cytosine modification can have a known correlation with a disease or pathological condition, or correlation of a pattern of cytosine modification with a disease or pathological condition can be identified using methods described herein. A pattern of cytosine modification can be present at a specific locus (e.g., location) in a genome, and that specific location can be a single modified cytosine or a set of modified cytosines, e.g., a CpG island. A pattern of cytosine modification can be identified by using a predetermined sequence, e.g., a method of using an altered cytidine deaminase can be designed and practiced with the intent of determining a pattern of cytosine modification, for instance, the methylation status of one of more specific cytosines, the methylation status of one or more specific cytosines present at a specific location of a genome, or the combination thereof.

As used herein, the term “each,” when used in reference to a collection of items, is intended to identify an individual item in the collection but does not necessarily refer to every item in the collection unless the context clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Conditions that are “suitable” for an event to occur, such as converting 5 methylcytosine to thymidine by deamination, or “suitable” conditions are conditions that do not prevent such events from occurring. Thus, these conditions permit, enhance, facilitate, and/or are conducive to the event.

As used herein, “providing” in the context of a protein, sample of DNA or RNA, or composition means making the protein, sample of DNA or RNA, or composition, purchasing the protein, sample of DNA or RNA, or composition, or otherwise obtaining the protein, sample of DNA or RNA, or composition.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

While polynucleotide sequences encoding an altered cytidine deaminase are described herein as DNA sequences, it is understood that the complements, reverse sequences, and reverse complements of the DNA sequences can be easily determined by the skilled person. It is also understood that the sequences described herein as DNA sequences can be converted from a DNA sequence to an RNA sequence by replacing each thymidine nucleotide with a uracil nucleotide.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 4.5, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

### Altered Cytidine Deaminases (ACDs)

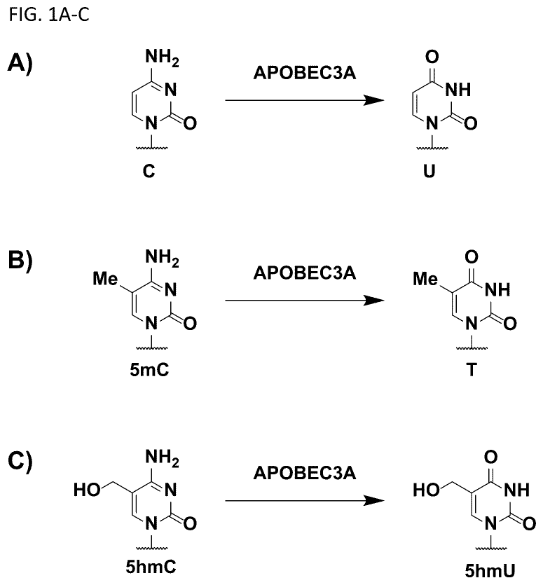

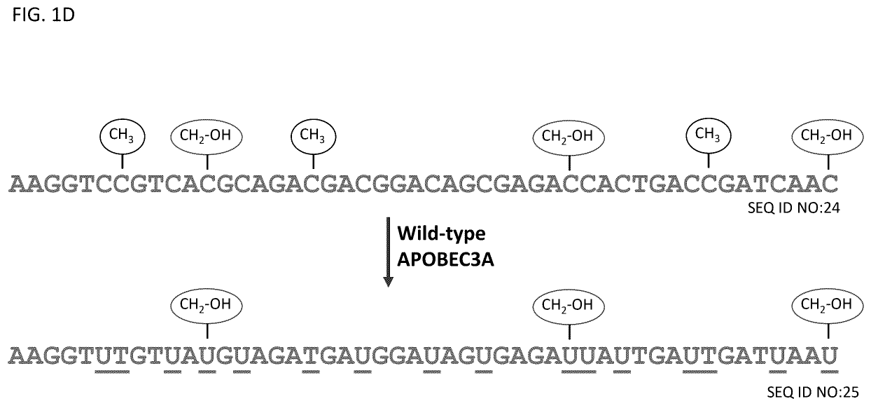

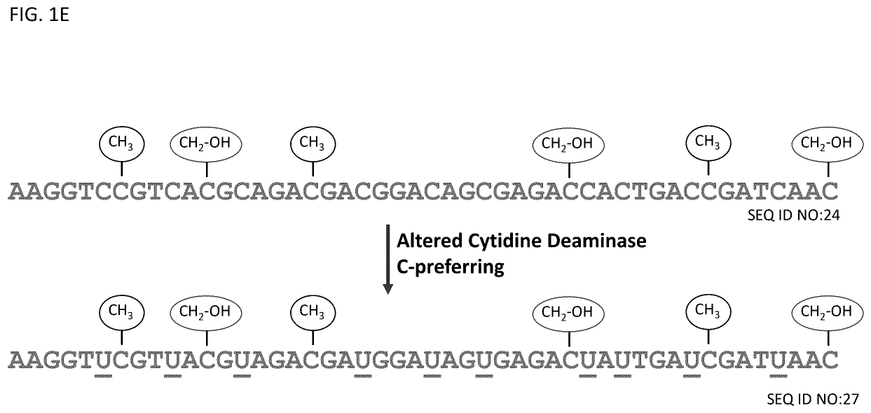

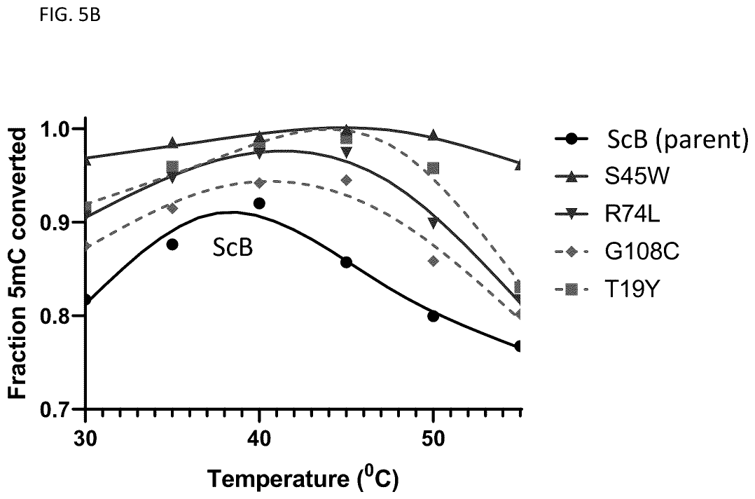

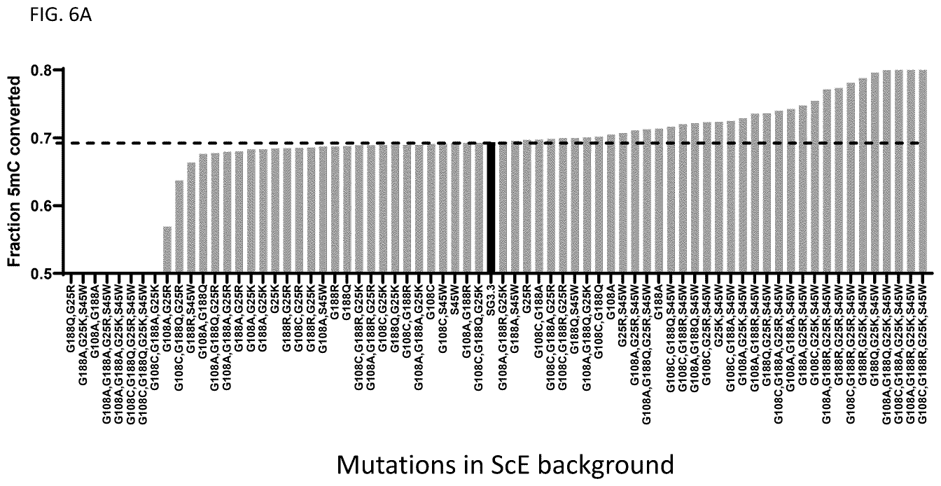

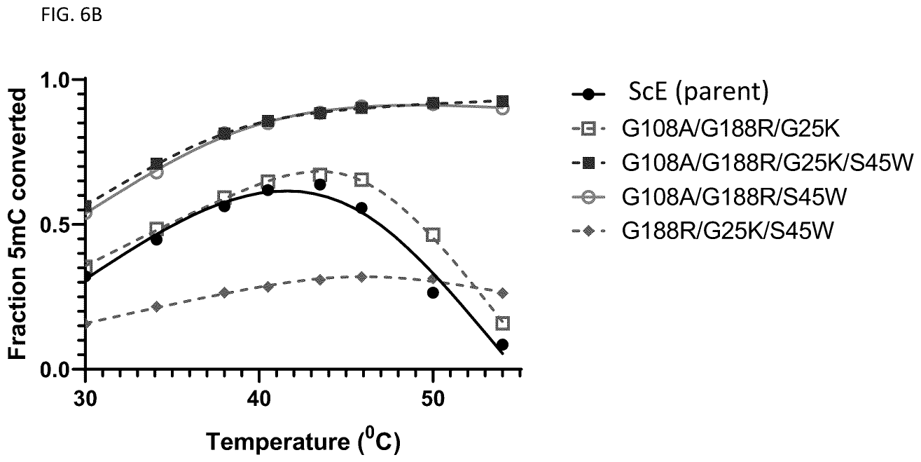

Wild type APOBEC3A deaminates cytosine (C), 5 methyl cytosine (5mC), and 5-hydroxymethyl cytosine (5hmC) efficiently in single-stranded DNA (FIG. 1A-C). Treatment of DNA, such as genomic DNA, with wild type APOBEC3A results in the conversion of C to uracil (U), 5mC to thymidine (T), and 5hmC to 5-hydroxymethyluracil (5hmU) and reduces the complexity of the DNA for sequencing (FIG. 1D). Point mutations in human APOBEC3A proteins were produced in previous analyses and the ability of the mutant APOBEC3A proteins to convert cytosine to uracil were determined. Modification of the tyrosine residue at position 130 to alanine (Y130A) consistently resulted in an APOBEC protein with no activity (see FIG. 6c of Bulliard et al., 2011, J Virol., 85 (4): 1765-1776, and FIG. 5a of Shi et al., 2017, Nat Struct Mol Biol., 24 (2): 131-139). Proceeding contrary to Bulliard and Shi, it was discovered that certain mutations at position 130 of APOBEC3A alter the enzyme's rate of deamination on 5mC compared to C substrates (International Application Publication WO 2023/196572).

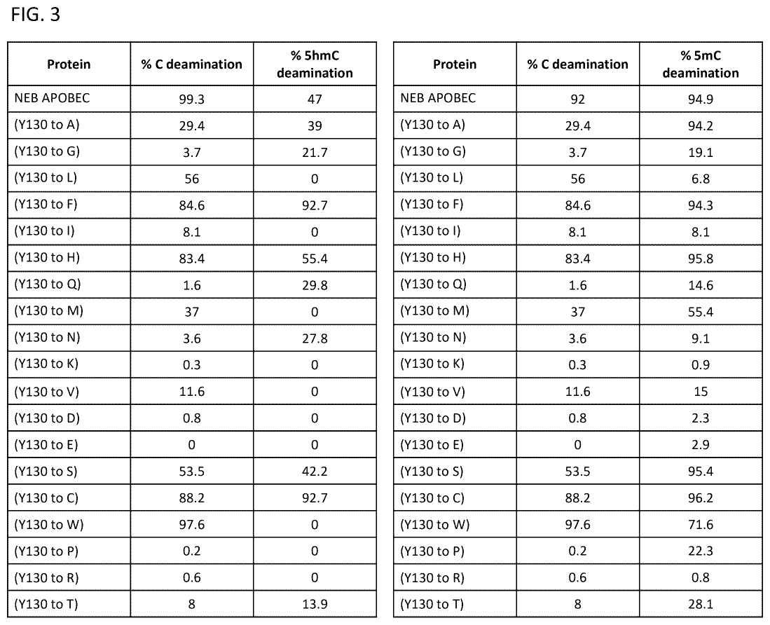

The cognate tyrosine (Y) at position 130 was individually mutated to all possible canonical amino acid substitutions, including A, C, D, E, F, G, H, I, K, L, M, N, P, Q, R, S, T, V, and W, and evaluated for activity on C and 5mC, and 5hmC substrates (FIG. 3). An APOBEC3A mutant containing a tyrosine to leucine point mutation at position 130 (Y130L) was found to preferentially deaminate C instead of 5mC and 5hmC (C was converted to U at a greater rate than 5mC was converted to T and 5hmC was converted to 5hmU). As a result, the treatment of DNA with an ACD of the present disclosure preferentially converts C to uracil (FIG. 1E). Analysis of the sample DNA after treatment with modified cytidine deaminase described herein, for example, by sequencing of the sample DNA, and optional comparison to a reference (e.g., reference sequence) permits easy identification of C to T point mutations, and these point mutations are inferred as positions having a C. The other cytosines in the sequence that remain unchanged are inferred as positions having a methylated C, such as 5mC or 5hmC.

As further described herein, a second mutation at Y132 increased the activity of an APOBEC3A mutant containing Y130L (and, as described herein, Y130F) to preferentially deaminate C instead of 5mC compared to the same APOBEC protein containing one substitution mutation at Y130. In one embodiment, the substitution mutation at the second position is histidine (H) or proline (P), such as a tyrosine to histidine point mutation at position 132 (Y132H) or tyrosine to proline point mutation at position 132 (Y132P).

The inventors have discovered that the selectivity of APOBEC3A mutants containing substitution mutations that preferentially deaminate C over 5mC can be further modified to enhance the selectivity of the enzyme. Modification of the aspartic acid residue at position 133 to tryptophan (D133W) in an ACD with enhanced selectivity for C resulted in an ACD with further enhancement of the selectivity for C compared to the same protein without the D133W mutation.

The introduction of a selectivity-enhancing mutation like D133W was found to reduce the thermal melting point of ACDs that also included a substitution mutation at Y130, at Y132, or at both Y130 and Y132. The inventors have discovered that modification of certain amino acids increased stability of APOBEC proteins. Examples of these stability-enhancing modifications are described in Table 4-8. For instance, modification of the arginine residue at position 74 to leucine (R74L) in an ACD containing the Y130L/Y132H/D133W substitution mutations, increased stability compared to the same protein without the R74L substitution mutation, and retained the enhanced selectivity for C conferred by the D133W substitution mutation. Selectivity-enhancing mutations (e.g., D133W and other selectivity-enhancing alterations) and stability-enhancing mutations (e.g., R74L and other stability-enhancing alterations) are described in detail herein and can be combined to provide selectivity-enhanced cytosine deaminases.

Provided herein are altered cytidine deaminases (ACDs, or the singular form ACD), compositions including an ACD, methods of using an ACD, and kits that include an ACD. In some embodiments, the ACDs are used in one-step, enzymatic methods for mapping modified cytosines, such as 5mC and 5hmC, at single base resolution. The examples provided herein describe ACDs based on APOBEC3A, and it is expected that other APOBEC proteins, modified as described herein, can be used.

A cytidine deaminase is considered to be an altered cytidine deaminase (ACD) if it has the activity of deaminating C and includes at least one of the substitution mutations described herein. An altered cytidine deaminase that preferentially deaminates C instead of 5mC (converts C to U at a greater rate than converting 5mC to T) compared to the equivalent wild-type enzyme is referred to herein interchangeably as “C-enhanced activity,” “C-preferring activity,” “C-selective activity” or having “5mC-defective deaminase activity.”

ACDs include apolipoprotein B mRNA editing enzymes, catalytic polypeptide-like (APOBEC) and activation induced cytidine deaminase (AID). Wild-type APOBEC and AID cytidine deaminases have the activity of deaminating cytidine I of DNA and/or RNA to form uridine (U). An ACD of the present disclosure has an altered rate of deamination of C, 5mC, and/or 5hmC when compared to the wild-type enzyme. A cytidine deaminase of the present disclosure can be referred to herein as an “altered cytidine deaminase,” “recombinant cytidine deaminase,” “ACD,” “recombinant ACD,” “mutant cytidine deaminase,” or “modified cytidine deaminase” and refers to any of the engineered ACDs described herein that include one or more changes from a reference (i.e., wild-type) amino acid sequence that provide one or more of the activities described herein, including but not limited to an altered deamination profile, e.g., alters its ability to preferentially deaminate one form of cytosine over another, enhanced selectivity for C, and/or enhanced stability.

Whether a protein has cytidine deaminase activity may be determined by in vitro assays. On example of an in vitro assay is based on digestion with the restriction enzyme SwaI (see Example 1 of International Application Publication WO 2023/196572). A protein that can deaminate C to U has cytidine deaminase activity.

An ACD that preferentially deaminates C instead of 5mC (i.e., has C-selective deaminase activity) can have a catalytic efficiency that is at least 10-fold, at least 20-fold, at least 30-fold, at least 40-fold, at least 50-fold, or at least 100-fold higher on C than 5mC substrates. In some embodiments, the C-selective ACD has at least 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 320-fold, 340-fold, 360-fold, 380-fold, 400-fold, 425-fold, 450-fold, 475-fold, 500-fold, 525-fold, 550-fold, 575-fold, 600-fold, 700-fold, 800-fold, 900-fold, 1000-fold higher activity, including ranges and amounts in between. In one embodiment, an ACD that preferentially deaminates C instead of 5mC can have a catalytic efficiency that is no greater than 1500-fold higher on C than 5mC substrates.

In certain embodiments, an ACD of the present disclosure is based on a member of the APOBEC protein family. An ACD of the present disclosure that is “based on” a member of the APOBEC protein family means the ACD is an APOBEC protein that includes one or more of the substitution mutations described herein as compared to a reference APOBEC sequence. An ACD of the present disclosure that is “based on” a member of the APOBEC protein family can also include conservative and/or nonconservative mutations as described herein. The positions of the alterations, substitutions or deletions will be at functionally equivalent amino acids from the APOBEC A3 reference sequence, as described herein.

The APOBEC protein family includes subfamilies AID, APOBEC1, APOBEC2, APOBEC3 (including 3A, 3B, 3C, 3D, 3F, 3G, 3H), and APOBEC4. An ACD of the present disclosure can be based on a member of the AID subfamily, the APOBEC1 subfamily, the APOBEC2 subfamily, the APOBEC3 subfamily (e.g., the 3A subfamily, the 3B subfamily, the 3C subfamily, the 3D subfamily, the 3F subfamily, the 3G subfamily, or the 3H subfamily), or the APOBEC4 subfamily. An ACD of the present disclosure can be based on a member of the APOBEC protein family from a vertebrate, such as a mammal. Examples of mammals include, but are not limited to, rodents, primates, rabbit, bovine (e.g., cow), porcine (e.g., pig), equine (e.g., horse), elephant, and aardvark. An example of a primate is a human and a chimpanzee.

The APOBEC protein family is a member of the large cytidine deaminase superfamily that contains a canonical zinc-dependent deaminase (ZDD) signature motif embedded within a core cytidine deaminase fold. This fold includes a five-stranded mixed beta (b)-sheet surrounded by six alpha (a)-helices with the order a1-b1-b2-a2-b3-a3-b4-a4-b5-a5-a6 (Salter et al., Trends Biochem Sci. 2016 41 (7): 578-594. Doi: 10.1016/j.tibs.2016.05.001; Salter et al., Trends Biochem. Sci. 2018, 43 (8): 606-622 doi.org/10.1016/j.tibs.2018.04.013). Each cytidine deaminase domain core structure of APOBEC proteins contains a highly conserved spatial arrangement of the catalytic center residues of a zinc-binding motif H-[P/A/V]-E-X_{[23-28]}-P-C-X_{[2-4]}-C (SEQ ID NO:12) (referred to herein as the ZDD motif, where X is any amino acid, and the subscript range of numbers after X refers to the number of amino acids) (Salter et al., Trends Biochem Sci. 2016 41 (7): 578-594. Doi: 10.1016/j.tibs.2016.05.001). Without intending to be limited by theory, the H and two C residues coordinate a Zn atom, and the E residue polarizes a water molecule near the Zn-atom for catalysis (Chen et al., 2021, Viruses, 13:497, doi.org/10.3390/v13030497).

Some members of the APOBEC protein family, e.g., the AID subfamily, the APOBEC1 subfamily, the APOBEC2 subfamily, the APOBEC3A subfamily, the APOBEC3C subfamily, the APOBEC3H subfamily, and the APOBEC4 subfamily, include one copy of the ZDD motif. Other members of the APOBEC protein family, e.g., the APOBEC3B subfamily, the APOBEC3D subfamily, the APOBEC3F subfamily, and the APOBEC3G subfamily, include two copies of the ZDD motif, but often only the C-terminal copy is active (Salter et al., Trends Biochem Sci. 2016 41 (7): 578-594. Doi: 10.1016/j.tibs.2016.05.001). Thus, an altered cytidine deaminase disclosed herein includes one or two ZDD motifs. In one embodiment, an altered cytidine deaminase based on a member of the APOBEC3A subfamily that includes the following ZDD motif: HXEX₂₄SW(S/T)PCX_{[2-4]}CX₆FX₈LX₅R(L/I)YX_{[8-11]}LX₂LX_{[10]}M (SEQ ID NO:13) (where X is any amino acid, and the subscript number or range of numbers after X refers to the number of amino acids) (Salter et al., Trends Biochem Sci. 2016 41 (7): 578-594. Doi: 10.1016/j.tibs.2016.05.001).

In one embodiment, an ACD disclosed herein is a member of the APOBEC3 subfamily, e.g., APOBEC3A, APOBEC3B, APOBEC3C, APOBEC3D, APOBEC3F, or APOBEC3G, and can include one or more highly conserved sites that are part of the active site and within the ZDD motif SEQ ID NO:12. The sites include tryptophan at position 98 and serine or threonine at position 99 of SEQ ID NO:3 (Kouno et al., 2017, Nat. Comm, 8:15024, DOI: 10.1038/ncomms15024).

In addition to the ZDD motif, a member of the APOBEC protein family also includes other highly conserved residues that are part of the active site but not present as part of the ZDD motif SEQ ID NO: 12. A member the APOBEC3A subfamily, APOBEC3B subfamily, APOBEC3C subfamily, APOBEC3D subfamily, APOBEC3F subfamily, and APOBEC3G subfamily typically includes one or more of the following highly conserved sites that are part of the active site: arginine at position 28; histidine, asparagine, or arginine at position 29; serine or threonine, preferably threonine, at position 31; asparagine or aspartic acid at position 57; histidine at position 70; cysteine at position 101; cysteine at position 106; tyrosine or phenylalanine at position 130; asparagine or tyrosine at position 131; asparagine, tyrosine, or phenylalanine, preferably tyrosine, at position 132; and arginine or lysine at position 189 of SEQ ID NO: 3 (Kouno et al., 2017, Nat. Comm, 8:15024, DOI: 10.1038/ncomms15024).

An ACD of the present disclosure includes a substitution mutation, deletion or insertion at one or more residues when compared to a reference cytidine deaminase. A substitution mutation can be at the same position or a functionally equivalent position compared to the reference cytidine deaminase. Reference cytidine deaminases and functionally equivalent positions are described in detail herein. As noted, positions of the altered amino acids described herein and in the tables are in reference to APOBECA3A (SEQ ID NO:3) but one skilled in the art is capable of deriving the functionally equivalent positions in the other referenced cytosine deaminases. For example, in one embodiment, altered cytidine deaminases may contain the C-selectivity enhancing mutations in the backbones other than APOBECA3A (e.g., any one of those described herein, for example, those listed in Table 1). In another example, the altered cytosine deaminase may contain a combination of one or more mutations that increase the stability of the enzyme. Those one or more stability mutations described herein can be inserted into the other backbone cytoside deaminases described herein. The skilled person will readily appreciate that an altered cytidine deaminase described herein is not naturally occurring.

A reference cytidine deaminase can be a member of the APOBEC protein family. Essentially any known member of the APOBEC protein family can be a reference cytidine deaminase. The skilled person can easily identify members of each of the subfamilies by using a publicly available database such as the Protein database available at the National Center for Biotechnology Information (ncbi.nlm.nih.gov/protein) and searching for APOBEC1, APOBEC2, APOBEC3A, APOBEC3B, APOBEC3C, APOBEC3D, APOBEC3F, APOBEC3G, APOBEC3H, APOBEC4, or, when identifying members of the AID family, Activation-induced cytidine deaminase. A wild-type reference cytidine deaminase has the activity of binding single-stranded DNA (ssDNA) and deaminating a cytosine present on the ssDNA to convert it to uracil. In one embodiment, a wild-type reference cytidine deaminase has the activity of binding single-stranded RNA (ssRNA) and deaminating a cytosine present on the ssRNA to convert it to uracil. Methods for determining whether a protein binds ssDNA or ssRNA and deaminates a cytosine present are known to the skilled person.

In one embodiment, an ACD has an amino acid sequence that is based on a reference sequence which is a member of the APOBEC protein family includes a ZDD motif H-[P/A/V]-E-X_{[23-28]}-P-C-X_{[2-4]}-C (SEQ ID NO:12) and at least one substitution mutation disclosed herein. Optionally, an altered cytidine deaminase includes other active site residues disclosed herein. Non-limiting examples of reference cytidine deaminase proteins are shown in the following table.

TABLE 1
Examples of members of the APOBEC protein subfamilies.

| APOBEC protein | |
|---|---|
| family | Non-limiting examples |
| AID | UniProt: Q9GZX7 (SEQ ID NO: 1); UniProt: G3QLD2 (SEQ ID |
| | NO: 37); Uniprot: Q9WVE0 (SEQ ID NO: 38) |
| APOBEC1 | UniProt: P41238 (SEQ ID NO: 2); NCBI XP_030856728.1 (SEQ |
| | ID NO: 39); Uniprot P51908 (SEQ ID NO: 40) |
| APOBEC2 | UniProt: Q9Y235 (SEQ ID NO: 4); Uniprot G3SGN8 (SEQ ID |
| | NO: 41); Uniprot Q9WV35 (SEQ ID NO: 42) |
| APOBEC3A | UniProt: P31941(SEQ ID NO: 3); GenBank: XP_045219544.1 |
| | (SEQ ID NO: 19) |
| | GenBank: AER45717.1 (SEQ ID NO: 20); GenBank: |
| | XP_003264816.1 (SEQ ID NO: 21); GenBank: PNI48846.1 (SEQ |
| | ID NO: 22); GenBank: ADO85886.1 (SEQ ID NO: 23) |
| APOBEC3B | UniProt: Q9UH17 (SEQ ID NO: 5); Uniprot G3QV16 (SEQ ID |
| | NO: 43); Uniprot F6M3K5 (SEQ ID NO: 44) |
| APOBEC3C | UniProt: Q9NRW3 (SEQ ID NO: 6); Uniprot Q694B5 (SEQ ID |
| | NO: 45); Uniprot B0LW74 (SEQ ID NO: 46) |
| APOBEC3D | UniProt: Q96AK3 (SEQ ID NO: 7); NCBI NP_001332895.1 (SEQ |
| | ID NO: 47); NCBI NP_001332931.1 (SEQ ID NO: 48) |
| APOBEC3F | UniProt: Q8IUX4 (SEQ ID NO: 8); Uniprot G3RD21 (SEQ ID |
| | NO: 49); Uniprot Q1G0Z6 (SEQ ID NO: 50) |
| APOBEC3G | UniProt: Q9HC16 (SEQ ID NO: 9); Uniprot Q694C1 (SEQ ID |
| | NO: 51); Uniprot U5NDB3 (SEQ ID NO: 52) |
| APOBEC3H | UniProt: Q6NTF7 (SEQ ID NO: 10); Uniprot B7T0U7 (SEQ ID |
| | NO: 53); Uniprot Q19Q52 (SEQ ID NO: 54) |
| APOBEC4 | UniProt: Q8WW27(SEQ ID NO: 11); NCBI XP_004028087.1 |
| | (SEQ ID NO: 55); Uniprot Q497M3 (SEQ ID NO: 56) |

UniProt, database of protein sequence and functional information, available at uniprot.org; GenBank, collection of nucleotide sequences and their protein translations, available at ncbi.nlm.nih.gov/protein/.

In one embodiment, an ACD has an amino acid sequence that is based on a reference sequence that is a member of the APOBEC3A subfamily, and includes a ZDD motif HXEX₂₄SW(S/T)PCX_{[2-4]}CX₆FX₈LX₅R(L/I)YX_{[8-11]}LX₂LX_{[10]}M (SEQ ID NO:13) (where X is any amino acid, and the subscript number or range of numbers after X refers to the number of amino acids) and at least one substitution mutation disclosed herein. In one embodiment, the substitution mutation is a substitution mutation at the underlined tyrosine, such as a substitution mutation to leucine (L) or phenylalanine (F). The underlined tyrosine (Y) of SEQ ID NO:13 is the position functionally equivalent to the tyrosine amino acid 130 of the APOBEC3A protein SEQ ID NO:3. Optionally, the altered cytidine deaminase includes other active site residues disclosed herein.

In one embodiment, the amino acid sequence of an ACD includes the amino acids of a member of the APOBEC3A subfamily: X_{[16-26]}-GRXXTXLCYXV-X₁₅-GXXXN-X₁₂-HAEXXF-X₁₄-YXXTWXXSWSPC-X_{[2-4]}-CA-X₅-FL-X₇-LXIXXXR(L/I)Y̅-X₈-GLXXLXXXG-X₅-M-X₄-FXXCWXXFV-X₆-FXPW-X₁₃-LXXI-X_{[2-6]} (SEQ ID NO:14) (where X is any amino acid, and the subscript number or range of numbers after X refers to the number of amino acids), or a subset thereof, and at least one substitution mutation disclosed herein. The underlined tyrosine (Y) of SEQ ID NO: 14 is the position functionally equivalent to the tyrosine amino acid 130 of the APOBEC3A protein SEQ ID NO:3. In one embodiment, the substitution mutation is a substitution mutation at the underlined tyrosine, such as a substitution mutation to leucine (L) or phenylalanine (F). Optionally, the altered cytidine deaminase includes other active site residues disclosed herein.

In one embodiment, the amino acid sequence of an ACD includes the amino acids of a member of the APOBEC3A subfamily: X₂₆-GRXXTXLCYXV-X₁₅-G-X₁₆-HAEXXF-X₁₄-YXXTWXXSWSPC-X₄-CA-X₅-FL-X₇-LXIFXXR(L/I)Y-X₈-GLXXLXXXG-X₅-M-X₄-FXXCWXXFV-X₆-FXPW-X₁₃-LXXI-X₆ (SEQ ID NO:15) (where X is any amino acid, and the subscript number after X refers to the number of amino acids present), or a subset thereof, and at least one substitution mutation disclosed herein. The underlined tyrosine (Y) of SEQ ID NO:15 is the position functionally equivalent to the tyrosine amino acid 130 of the APOBEC3A protein SEQ ID NO:3. In one embodiment, the substitution mutation is a substitution mutation at the underlined tyrosine (Y), such as a substitution mutation to leucine (L) or phenylalanine (F). Optionally, the altered cytidine deaminase includes other active site residues disclosed herein.

A substitution mutation can be at the same position or a functionally equivalent position compared to a reference cytidine deaminase. By “functionally equivalent” it is meant that the altered cytidine deaminase has the amino acid substitution at the amino acid position in a reference cytidine deaminase that has the same functional role in both the reference cytidine deaminase and the altered cytidine deaminase.

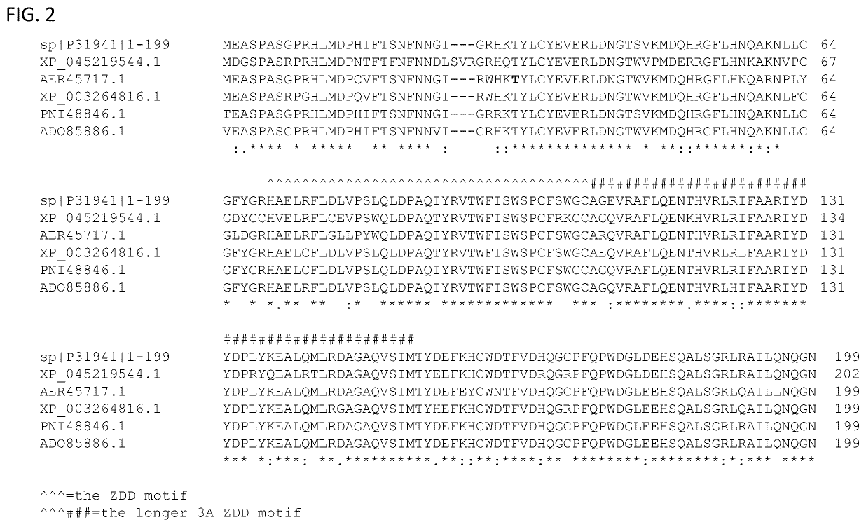

In general, functionally equivalent substitution mutations in two or more different cytidine deaminases occur at homologous amino acid positions in the amino acid sequences of the cytidine deaminases. Hence, use herein of the term “functionally equivalent” also encompasses mutations that are “positionally equivalent” or “homologous” to a given mutation, regardless of whether or not the particular function of the mutated amino acid is known. It is possible to identify the locations of functionally equivalent and positionally equivalent amino acid residues in the amino acid sequences of two or more different cytidine deaminases on the basis of sequence alignment and/or molecular modelling. An example of a sequence alignment to identify positionally equivalent and/or functionally equivalent residues is set forth in FIG. 2. For example, the residues in the members of the APOBEC3A subfamily in FIG. 2 that are vertically aligned are considered positionally equivalent as well as functionally equivalent to the corresponding residue in the human APOBEC3A amino acid sequence. Thus, for example, as shown in FIG. 2, the tyrosine at residue 130 of the APOBEC3A proteins of Homo sapiens, Pongo pygmaeus, Nomascus leucogenys, Pan troglodytes, and Gorilla gorilla and the tyrosine at residue 133 of the APOBEC3A protein from Macaca fascicularis are functionally equivalent and positionally equivalent. The skilled person can easily identify functionally equivalent residues in cytidine deaminases.

In one embodiment, an altered cytidine deaminase has an amino acid sequence that is structurally similar to a reference cytidine deaminase disclosed herein. In one embodiment, a reference cytidine deaminase is one that includes the amino acid sequence of a sequence listed in Table 1, SEQ ID NO:14, or SEQ ID NO:15. Other reference cytidine deaminases are described herein.

As used herein, an ACD may be “structurally similar” or have “structural similarity” to a reference cytidine deaminase if the amino acid sequence of the ACD possesses a specified amount of sequence similarity and/or sequence identity compared to the reference cytidine deaminase.

Structural similarity of two amino acid sequences can be determined by aligning the residues of the two sequences (for example, a candidate ACD and a reference cytidine deaminase described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate altered cytidine deaminase is the cytidine deaminase being compared to the reference cytidine deaminase. A candidate ACD that has structural similarity with a reference cytidine deaminase and cytidine deaminase activity is an altered cytidine deaminase.

Unless modified as otherwise described herein, a pair-wise comparison analysis of amino acid sequences can be conducted, for instance, by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2004). One example of an algorithm that is suitable for determining structural similarity is the BLAST® algorithm, which is described in Altschul et al., J. Mol. Biol. 215:403-410 (1990). The BLAST® algorithm can be used to calculate percent sequence identity and percent sequence similarity between two sequences. Software for performing BLAST® analyses is publicly available through the National Center for Biotechnology Information.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. Thus, in one embodiment the amino acid sequence of a cytidine deaminase protein having sequence similarity to a reference sequence may include conservative substitutions of amino acids present in that reference sequence.

A conservative substitution for an amino acid in a protein may be selected from other members of the class to which the amino acid belongs. For example, it is well-known in the art of protein biochemistry that an amino acid belonging to a grouping of amino acids having a particular size or characteristic (such as charge, hydrophobicity, or hydrophilicity) can be substituted for another amino acid without altering the activity of a protein, particularly in regions of the protein that are not directly associated with biological activity. For example, amino acids having a non-polar side chain include alanine, glycine, isoleucine, leucine, methionine, phenylalanine, proline, tryptophan, and valine; amino acids having a hydrophobic side chain include glycine, alanine, valine, leucine, isoleucine, proline, phenylalanine, methionine, and tryptophan; amino acids having a polar side chain include arginine, asparagine, aspartic acid, glutamine, glutamic acid, histidine, lysine, serine, cysteine, tyrosine, and threonine; and amino acids having an uncharged side chain include glycine, serine, cysteine, asparagine, glutamine, tyrosine, and threonine.

Thus, as used herein, reference to a cytidine deaminase as described herein, such as reference to the amino acid sequence of one or more SEQ ID NOs described herein can include a protein having structural similarity to the reference cytidine deaminase, e.g., at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference cytidine deaminase. Examples of ACDs having similarity with a reference amino acid sequence includes those having, for instance, (i) at least 60% to at least 99% similarity with SEQ ID NO:3, (ii) at least 60% to at least 99% similarity with SEQ ID NO: 78, (iii) at least 60% to at least 99% similarity with SEQ ID NO:80, (iv) at least 60% to at least 99% similarity with SEQ ID NO:81, and (v) at least 60% to at least 99% similarity with SEQ ID NO:82.

Alternatively, as used herein, reference to a cytidine deaminase as described herein, such as reference to the amino acid sequence of one or more SEQ ID NOs described herein can include a protein having structural similarity to the reference cytidine deaminase, e.g., at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference cytidine deaminase. Examples of ACDs having identity with a reference amino acid sequence includes those having, for instance, (i) at least 60% to at least 99% identity with SEQ ID NO: 3, (ii) at least 60% to at least 99% identity with SEQ ID NO:78, (iii) at least 60% to at least 99% identity with SEQ ID NO:80, (iv) at least 60% to at least 99% identity with SEQ ID NO:81, and (v) at least 60% to at least 99% identity with SEQ ID NO:82.

### Substitution Mutations Conferring Altered Cytidine Deaminase Activity

An altered cytidine deaminase of the present disclosure can include a substitution mutation at a position functionally equivalent to tyrosine at position 130 (Y130) in a member of the APOBEC protein family, including a member of the APOBEC3A subfamily (for instance, SEQ ID NO:3). In some APOBEC family proteins, the wild-type residue at a position functionally equivalent to Y130 is phenylalanine (F). Accordingly, in some ACDs the residue at a position functionally equivalent to Y130 can be described as (Tyr/Phe) 130 or Y/F130. As described herein, substitution mutations at this position can result in an ACD that preferentially acts on C compared to 5mC and 5hmC (i.e., has C-selective deaminase activity).

An altered cytidine deaminase of the present disclosure can include a substitution mutation at a position two, three, four, or five amino acids on the C-terminal side of the Y130 position, or functionally equivalent to the Y130 position. In one embodiment, the second mutation is at a position functionally equivalent to tyrosine at position 132 (Y132) in a member of the APOBEC protein family, including a member of the APOBEC3A subfamily (for instance, SEQ ID NO:3). In some embodiments, the substitution mutation at a position two, three, four, or five amino acids on the C-terminal side of the Y130 position, or functionally equivalent to the Y130 position is optionally present with a substitution mutation at a position functionally equivalent to tyrosine at position 130 (Y130). As described herein, a substitution mutation at this position alone or in combination with a substitution mutation at the Y130 position can further enhance the altered activity of an ACD.

In one embodiment, the substitution mutation at a position functionally equivalent to Y130 increases cytidine deaminase activity and preferentially acts on C compared to 5mC and 5hmC (i.e., has C-selective deaminase activity). The substitution mutation in an ACD is at a position functionally equivalent to position 130 in a member of the APOBEC protein family, including a member of the APOBEC3A subfamily (for instance, SEQ ID NO:3) and can be a mutation to any amino acid. In one embodiment, the substitution mutation in an ACD is at a position functionally equivalent to position 130 in a member of the APOBEC protein family is leucine (L), phenylalanine (F) or isoleucine (I).

Optionally, an ACD that preferentially acts on C compared to 5mC and 5hmC further includes a second substitution mutation at a position two, three, four, or five amino acids on the C-terminal side of the Y130 position, or functionally equivalent to the Y130 position. In one embodiment, the second mutation is at a position functionally equivalent to tyrosine at position 132 (Y132) in a member of the APOBEC protein family, including a member of the APOBEC3A subfamily (for instance, SEQ ID NO:3). The substitution mutation at the second position can be histidine (H) or proline (P). In one embodiment, the substitution mutation at the second position is histidine (H), such as Y132 to histidine (Y132H). Specific examples of ACDs that preferentially act on C compared to 5mC and 5hmC include those having the substitution mutations Y130L/Y132H and Y130F/Y132P. Another specific example of an ACD that preferentially acts on C compared to 5mC and 5hmC has the substitution mutation Y132P.

In some embodiments, the C-preferring ACD comprising a substitution at D133, wherein the substitution is any amino acid except P or Y, for example, K, D or R. These C-preferring may include, for example, Tyr130Leu/Tyr132His, Tyr130Phe/Tyr132Pro, Tyr132Pro, Tyr130Leu/Tyr132His/D133X, Tyr130Phe/Tyr132Pro/D133X, or Tyr132Pro/D133X, or Tyr130Ile/Tyr132Pro/D133X, wherein X is any amino acid except P, Y, preferably D, K, or R. These C-preferring ACD can further comprise one or more stabilizing mutations or alterations described herein, including two or more, three or more, four or more, five or more, six or more, seven or more, eight or more, nine or more, ten or more, eleven or more, twelve or more, thirteen or more, fourteen or more of the mutations selected from Table 4-6, for example, A112X, A126X, A139X, A148X, A185X, A192X, A59X, A87X, C106X, C161X, C171X, C34X, D145X, D156X, D163X, D167X, D177X, D180X, D41X, D77X, D85X, E109X, E116X, E138X, E157X, E38X, G105X, G108X, G188X, G25X, G27X, H119X, H11X, H16X, H182X, H29X, H51LX, I17X, I26X, I89X, K47X, K60X, L135X, L62X, L78X, M14X, M48X, N117X, N196X, N21X, N42X, P80X, Q115X, Q141X, Q169X, Q184X, R111X, R123X, R189X, R39X, R74X, R91X, S103X, S183X, S187X, S20X, S45X, T118X, T164X, T19X, T31X, T93X, V110X, V79X, L12X, D14X, T19X, G27X, R28X, E38X, F54X, H56X, N57X, Y67X, L73X, D77X, S81X, Y90X, I96X, S97X, C101X, F102X, W104X, C106X, A107X, L114X, V120X, L122X, R128X, Y136X, M142X, A146X, K159X, C161X, L186X, (Table 4). More examples of stability mutations are described below.

### Alterations Conferring Increased Selectivity or Stability

An ACD of the present disclosure can include at least one alteration, e.g., one or more substitution mutation, one or more deletion, and/or one or more insertion, that enhances selectivity or stability of an ACD. In some embodiments, the alteration may enhance both selectivity and stability of an ACD.

### Selectivity

An ACD having an alteration that enhances selectivity is one that preferentially deaminates C instead of 5mC and 5hmC. Thus, one or more alteration that enhances selectivity can be present in an ACD with one or more of the substitution mutations described herein, e.g., a substitution mutation at a position functionally equivalent to Y130, a substitution mutation at a position functionally equivalent to Y132, or substitution mutations at positions functionally equivalent to Y130 and to Y132. An ACD of the present disclosure that includes one or more alteration that enhances selectivity can also include one or more of the stability-enhancing alterations described herein.

An alteration that enhances selectivity of an ACD described herein for C can be (i) a substitution mutation at a position functionally equivalent to an amino acid, a deletion, or an insertion, (ii) a substitution mutation in combination with one or more deletions and one or more insertions, (iii) combinations of more than one selectivity-enhancing substitution mutation in combination with one or more deletion, one or more insertion, or in combination with both one or more deletion and one or more insertion. A selectivity-enhancing alteration is considered to enhance the selectivity of an ACD for C if it has greater deamination of C when compared to the same ACD that does not have the selectivity-enhancing alteration.

The locations of selectivity-enhancing alterations have been identified and the specific alterations predicted using various approaches. In one embodiment, a substitution mutation that is a selectivity-enhancing alteration is at a position functionally equivalent to the aspartic acid at position 133 (D133) in a member of the APOBEC protein family, including a member of the APOBEC3A subfamily (for instance, SEQ ID NO:3). The substitution mutation at D133 can be to any amino acid. In one example, D133 can be any amino acid except P or Y. In another example, D133 is selected from D, K and R. Other alterations that enhance the selectivity of an ACD for C include (i) a substitution mutation at a position functionally equivalent to S103X, E138X, Q115X, R28X, Y136X, C108X, or A112X, or a combination thereof, and (ii) a deletion of amino acids at positions functionally equivalent to I26-G27 (referred to as ΔI26-G27), where the position number designation is functionally equivalent to the position in a wild-type APOBEC3A (SEQ ID NO:3) and X is an amino acid substitution different from the wild-type amino acid at that position, or (iii) an insertion at one or more positions functionally equivalent to Σ130-131:X, Σ132-133:X, Σ130-131:X, Σ131-132:X, Σ131-132:X, Σ102-103:X, Σ138-139:X, Σ137-138:X, or Σ134-135:X, where the position number designation is functionally equivalent to the position in a wild-type APOBEC3A (SEQ ID NO:3) and X is the amino acid inserted between the two identified amino acids.

Other alterations that enhance the selectivity of an ACD for C, and increase the deamination of C instead of 5mC, are shown in Table 2 or 3. Thus, an ACD that preferentially deaminates C instead of 5mC and 5hmC can include a substitution mutation at a position functionally equivalent to D133, one or more alterations shown in Table 2, one or more alterations shown in Table 3, or a combination thereof, and further include a substitution mutation at a position functionally equivalent to Y130, Y132, or both Y130 and Y132. In one embodiment, the substitution mutation at a position functionally equivalent to Y130 can be a mutation to L or F. In another example, no substitution mutation at 130 can be present in the C-preferring ACD. In another example, the optional substitution mutation at a position functionally equivalent to Y132 can be a mutation to H or P. In another example, the substitution mutation at a position functionally equivalent to Y130 can be a mutation to F or I or no substitution mutation at 130 can be present, and the substitution mutation at a position functionally equivalent to Y132 can be a mutation to P. Specific examples of an ACD that has enhanced selectivity for C are those having the substitution mutations Y130L/Y132H/D133X, Y130F/Y132P/D133X, Y132P/D133X, Y130F/Y132P/D133X, and Y130I/Y132P/D133X, where X is any amino acid other than P or Y, and in some embodiments is W, D, K, or R. For example, an altered cytidine deaminase having enhanced selectivity for C can have at least 60% to at least 99% similarity or identity with SEQ ID NO:78, where the X at position 130 is Y, F, I, or L; the X at position 132 is P or H; and the X at 133 is W, D, K, or R. In some embodiments, an ACD having enhanced selectivity for C can have at least 60% to at least 99% similarity or identity with SEQ ID NO:78 where (i) the X at position 130 is L, the X at position 132 is H, and the X at 133 is W, D, K, or R, (ii) the X at position 130 is F, the X at position 132 is P, and the X at 133 is W, D, K, or R, (iii) the X at position 130 is Y, the X at position 132 is P, and the X at 133 is W, D, K, or R, or (iv) the X at position 130 is I, the X at position 132 is P, and the X at 133 is W, D, K, or R.

TABLE 2
Alterations that enhance selectivity for cytosine.

| Alteration tested¹ | Effect on C-selectivity² |
|---|---|
| S103K | ++ |
| S103Y | ++ |
| S103H | ++ |
| S103R | ++ |
| S103L | ++ |
| S103M | ++ |
| S103I | ++ |
| E138W | ++ |
| E138A | ++ |
| Q115W | ++ |
| ΔI26-G27 | ++ |
| ΔI26-G27, R28E | ++ |
| R28V | + |
| Y136W | + |
| E138I | + |
| C108F | + |
| C108S | + |
| A112D | + |
| C108M | + |
| C108E | + |
| S103C | + |
| S103Q | + |
| Y136D | + |
| Σ130-131: Y | ++ |
| Σ132-133: E | ++ |
| Σ130-131: A | ++ |
| Σ131-132: F | ++ |
| Σ131-132: A | ++ |
| Σ102-103: A | ++ |
| Σ138-139: A | ++ |
| Σ137-138: A | ++ |
| Σ134-135: A | ++ |

¹Alteration tested shows the position of the substitution mutation and the identity of the substitution mutation tested at the position, the position of the deletion, or the position of the insertion and the identity of the insertion tested at the position. Insertions are named with an “Σ”, followed by the two residues that flank the insertion and the amino acid inserted. For instance, Σ130-131: Y means a tyrosine (Y) amino acid was inserted between the amino acids at positions 130 and 131. Each substitution mutation was tested in an APOBECA3A protein (SEQ ID NO: 3) that included the additional substitution mutations Y130A/Y132H/D133W/R74L/T19Y/C171A/G108C/G188R/G25K/S45W/I17T/A59P/K60R/Δ61-68 (SEQ ID NO: 75, ScK).
²++, the alteration increased selectivity; +, the alteration increased selectivity to a lesser degree.

TABLE 3
Alterations that enhance selectivity for cytosine.

| Substitution alterations tested | Effect on C- |
|---|---|
| at the position¹ | selectivity² |
| Σ25-26: P_K25G | |
| Σ25-26: P_K25D | |
| Σ25-26: P_K25E | |
| Σ25-26: P_K25R | + |
| Σ25-26: P | |
| Σ25-26: P_K25D_I26L | ++ |
| Σ25-26: P_K25E_I26L | |
| Σ25-26: P_K25R_I26L | |
| Σ25-26: P_I26L | |
| Σ25-26: S_K25G | |
| Σ25-26: S_K25D | + |
| Σ25-26: S | |
| Σ25-26: I_K25G | |
| Σ25-26: I_K25E | |
| Σ25-26: I | |
| Σ25-26: S_K25G_I26L | |
| Σ25-26: S_K25D_I26L | |
| Σ25-26: S_I26L | + |
| Σ25-26: I_K25G_I26Y | + |
| Σ25-26: I_K25E_I26Y | + |
| Σ25-26: I_I26Y | |
| Σ25-26: V_K25G | |
| Σ25-26: V_K25D | + |
| Σ25-26: V | |
| Σ25-26: V_K25G_I26L | |
| Σ25-26: V_K25D_I26L | |
| Σ25-26: V_I26L | |
| Σ25-26: A | |
| Σ25-26: VEY_K25G | + |
| Σ25-26: VEY_K25S | |
| Σ25-26: VEY | |
| Σ25-26: AAA | |
| Σ130-131: Y | ++ |
| Σ130-131: Y_D131H | ++ |
| Σ130-131: Y_D131F | |
| Σ130-131: Y_D131H_H132W | |
| Σ130-131: Y_D131F_H132W | |
| Σ130-131: Y_D131H_H132W_W133D_L135H | ++ |
| Σ130-131: Y_D131F_H132W_W133D_L135H | |
| Σ130-131: Y_A130Y_D131H_H132W_W133D_L135H | |
| Σ130-131: Y_A130Y_D131F_H132W_W133D_L135H | |
| Σ130-131: A | ++ |
| Σ131-132: F | ++ |
| Σ131-132: F_D131Y_H132W_W133D | |
| Σ131-132: F_D131Y_H132W_W133D_L135D | ++ |
| Σ131-132: F_A130Y_D131Y_H132W_W133D_L135D | |
| Σ131-132: A | |
| Σ132-133: E | ++ |
| Σ132-133: E_W133E | ++ |
| Σ132-133: E_H132W_W133E | ++ |
| Σ132-133: E_D131M_H132W_W133E | ++ |
| Σ132-133: E_D131M_H132W_W133E_L135E | ++ |
| Σ132-133: E_A130F_D131M_H132W_W133E_L135E | |
| Σ132-133: A | |
| Σ134-135: K | |
| Σ134-135: K_L135A | |
| Σ134-135: K_P134D_L135A | ++ |
| Σ134-135: K_W133E_P134D_L135A | |
| Σ134-135: K_H132C_W133E_P134D_L135A | |
| Σ134-135: K_D131F_H132C_W133E_P134D_L135A | ++ |
| Σ134-135: | |
| K_A130Y_D131F_H132C_W133E_P134D_L135A | |
| Σ134-135: A | ++ |
| Σ132-133: W | |
| Σ132-133: W_W133E | |
| Σ26-27: A | |
| Σ27-28: A | |
| Σ28-29: A | |
| Σ29-30: A | |
| Σ30-31: A | |
| Σ31-32: A | |
| Σ32-33: A | |
| Σ95-96: A | |
| Σ96-97: A | |
| Σ97-98: A | |
| Σ98-99: A | |
| Σ99-100: A | |
| Σ100-101: A | |
| Σ101-102: A | |
| Σ102-103: A | |
| Σ103-104: A | |
| Σ104-105: A | |
| Σ127-128: A | |
| Σ128-129: A | |
| Σ133-134: A | ++ |
| Σ135-136: A | |
| Σ136-137: A | ++ |
| Σ137-138: A | ++ |
| Σ138-139: A | |
| Σ25-26: A | |
| Σ25-26: C | + |
| Σ25-26: D | |
| Σ25-26: E | |
| Σ25-26: F | |
| Σ25-26: G | |
| Σ25-26: H | |
| Σ25-26: I | |
| Σ25-26: K | + |
| Σ25-26: L | + |
| Σ25-26: M | |
| Σ25-26: N | |
| Σ25-26: P | |
| Σ25-26: Q | |
| Σ25-26: R | |
| Σ25-26: S | |
| Σ25-26: T | + |
| Σ25-26: V | |
| Σ25-26: W | + |
| Σ25-26: Y | |
| Σ28-29: A | |
| Σ28-29: C | |
| Σ28-29: D | |
| Σ28-29: E | + |
| Σ28-29: F | |
| Σ28-29: G | |
| Σ28-29: H | |
| Σ28-29: I | |
| Σ28-29: K | |
| Σ28-29: L | |
| Σ28-29: M | |
| Σ28-29: N | + |
| Σ28-29: P | |
| Σ28-29: Q | |
| Σ28-29: R | |
| Σ28-29: S | |
| Σ28-29: T | |
| Σ28-29: V | |
| Σ28-29: W | |
| Σ28-29: Y | + |
| Σ103-104: A | |
| Σ103-104: C | |
| Σ103-104: D | |
| Σ103-104: E | |
| Σ103-104: F | + |
| Σ103-104: G | |
| Σ103-104: H | |
| Σ103-104: I | |
| Σ103-104: K | |
| Σ103-104: L | |
| Σ103-104: M | |
| Σ103-104: N | |
| Σ103-104: P | |
| Σ103-104: Q | |
| Σ103-104: R | |
| Σ103-104: S | |
| Σ103-104: T | |
| Σ103-104: V | |
| Σ103-104: W | |
| Σ103-104: Y | |
| Σ104-105: A | |
| Σ104-105: C | + |
| Σ104-105: D | |
| Σ104-105: E | |
| Σ104-105: F | |
| Σ104-105: G | |
| Σ104-105: H | + |
| Σ104-105: I | |
| Σ104-105: K | + |
| Σ104-105: L | |
| Σ104-105: M | |
| Σ104-105: N | |
| Σ104-105: P | |
| Σ104-105: Q | |
| Σ104-105: R | |
| Σ104-105: S | |
| Σ104-105: T | |
| Σ104-105: V | |
| Σ104-105: W_W104G/G105W | |
| Σ104-105: Y | |

¹Position of the substitution alteration and the identity of the insertion and/or substitution mutation tested at the position. Insertions are named with an “Σ”, followed by the two residues that flank the insertion and the amino acid inserted. For instance, Σ25-26: P_K25G means a proline (P) amino acid was inserted between the amino acids at positions 25 and 26. The substitution alterations were tested in a scaffold of an APOBECA3A protein (SEQ ID NO: 3) that included the additional substitution mutations Y130A/Y132H/D133W/R74L/T19Y/C171A/G108C/G188R/G25K/S45W/I17T/A59P/K60R/Δ61-68 (SEQ ID NO: 75, ScK). In some constructs one or more substitution mutation present in ScK was modified by substitution mutation, For instance, ScK carries the G25K mutation, but in the mutant Σ25-26: P_K25G, G25K was reverted back to G.
²++, the alteration increased selectivity; +, the alteration increased selectivity to a lesser degree.

### Stability

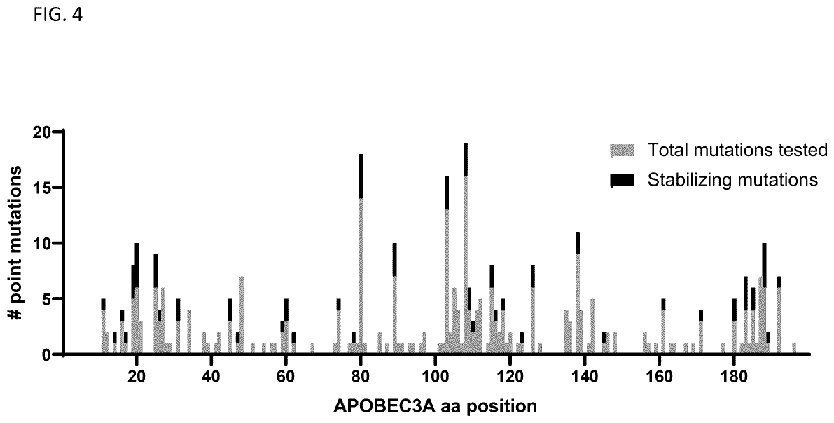

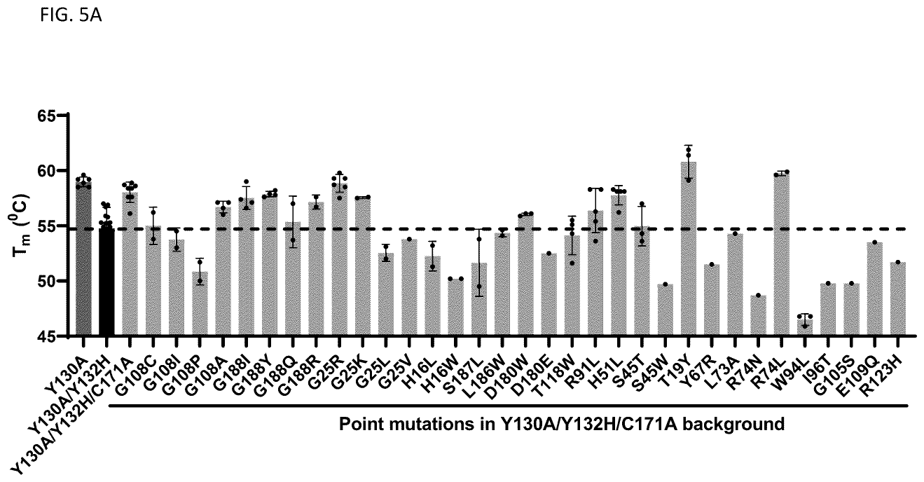

The present disclosure provides altered cytidine deaminases that have increased stability as compared with the wildtype enzyme protein. Experiments to identify stability-enhancing substitution alterations were initiated to address the reduced stability observed with some selectivity-enhancing alterations (e.g., a Y130/Y132/D133 triple mutation) (FIG. 4). In some embodiments, the stability-enhancing substitution alterations increase the thermal melting point of an ACD. Increased temperature optimums are highly desirable because it decreases DNA secondary structures by opening reaction sites that would be otherwise inaccessible due to secondary structure resulting in decreased false positive rate; stabilizes the enzyme in reaction conditions, which permits longer incubations and increased conversion; increases reaction kinetics, which allows for more tightly controlled conditions; and improved characteristics for commercialization, including increased shelf life, robustness in the assay, manufacturability, etc.

The stability-enhancing alterations described herein were identified in an ACD having 5mC-preferring activity, and are expected to stabilize ACDs that have C-preferring activity. The ability to stabilize the enzymatic protein allows for the increased stability of the enzyme in reaction conditions, thus allowing for increased conversion rates or activity, increased reaction kinetics and/or the ability to control conditions for reaction. Thus, in some embodiments, the ACD have one or more stability-enhancing alterations, two or more stability-enhancing alterations, three or more stability-enhancing alterations; four or more stability-enhancing alterations; five or more stability-enhancing alterations; six or more stability-enhancing alterations; seven or more stability-enhancing alterations; eight or more stability-enhancing alterations; nine or more stability-enhancing alterations; 10 or more stability-enhancing alterations; 11 or more stability-enhancing alterations; 12 or more stability-enhancing alterations; 13 or more stability-enhancing alterations; or 14 or more stability-enhancing alterations. Suitable stability-enhancing alterations can be found in Tables 4, 5, and 6, and can be combined in any number of combinations, including those described in Tables 7 and 8.

A substitution mutation that enhances stability can be present in an ACD with one or more of the alterations described herein, e.g., a substitution mutation at a position functionally equivalent to Y130, a substitution mutation at a position functionally equivalent to Y132, or substitution mutations at positions functionally equivalent to Y130 and to Y132 (e.g., SEQ ID NO: 78, SEQ ID NO:80, SEQ ID NO: 81, or SEQ ID NO:82). An alteration that enhances stability can be present in an ACD that includes one or more of the selectivity-enhancing substitution mutations described herein, including but not limited to a substitution mutation at a position functionally equivalent to D133, such as D133W/D/K/R, an alteration shown in Table 2, an alteration shown in Table 3, or a combination thereof.

A stability-enhancing alteration can be a substitution mutation at a position functionally equivalent to an amino acid, a deletion, or an insertion; a substitution mutation in combination with one or more deletions or one or more insertions; and combinations of one or more stability-enhancing substitution mutations in combination with one or more deletion, one or more insertion, or in combination with both one or more deletion and one or more insertion. A stability-enhancing alteration (e.g., substitution mutation, deletion, or insertion) is considered to enhance the stability of an ACD if it increases the melting temperature of the ACD. A substitution mutation, deletion, or insertion is considered to be stability-enhancing if it can increase the melting temperature of an ACD by at least 1° C., at least 2° C., at least 3° C., at least 4° C., at least 5° C., or at least 6° C. compared to the same ACD that does not have the stability-enhancing substitution mutation. A suitable method of determining the melting temperature of an ACD described herein is by fluorimetry (see Examples 1 and 4). However, it is well within one skilled in the art to determine the melting temperature of the ACDs described herein.

The locations of stability-enhancing substitution mutations have been identified and the specific substitutions predicted using various approaches including rational design, phylogenetic analysis, energy calculations, interface stabilization, EVmutation (Hopf et al., Nat Biotechnol. 2017 February; 35 (2): 128-135. doi: 10.1038/nbt.3769.), and EVcoupling (Hopf et al., Bioinformatics, 2019 May 1; 35 (9): 1582-1584. doi: 10.1093/bioinformatics/bty862.). Suitable examples of locations of stability-enhancing substitutions can be found in Table 4 and can be combined in any number of combinations to provide improved stability. In some examples, the locations of amino acids in an APOBEC3A (SEQ ID NO:3) where substitution mutation result in increased stability include A112X, A126X, A139X, A148X, A185X, A192X, A59X, A87X, C106X, C161X, C171X, C34X, D145X, D156X, D163X, D167X, D177X, D180X, D41X, D77X, D85X, E109X, E116X, E138X, E157X, E38X, G105X, G108X, G188X, G25X, G27X, H119X, H11X, H16X, H182X, H29X, H51LX, I17X, I26X, I89X, K47X, K60X, L135X, L62X, L78X, M14X, M48X, N117X, N196X, N21X, N42X, P80X, Q115X, Q141X, Q169X, Q184X, R111X, R123X, R189X, R39X, R74X, R91X, S103X, S183X, S187X, S20X, S45X, T118X, T164X, T19X, T31X, T93X, V110X, V79X, L12X, D14X, T19X, G27X, R28X, E38X, F54X, H56X, N57X, Y67X, L73X, D77X, S81X, Y90X, I96X, S97X, C101X, F102X, W104X, C106X, A107X, L114X, V120X, L122X, R128X, Y136X, M142X, A146X, K159X, C161X, L186X, (Table 4), and any combination thereof where the position number designation is functionally equivalent to the position in a wild-type APOBEC3A (SEQ ID NO:3) and X is an amino acid substitution different from the wild-type amino acid at that position. As discussed herein, one or more of these stabilizing mutations may be contemplated in combination. Specific examples of substitution mutations identified as stabilizing in different scaffolds are shown in Table 5.

In one embodiment, the disclosure provides a stabilized ACD, the stabilized ACD comprising one or more stability-enhancing alterations, two or more stability-enhancing alterations, three or more stability-enhancing alterations; four or more stability-enhancing alterations; five or more stability-enhancing alterations; six or more stability-enhancing alterations; seven or more stability-enhancing alterations; eight or more stability-enhancing alterations; nine or more stability-enhancing alterations; 10 or more stability-enhancing alterations; 11 or more stability-enhancing alterations; 12 or more stability-enhancing alterations; 13 or more stability-enhancing alterations; or 14 or more stability-enhancing alterations selected from Table 4, including, for example, those listed in Table 5, 6, 7 or 8. In one example, the stabilized ACD comprises one or more stability-enhancing alterations, two or more stability-enhancing alterations, three or more stability-enhancing alterations; four or more stability-enhancing alterations; five or more stability-enhancing alterations; six or more stability-enhancing alterations; seven or more stability-enhancing alterations; eight or more stability-enhancing alterations; nine or more stability-enhancing alterations; 10 or more stability-enhancing alterations; 11 or more stability-enhancing alterations; 12 or more stability-enhancing alterations; 13 or more stability-enhancing alterations; or 14 or more stability-enhancing alterations selected from A112X, A126X, A139X, A148X, A185X, A192X, A59X, A87X, C106X, C161X, C171X, C34X, D145X, D156X, D163X, D167X, D177X, D180X, D41X, D77X, D85X, E109X, E116X, E138X, E157X, E38X, G105X, G108X, G188X, G25X, G27X, H119X, H11X, H16X, H182X, H29X, H51LX, I17X, I26X, I89X, K47X, K60X, L135X, L62X, L78X, M14X, M48X, N117X, N196X, N21X, N42X, P80X, Q115X, Q141X, Q169X, Q184X, R111X, R123X, R189X, R39X, R74X, R91X, S103X, S183X, S187X, S20X, S45X, T118X, T164X, T19X, T31X, T93X, V110X, V79X, L12X, D14X, T19X, G27X, R28X, E38X, F54X, H56X, N57X, Y67X, L73X, D77X, S81X, Y90X, I96X, S97X, C101X, F102X, W104X, C106X, A107X, L114X, V120X, L122X, R128X, Y136X, M142X, A146X, K159X, C161X, and L186X. In another example, the stabilized ACD comprises one or more stability-enhancing alterations, two or more stability-enhancing alterations, three or more stability-enhancing alterations; four or more stability-enhancing alterations; five or more stability-enhancing alterations; six or more stability-enhancing alterations; seven or more stability-enhancing alterations; eight or more stability-enhancing alterations; nine or more stability-enhancing alterations; 10 or more stability-enhancing alterations; 11 or more stability-enhancing alterations; 12 or more stability-enhancing alterations; 13 or more stability-enhancing alterations; or 14 or more stability-enhancing alterations selected from A112H, A126C, A126F, A126I, A126L, A139G, A148V, A185F, A185H, A185I, A185R, A192E, A192I, A192K, A192L, A192R, A192W, A59P, A87R, C106I, C106S, C106T, C161I, C161L, C161M, C161V, C171A, C171I, C171R, C34I, C34L, D145R, D14Y, D156P, D156W, D163W, D177E, D180E, D180H, D180W, D41K, D77R, D85I, D85L, E109L, E109Q, E109W, E116L, E138A, E138L, E138R, E138W, E157W, E38W, E38Y, G105L, G108A, G108C, G108D, G108H, G108L, G108M, G108P, G108Q, G108R, G108W, G108Y, G188Y, G188A, G188I, G188L, G188Q, G188R, G25A, G25D, G25K, G25L, G25R, G25V, G27C, G27F, G27I, G27L, G27R, G27Y, H119W, H11L, H11P, H11R, H16D, H16L, H182N, H29R, H51L, I17T, I26A, I26L, I26R, I89C, I89D, I89H, I89Q, I89R, I89R, I89S, I89T, K47P, K47P, K60G, K60P, K60R, L135F, L62P, L78H, M142R, M142T, M48D, M48E, M48K, M48L, M48N, M48Q, M48S, N117W, N196P, N21H, N21Q, N21W, N42D, N42G, P80D, P80E, P80F, P80G, P80H, P80K, P80L, P80Q, P80R, ΔM1-L12, ΔN61-G68, ΔW104-G105, ΔQ195-N199, or ΔI26-G27, P80S, P80T, P80V, P80W, P80Y, Q115C, Q115K, Q115W, Q115Y, Q141R, Q169R, Q184V, R111A, R111V, R123H, R189K, R39L, R74C, R74L, R91L, S103F, S103H, S103H, S103R, S103T, S103W, S103Y, S103Y, S183F, S183V, S183W, S183Y, S187E, S187F, S187I, S187Q, S187W, S187Y, S20E, S20F, S20F, S20H, S20I, S20W, S20Y, S45R, S45T, S45W, T118P, T164I, T19I, T19L, T19W, T19Y, T31F, T31I, T31W, T93I, V110L, V110W, V79L, I17T, T19Y, G25K, A59P, K60R, R74L, G108C, C171A, G188R, and combinations thereof.

In one set of embodiments, the ACD comprises a combination of stabilizing mutations selected from I17X, T19X, G25X, S45X, A59X, K60X, Δ61-68, R74X, Δ104, Δ105, G108X, A126X, C171X, and G188X, wherein X is an amino acid different than the amino acid in a wildtype backbone. In one embodiment the ACD comprises I17X, T19X, G25X, S45X, A59X, K60X, Δ61-68, R74X, Δ104, Δ105, G108X, A126X, C171X, and G188X, wherein X is an amino acid different than the amino acid in a wildtype backbone.

For example, in one embodiment, the stabilized ACD comprises a combination of mutations I17T, T19Y, G25R/K, S45W, A59P, K60R, Δ61-68, R74L, Δ104, Δ105, G108A/C, A126C, C171A, G188R, and combinations thereof where the position number designation is functionally equivalent in a cytidine deaminase to the position in APOBEC3A (SEQ ID NO:3). For example, suitable combinations of the stabilizing mutations within the ACD can include, but are not limited to, for example, (a) I17T, T19Y, G25K, S45W, A59P, K60R, deletion of 61-68, R74L, deletion of G105, G108C, C171A, and G188R; (b) I17T, T19Y, G25K, S45W, A59P, K60R, deletion of 61-68, R74L, G108C, C171A, and G188R; (c) I17T, T19Y, G25K, S45W, A59P, K60R, deletion of 61-68, R74L, deletion of W104, deletion of G105, G108C, C171A, and G188R; (d) I17T, T19Y, G25K, S45W, A59P, K60R, deletion of 61-68, R74L, deletion of G105, G108C, (e) C171A, and G188R; I17T, T19Y, G25K, S45W, A59P, K60R, deletion of 61-68, R74L, G108C, C171A, and G188R; or (f) I17T, T19Y, G25K, S45W, A59P, K60R, deletion of 61-68, R74L, G108C, C171A, and G188R, where the positions are in the functionally equivalent positions within the cytidine deaminase as compared to SEQ ID NO:3 (APOBEC A3A backbone). However, this list is not exhaustive and other combinations of stability mutations are contemplated, both alone and in combination with selectivity mutations described herein.

TABLE 4
Stability-enhancing positions.
Substitution mutation position¹

H11X
L12X
D14X
H16X
I17X
T19X
S20X
N21X
G25X
I26X
G27X
R28X
H29X
T31X
C34X
E38X
R39X
D41X
N42X
S45X
K47X
M48X
H51X
F54X
H56X
N57X
A59X
K60X
L62X
Y67X
L73X
R74X
D77X
L78X
V79X
P80X
S81X
D85X
A87X
I89X
Y90X
R91X
T93X
W94X
I96X
S97X
C101X
F102X
S103X
W104X
G105X
C106X
A107X
G108X
E109X
V110X
R111X
A112X
L114X
Q115X
E116X
N117X
T118X
H119X
V120X
L122X
R123X
R128X
L135X
Y136X
E138X
A139X
Q141X
M142X
D145X
A146X
A148X
D156X
E157X
K159X
C161X
D163X
T164X
D167X
Q169X
C171X
D177X
D180X
H182X
S183X
Q184X
A185X
L186X
S187X
G188X
R189X
A192X
N196X
¹Position of the substitution mutation where the position number designation is functionally equivalent to the position in a wild-type APOBEC3A (SEQ ID NO: 3) and X is an amino acid substitution different from the wild-type amino acid at that position.

TABLE 5
Stability-enhancing substitution mutations.

| Substitution | | Mutation |
|---|---|---|
| mutation tested | | identified as |
| at the position¹ | Scaffold² | stabilizing³ |
| A112D | ScK | − |
| A112F | ScK | − |
| A112H | ScK | + |
| A112T | ScK | − |
| A112Y | ScK | − |
| A126C | ScK | +++ |
| A126F | ScE | +++ |
| A126I | ScE | + |
| A126L | ScE | + |
| A126S | ScK | − |
| A126V | ScE | − |
| A139G | ScK | +++ |
| A139Q | ScK | − |
| A139V | ScK | − |
| A139W | ScK | − |
| A146E | ScK | − |
| A146G | ScK | − |
| A148L | ScK | − |
| A148V | ScE | + |
| A185F | ScE | + |
| A185H | ScE | + |
| A185I | ScE | +++ |
| A185R | ScK | +++ |
| A192E | ScK | +++ |
| A192I | ScK | +++ |
| A192K | ScK | +++ |
| A192L | ScK | +++ |
| A192R | ScK | +++ |
| A192W | ScK | + |
| A59P | ScE | + |
| A87R | ScE | + |
| C101W | ScK | − |
| C106I | ScE | + |
| C106K | ScK | − |
| C106S | ScE | + |
| C106T | ScE | + |
| C108E | ScK | − |
| C108F | ScK | − |
| C108K | ScK | − |
| C108M | ScK | − |
| C108S | ScK | − |
| C161I | ScE | + |
| C161L | ScE | + |
| C161M | ScE | +++ |
| C161V | ScE | + |
| C171A | ScB | +++ |
| C171I | ScE | + |
| C171R | ScK | +++ |
| C34I | ScE | + |
| C34L | ScE | + |
| C34R | ScK | − |
| D145R | ScK | +++ |
| D14Y | ScE | +++ |
| D156P | ScK | +++ |
| D156W | ScK | +++ |
| D163W | ScK | +++ |
| D177E | ScK | + |
| D180E | ScB | + |
| D180H | ScK | +++ |
| D180W | ScB | +++ |
| D41K | ScE | + |
| D77R | ScE | + |
| D85I | ScK | +++ |
| D85L | ScK | +++ |
| E109L | ScK | +++ |
| E109Q | ScB | +++ |
| E109T | ScK | − |
| E109W | ScK | +++ |
| E116F | ScK | − |
| E116L | ScK | +++ |
| E116V | ScK | − |
| E138A | ScK | +++ |
| E138F | ScK | − |
| E138I | ScK | − |
| E138L | ScK | + |
| E138M | ScK | − |
| E138P | ScK | − |
| E138Q | ScK | − |
| E138R | ScK | +++ |
| E138W | ScK | + |
| E157W | ScE | + |
| E38W | ScK | +++ |
| E38Y | ScK | +++ |
| F102N | ScK | − |
| G105K | ScK | − |
| G105L | ScE | + |
| G105N | ScK | − |
| G105S | ScB | − |
| G105W | ScK | − |
| G105Y | ScK | − |
| G108A | ScB | +++ |
| G108C | ScB | +++ |
| G108D | ScE | + |
| G108H | ScE | + |
| G108I | ScB | − |
| G108L | ScE | + |
| G108M | ScE | + |
| G108P | ScB | + |
| G108Q | ScE | + |
| G108R | ScE | + |
| G108W | ScE | + |
| G108Y | ScE | +++ |
| G188Y | ScE | +++ |
| G188A | ScE | + |
| G188I | ScB | +++ |
| G188L | ScE | − |
| G188Q | ScB | +++ |
| G188R | ScB | +++ |
| G25A | ScE | + |
| G25D | ScE | + |
| G25K | ScB | +++ |
| G25L | ScB | + |
| G25R | ScB | +++ |
| G25V | ScB | +++ |
| G27C | ScK | + |
| G27F | ScK | +++ |
| G27I | ScE | + |
| G27L | ScE | + |
| G27R | ScK | +++ |
| G27Y | ScK | +++ |
| H119W | ScE | + |
| H11E | ScK | − |
| H11L | ScE | +++ |
| H11P | ScE | + |
| H11R | ScE | + |
| H16D | ScK | +++ |
| H16L | ScB | + |
| H182N | ScK | +++ |
| H29R | ScK | + |
| H51L | ScB | +++ |
| H56R | ScK | − |
| I17T | ScE | +++ |
| I26A | ScK | +++ |
| I26L | ScK | +++ |
| I26R | ScE | +++ |
| I89C | ScE | + |
| I89D | ScE | + |
| I89H | ScE | +++ |
| I89Q | ScE | + |
| I89R | ScE | +++ |
| I89R | ScK | +++ |
| I89S | ScE | + |
| I89T | ScE | +++ |
| I96T | ScB | − |
| K159R | ScK | − |
| K47P | ScE | +++ |
| K47P | ScK | + |
| K60G | ScK | +++ |
| K60P | ScE | + |
| K60R | ScK | +++ |
| L114G | ScK | − |
| L122D | ScK | − |
| L12W | ScK | − |
| L135F | ScK | + |
| L135Q | ScK | − |
| L135R | ScK | − |
| L135W | ScK | − |
| L186W | ScB | − |
| L62P | ScE | +++ |
| L73A | ScB | − |
| L78H | ScE | +++ |
| M142A | ScK | − |
| M142L | ScK | − |
| M142R | ScK | + |
| M142T | ScK | + |
| M142W | ScK | − |
| M48D | ScK | +++ |
| M48E | ScK | +++ |
| M48K | ScK | +++ |
| M48L | ScK | +++ |
| M48N | ScK | +++ |
| M48Q | ScK | +++ |
| M48S | ScK | +++ |
| N117W | ScE | + |
| N117Y | ScK | − |
| N196P | ScE | + |
| N21H | ScE | + |
| N21Q | ScK | + |
| N21W | ScE | + |
| N42D | ScK | +++ |
| N42G | ScK | +++ |
| N57D | ScE | − |
| P80D | ScE | + |
| P80E | ScE | + |
| P80F | ScE | + |
| P80G | ScE | +++ |
| P80H | ScE | + |
| P80K | ScE | + |
| P80L | ScE | + |
| P80Q | ScE | + |
| P80R | ScE | +++ |
| P80S | ScE | +++ |
| P80T | ScE | +++ |
| P80V | ScE | + |
| P80W | ScE | + |
| P80Y | ScE | + |
| Q115C | ScK | + |
| Q115K | ScK | +++ |
| Q115L | ScK | − |
| Q115M | ScK | − |
| Q115W | ScK | + |
| Q115Y | ScK | +++ |
| Q141R | ScE | + |
| Q169R | ScK | + |
| Q184V | ScK | +++ |
| R111A | ScK | +++ |
| R111E | ScK | − |
| R111Q | ScK | − |
| R111V | ScK | +++ |
| R123H | ScB | +++ |
| R128Y | ScK | − |
| R189K | ScK | +++ |
| R28V | ScK | − |
| R39L | ScE | + |
| R74C | ScE | + |
| R74L | ScA | +++ |
| R74N | ScB | − |
| R91L | ScB | + |
| S103C | ScK | − |
| S103D | ScK | − |
| S103F | ScE | + |
| S103H | ScE | + |
| S103H | ScK | + |
| S103I | ScK | − |
| S103K | ScK | − |
| S103L | ScK | − |
| S103M | ScK | − |
| S103Q | ScK | − |
| S103R | ScE | +++ |
| S103R | ScK | − |
| S103T | ScK | +++ |
| S103W | ScK | +++ |
| S103Y | ScE | +++ |
| S103Y | ScK | +++ |
| S183F | ScE | +++ |
| S183V | ScK | +++ |
| S183W | ScE | +++ |
| S183Y | ScK | + |
| S187E | ScE | + |
| S187F | ScK | + |
| S187I | ScE | + |
| S187Q | ScK | +++ |
| S187W | ScE | + |
| S187Y | ScK | + |
| S20E | ScK | +++ |
| S20F | ScE | +++ |
| S20F | ScK | +++ |
| S20H | ScK | +++ |
| S20I | ScK | +++ |
| S20W | ScK | +++ |
| S20Y | ScK | +++ |
| S45R | ScE | +++ |
| S45T | ScB | + |
| S45W | ScB | +++ |
| S81W | ScK | − |
| S97L | ScK | − |
| S97Y | ScK | − |
| T118K | ScK | − |
| T118P | ScE | +++ |
| T118W | ScB | − |
| T164I | ScE | + |
| T19F | ScE | − |
| T19I | ScE | +++ |
| T19L | ScE | +++ |
| T19W | ScE | + |
| T19Y | ScB | +++ |
| T31F | ScE | + |
| T31I | ScE | +++ |
| T31W | ScE | +++ |
| T93I | ScE | + |
| V110L | ScK | +++ |
| V110W | ScK | +++ |
| V120H | ScK | − |
| V120N | ScK | − |
| V79L | ScE | + |
| W104L | ScK | − |
| W104R | ScK | − |
| W94L | ScB | − |
| Y136D | ScK | − |
| Y136K | ScK | − |
| Y136W | ScK | − |
| Y90G | ScB | − |

¹Position of the substitution mutation and the identity of the substitution mutation tested at the position. For instance, H11L means the histidine at position 11 was replaced with a leucine.
²Each substitution mutation was tested in an APOBECA3A protein (SEQ ID NO: 3) that included the additional substitution mutations: ScB, Y130A/Y132H/C171A (SEQ ID NO: 68); ScE, Y130A/Y132H/D133W/R74L/C171A/T19Y (SEQ ID NO: 72); ScK, Y130A/Y132H/D133W/R74L/T19Y/C171A/G108C/G188R/G25K/S45W/I17T/A59P/K60R/Δ61-68 (SEQ ID NO: 75); ScA, Y130A/Y132H/D133W (SEQ ID NO: 69).
³+++, the substitution mutation increased stability substantially; +, the substitution mutation increased stability; −, the substitution mutation did not increase stability.
See Examples 1-4.

A stability-enhancing alteration can be a deletion. Tolerance to engineered deletions and insertions can be correlated with the rigidity of secondary structures. Beta sheets and helices tend to be the least tolerant to deletions and insertions, while loops and unstructured regions can be more tolerant. Loops in an APOBEC3A were identified and subjected to deletion mutagenesis, and the location of stability-enhancing substitution mutations that are deletions were identified. In some cases, the deletions have been tested. In one embodiment, the deletions are at positions functionally equivalent to positions shown in Table 6 that were produced and analyzed.

In an embodiment, there is provided an altered cytidine deaminase with C-preferring activity comprising the mutation Y130L, F or I, Y132 H or P, and D 133 W, D, K, or R, and at least one additional stabilizing mutation selected from at least one of A112H, A126C, A126F, A126I, A126L, A139G, A148V, A185F, A185H, A185I, A185R, A192E, A192I, A192K, A192L, A192R, A192W, A59P, A87R, C106I, C106S, C106T, C161I, C161L, C161M, C161V, C171A, C171I, C171R, C34I, C34L, D145R, D14Y, D156P, D156W, D163W, D177E, D180E, D180H, D180W, D41K, D77R, D85I, D85L, E109L, E109Q, E109W, E116L, E138A, E138L, E138R, E138W, E157W, E38W, E38Y, G105L, G108A, G108C, G108D, G108H, G108L, G108M, G108P, G108Q, G108R, G108W, G108Y, G188A, G188I, G188Q, G188R, G25A, G25D, G25K, G25L, G25R, G25V, G27C, G27F, G27I, G27L, G27R, G27Y, H119W, H11L, H11P, H11R, H16D, H16L, H182N, H29R, H51L, I17T, I26A, I26L, I26R, I89C, I89D, I89H, I89Q, I89R, I89R, I89S, I89T, K47P, K47P, K60G, K60P, K60R, L135F, L62P, L78H, M142R, M142T, M48D, M48E, M48K, M48L, M48N, M48Q, M48S, N117W, N196P, N21H, N21Q, N21W, N42D, N42G, P80D, P80E, P80F, P80G, P80H, P80K, P80L, P80Q, P80R, P80S, P80T, P80V, P80W, P80Y, Q115C, Q115K, Q115W, Q115Y, Q141R, Q169R, Q184V, R111A, R111V, R123H, R189K, R39L, R91L, S103F, S103H, S103H, S103R, S103T, S103W, S103Y, S103Y, S183F, S183V, S183W, S183Y, S187E, S187F, S187I, S187Q, S187W, S187Y, S20E, S20F, S20H, S20I, S20W, S20Y, S45R, S45T, S45W, T118P, T164I, T19I, T19L, T19W, T19Y, T31F, T31I, T31W, T93I, V110L, V110W, V79L, or any combination thereof wherein the position number designation is functionally equivalent to the position in a wildtype APOBEC3A (SEQ ID NO:3).

In an embodiment, there is provided an C-preferring altered cytidine deaminase comprising the mutations Y130 L, F or I, Y132 H or P and at least one additional stability mutation selected from at least one of A112H, A126C, A126F, A126I, A126L, A139G, A148V, A185F, A185H, A185I, A185R, A192E, A192I, A192K, A192L, A192R, A192W, A59P, A87R, C106I, C106S, C106T, C161I, C161L, C161M, C161V, C171A, C171I, C171R, C34I, C34L, D145R, D14Y, D156P, D156W, D163W, D177E, D180E, D180H, D180W, D41K, D77R, D85I, D85L, E109L, E109Q, E109W, E116L, E138A, E138L, E138R, E138W, E157W, E38W, E38Y, G105L, G108A, G108C, G108D, G108H, G108L, G108M, G108P, G108Q, G108R, G108W, G108Y, G188A, G188I, G188Q, G188R, G25A, G25D, G25K, G25L, G25R, G25V, G27C, G27F, G27I, G27L, G27R, G27Y, H119W, H11L, H11P, H11R, H16D, H16L, H182N, H29R, H51L, I17T, I26A, I26L, I26R, I89C, I89D, I89H, I89Q, I89R, I89R, I89S, I89T, K47P, K47P, K60G, K60P, K60R, L135F, L62P, L78H, M142R, M142T, M48D, M48E, M48K, M48L, M48N, M48Q, M48S, N117W, N196P, N21H, N21Q, N21W, N42D, N42G, P80D, P80E, P80F, P80G, P80H, P80K, P80L, P80Q, P80R, P80S, P80T, P80V, P80W, P80Y, Q115C, Q115K, Q115W, Q115Y, Q141R, Q169R, Q184V, R111A, R111V, R123H, R189K, R39L, R74C, R74L, R91L, S103F, S103H, S103H, S103R, S103T, S103W, S103Y, S103Y, S183F, S183V, S183W, S183Y, S187E, S187F, S187I, S187Q, S187W, S187Y, S20E, S20F, S20H, S20I, S20W, S20Y, S45R, S45T, S45W, T118P, T164I, T19I, T19L, T19W, T19Y, T31F, T31I, T31W, T93I, V110L, V110W, V79L, or any combination thereof wherein the position number designation is functionally equivalent to the position in a wildtype APOBEC3A (SEQ ID NO:3). In some embodiments, the ACD further comprises D133K or D133R.

In an embodiment, there is provided an altered cytidine deaminase comprising at least one mutation selected from at least one of A112H, A126C, A126F, A126I, A126L, A139G, A148V, A185F, A185H, A185I, A185R, A192E, A192I, A192K, A192L, A192R, A192W, A59P, A87R, C106I, C106S, C106T, C161I, C161L, C161M, C161V, C171A, C171I, C171R, C34I, C34L, D145R, D14Y, D156P, D156W, D163W, D177E, D180E, D180H, D180W, D41K, D77R, D85I, D85L, E109L, E109Q, E109W, E116L, E138A, E138L, E138R, E138W, E157W, E38W, E38Y, G105L, G108A, G108C, G108D, G108H, G108L, G108M, G108P, G108Q, G108R, G108W, G108Y, G188A, G188I, G188Q, G188R, G25A, G25D, G25K, G25L, G25R, G25V, G27C, G27F, G27I, G27L, G27R, G27Y, H119W, H11L, H11P, H11R, H16D, H16L, H182N, H29R, H51L, I17T, I26A, I26L, I26R, I89C, I89D, I89H, I89Q, I89R, I89R, I89S, I89T, K47P, K47P, K60G, K60P, K60R, L135F, L62P, L78H, M142R, M142T, M48D, M48E, M48K, M48L, M48N, M48Q, M48S, N117W, N196P, N21H, N21Q, N21W, N42D, N42G, P80D, P80E, P80F, P80G, P80H, P80K, P80L, P80Q, P80R, P80S, P80T, P80V, P80W, P80Y, Q115C, Q115K, Q115W, Q115Y, Q141R, Q169R, Q184V, R111A, R111V, R123H, R189K, R39L, R74C, R74L, R91L, S103F, S103H, S103H, S103R, S103T, S103W, S103Y, S103Y, S183F, S183V, S183W, S183Y, S187E, S187F, S187I, S187Q, S187W, S187Y, S20E, S20F, S20H, S20I, S20W, S20Y, S45R, S45T, S45W, T118P, T164I, T19I, T19L, T19W, T19Y, T31F, T31I, T31W, T93I, V110L, V110W, V79L, or any combination thereof wherein the position number designation is functionally equivalent to the position in a wildtype APOBEC3A (SEQ ID NO:3).

TABLE 6
Stability-enhancing deletions

| | Exemplary Scaffold² | Effect |
|---|---|---|
| Deletion¹ | used in construction | (−/+/++)³ |
| ΔM1-L12 (the first | ScK and ScI | + |
| 12 amino acids of | | |
| human APOBECA3A | | |
| (SEQ ID NO: 3) | | |
| ΔE36-G53 | | − |
| ΔN42 | ScK | − |
| ΔN61-G68 | ScF | ++ |
| ΔP86 | ScK | − |
| ΔW104-G105 | ScK | ++ |
| ΔL135-K137 | ScK | +/increases |
| | | preference for C |
| ΔP172 | ScK | − |
| ΔS187-N199 | ScK | + |
| ΔI26-G27 | ScK | ++/increases |
| | | preference for C |

¹Position of the deletion in an APOBEC3A (SEQ ID NO: 3).
²Deletions in an APOBECA3A protein that included the Y130A and Y132H substitution mutations.
³(−) does not increase stability; (+) neutral effect on stability; (++) increases stability, some positions also increased selectivity as noted.

Also note that the one or more ACD described herein may include one or more of the mutations in Table 4 (substitutions), Table 5 (substitutions), or Table 6 (deletions) as they may confer other desirable or beneficial effects (e.g., solubility, expression) for the cytidine deaminase. Therefore, an ACD of the present disclosure may contain one or more deletions (e.g., ΔS187-N199) to provide additional beneficial properties and may be used in connection with the selectivity/stability mutations of the present invention.

Some deletions can further include one or more ancillary substitution mutations. An ACD described herein can include the Δ61-68 deletion and the ancillary substitution mutation at (i) the position functionally equivalent to A59 where the mutation is to proline (A59P) or leucine (A59L), (ii) the position functionally equivalent to K60 where the mutation is to arginine (K60R), glutamic acid ((K60E), glutamine (K60Q), or glycine (K60G), or (iii) the position functionally equivalent to R69 (i.e., the position 69 prior to the deletion of 61-68) where the mutation is to tyrosine (R69Y), asparagine (R69N, histidine (R69H), aspartic acid (R69D), or leucine (R69L). Specific combinations of the Δ61-68 deletion and ancillary substitution mutations are Δ61-68/A59P/K60R, Δ61-68/A59L/R69Y, Δ61-68/A59L/K60E/R69N, 461-68/A59P/K60E/R69H, Δ61-68/A59P/K60Q/R69H, Δ61-68/A59L/R69N, Δ61-68/R69D, Δ61-68/A59L/K60R, and Δ61-68/A59P/K60G/R69L. An ACD described herein can include the Δ104-105 deletion and the ancillary substitution mutation at the position functionally equivalent to F102 where the mutation is to arginine (F102R), the substitution mutation at the position functionally equivalent to S103 where the mutation is to asparagine (S103N), or both substitution mutations F102R and S103N. In another embodiment, an ACD described herein can include both the Δ104-105 deletion and the Δ1-12 deletion. Additional embodiments include: an ACD having the Δ104-105 deletion, the Δ1-12 deletion, and the substitution mutation F102R; an ACD having the Δ104-105 deletion, the Δ1-12 deletion, and the substitution mutation S103N; and an ACD having the Δ104-105 deletion, the Δ1-12 deletion, and the substitution mutations F102R and S103N.

An ACD having C-preferring activity described herein can further include one or more stability-enhancing alterations selected from I17T, T19Y, G25K, S45W, A59P, K60R, deletion of 61-68, R74L, deletion of W104, deletion of G105, G108C, A126C, C171A, G188R, or a combination thereof, where the substitution mutation or a deletion is at a position functionally equivalent to an amino acid in a member of the APOBEC protein family, including a member of the APOBEC3A subfamily (for instance, SEQ ID NO:3). An ACD having C-preferring activity described herein can further include the stability-enhancing alterations I17T, T19Y, G25K, S45W, A59P, K60R, deletion of 61-68, R74L, deletion of W104, deletion of G105, G108C, C171A, and G188R (SEQ ID NO:77 where the substitution mutation or a deletion is at a position functionally equivalent to an amino acid in a member of the APOBEC protein family, including a member of the APOBEC3A subfamily (for instance, SEQ ID NO:3). An ACD having C-preferring activity described herein can further include the stability-enhancing alterations I17T, T19Y, G25K, S45W, A59P, K60R, deletion of 61-68, R74L, G108C, C171A, and G188R (SEQ ID NO:117) where the substitution mutation or a deletion is at a position functionally equivalent to an amino acid in a member of the APOBEC protein family, including a member of the APOBEC3A subfamily (for instance, SEQ ID NO:3). An ACD having C-preferring activity described herein can further include the stability-enhancing alterations I17T, T19Y, G25K, S45W, A59P, K60R, deletion of 61-68, R74L, deletion of G105, G108C, C171A, and G188R (SEQ ID NO:124) where the substitution mutation or a deletion is at a position functionally equivalent to an amino acid in a member of the APOBEC protein family, including a member of the APOBEC3A subfamily (for instance, SEQ ID NO:3).

An ACD having a stability-enhancing alteration can further include one or more selectivity-enhancing alterations, such as a substitution mutation at D133 as described herein, or one or more alterations shown on Table 2 and Table 3, in any combination.

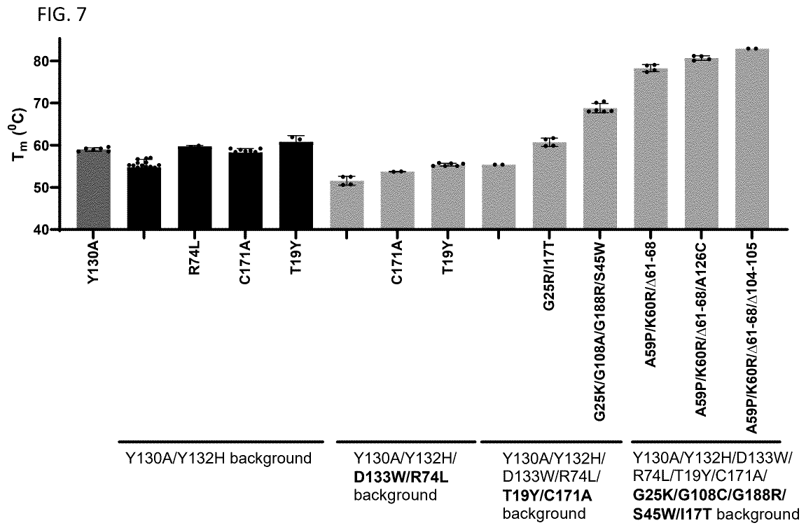

The inventors have observed that addition of stability-enhancing substitution alterations to an ACD that includes other stability-enhancing alterations results in an additive effect. Proteins can be dramatically stabilized without reducing biological function of the protein (Bloom et al., PNAS, 103 (15): 5869-5874), and this additive effect of stability-enhancing mutations has been observed by the inventors in ACD proteins (see FIG. 7). Accordingly, the inventors expect that addition of any subset of stability-enhancing alterations is possible and that they will result in increases in stability without sacrificing activity. Without intending to limit the particular combinations of stability-enhancing alterations encompassed by the present disclosure, specific examples of combinations of stability-enhancing alterations that can be present in a ACD having C-preferring activity (e.g., Y130L, Y130L/Y132H, Y132P, Y130F/Y132P, one or more alterations of Table 2, and/or one or more alterations of Table 3) include but are not limited to those shown in Tables 7 and 8. Many other combinations are contemplated and these Tables are not to be considered exhaustive. Some examples of the ACDs having C-preferring activity include, but are not limited to, for example, SEQ ID NO:78, SEQ ID NO:80, SEQ ID NO:81, SEQ ID NO:82, and at least one of the following combinations of stabilizing mutations, where the numbering of the nucleotides is based on SEQ ID NO:3: R74X/C171X; R74X/T19X; R74X/G25X; R74X/T19X/C171X; R74X/T19X/C171X; R74X/T19X/C171X/I17X; R74X/T19X/C171X/G25X; R74X/T19X/C171X/G25X/I17X; R74X/T19X/C171X/G25X/T19X; R74X/T19X/C171X/G25X; R74X/T19X/C171X/S45X; R74C/T19X/C171X; R74X/T19X/C171X/T19X; R74X/T19X/C171X/T19X; R74X/T19X/C171X/G108X; R74X/T19X/C171X/G108X; R74X/T19X/C171X/G108X; R74X/T19X/C171X/G108X; R74X/T19X/C171X/G108X; R74X/T19X/C171X/G108X; R74X/T19X/C171X/G108X; R74X/T19X/C171X/G108X; R74X/T19X/C171X/A126X; R74X/T19X/C171X; R74X/T19X/C171X/G108X; R74X/T19X/C171X/G108X; R74X/T19X/C171X/A126X; R74X/T19X/C171X/A126X; R74X/T19X/C171X/A126X; R74X/T19X/C171X; R74X/T19X/C171X/S45X; R74X/T19X/C171X/G25X; R74X/T19X/C171X/G25X; R74X/T19X/C171X/G188X; R74X/T19X/C171X/G188X; R74X/T19X/C171X/G188X; R74X/T19X/C171X/G108X; R74X/T19X/C171X/G108X/G188X/G25X/S45X; R74X/T19X/C171X/G108X; R74X/T19X/C171X/G108X/G188X/G25X/S45X/I17X; R74X/T19X/C171X/G108X/G188X/G25X/S45X/I17X/A59X/K60X/Δ61-68; R74X/T19X/C171X/G108X/G188X/G25X/S45X/I17X; R74X/T19X/C171X/G108X/G188X/G25X/S45X/I17X/A59X/K60X/Δ61-68/A126X; R74X/T19X/C171X/G108X/G188X/G25X/S45X/I17X/A59X/K60X/Δ61-68; I17X/T19X/G25X/S45X/A59X/K60X/deletion of 61-68/R74X/G108X/C171X/G188X; or I17X/T19X/G25KXS45X/A59X/K60X/deletion of 61-68/R74X/deletion of G105/G108X/C171X/G188X, wherein X is an amino acid substitution different than wildtype.

TABLE 7
Examples of ACDs with more than one
stability-enhancing alteration.

| Substitution mutations | Mutation identified as |
|---|---|
| tested at the position¹ | increasing stabilization² |
| G108A, | + |
| G108A, G188A, | + |
| G108A, G188A, G25K, | + |
| G108A, G188A, G25K, S45W | + |
| G108A, G188A, G25R, | + |
| G108A, G188A, G25R, S45W | + |
| G108A, G188A, S45W | +++ |
| G108A, G188Q, | + |
| G108A, G188Q, G25K, | + |
| G108A, G188Q, G25K, S45W | +++ |
| G108A, G188Q, G25R, | − |
| G108A, G188Q, G25R, S45W | +++ |
| G108A, G188Q, S45W | +++ |
| G108A, G188R, | + |
| G108A, G188R, G25K, | + |
| G108A, G188R, G25K, S45W | +++ |
| G108A, G188R, G25R, | + |
| G108A, G188R, G25R, S45W | +++ |
| G108A, G188R, S45W | +++ |
| G108A, G25K, | + |
| G108A, G25K, S45W | +++ |
| G108A, G25R, | + |
| G108A, G25R, S45W | + |
| G108A, S45W | + |
| G108C, | + |
| G108C, G188A, | + |
| G108C, G188A, G25K, | + |
| G108C, G188A, G25K, S45W | +++ |
| G108C, G188A, G25R, | + |
| G108C, G188A, G25R, S45W | +++ |
| G108C, G188A, S45W | +++ |
| G108C, G188Q, | + |
| G108C, G188Q, G25K, | + |
| G108C, G188Q, G25K, S45W | + |
| G108C, G188Q, G25R, | + |
| G108C, G188Q, G25R, S45W | + |
| G108C, G188Q, S45W | +++ |
| G108C, G188R, | + |
| G108C, G188R, G25K, | + |
| G108C, G188R, G25K, S45W | +++ |
| G108C, G188R, G25R, | + |
| G108C, G188R, G25R, S45W | +++ |
| G108C, G188R, S45W | +++ |
| G108C, G25K, | + |
| G108C, G25K, S45W | +++ |
| G108C, G25R, | + |
| G108C, G25R, S45W | +++ |
| G108C, S45W | + |
| G188A, | + |
| G188A, G25K, | + |
| G188A, G25K, S45W | + |
| G188A, G25R, | + |
| G188A, G25R, S45W | +++ |
| G188A, S45W | +++ |
| G188Q, | + |
| G188Q, G25K, | + |
| G188Q, G25K, S45W | +++ |
| G188Q, G25R, | + |
| G188Q, G25R, S45W | +++ |
| G188Q, S45W | + |
| G188R, | + |
| G188R, G25K, | + |
| G188R, G25K, S45W | +++ |
| G188R, G25R, | + |
| G188R, G25R, S45W | +++ |
| G188R, S45W | + |
| G25K, | + |
| G25K, S45W | +++ |
| G25R, | + |
| G25R, S45W | + |
| S45W | +++ |

Each substitution mutation was tested in an APOBECA3A protein (SEQ ID NO: 3) that included the additional substitution mutations Y130A/Y132H/D133W/R74L/C171A/T19Y (ScE, SEQ ID NO: 72).
¹Position of the substitution mutation and the identity of the substitution mutation tested at the position. For instance, S45W means the serine at position 45 was replaced with a tryptophan.
²+++, the substitution mutation increased stability substantially; +, the substitution mutation increased stability; −, the substitution mutation did not increase stability.
See Example 2.

In an embodiment, there is provided an altered cytidine deaminase comprising the mutation Y130 is L, F or I, Y132 is H or P, and Y133 is W, K, D or R and one or more of the following mutation combinations selected from, G108A; and/or G108A and G188A; and/or G108A, G188A and G25K; and/or G108A, G188A, G25K and S45W; and/or G108A, G188A and G25R; and/or G108A, G188A, G25R and S45W; and/or G108A, G188A and S45W; and/or G108A and G188Q; and/or G108A, G188Q and G25K; and/or G108A, G188Q, G25K and S45W; and/or G108A, G188Q, G25R and S45W; and/or G108A, G188Q and S45W; and/or G108A and G188R; and/or G108A, G188R and G25K; and/or G108A, G188R, G25K and S45W; and/or G108A, G188R and G25R; and/or G108A, G188R, G25R and S45W; and/or G108A, G188R and S45W; and/or G108A and G25K; and/or G108A, G25K and S45W; and/or G108A and G25R; and/or G108A, G25R and S45W; and/or G108A and S45W; and/or G108C; and/or G108C and G188A; and/or G108C, G188A and G25K; and/or G108C, G188A, G25K and S45W; and/or G108C, G188A and G25R; and/or G108C, G188A, G25R and S45W; and/or G108C, G188A and S45W; and/or G108C and G188Q; and/or G108C, G188Q and G25K; and/or G108C, G188Q, G25K and S45W; and/or G108C, G188Q and G25R; and/or G108C, G188Q, G25R and S45W; and/or G108C, G188Q and S45W; and/or G108C and G188R; and/or G108C, G188R and G25K; and/or G108C, G188R, G25K and S45W; and/or G108C, G188R and G25R; and/or G108C, G188R, G25R and S45W; and/or G108C, G188R and S45W; and/or G108C and G25K; and/or G108C, G25K and S45W; and/or G108C and G25R; and/or G108C, G25R and S45W; and/or G108C and S45W; and/or G188A; and/or G188A and G25K; and/or G188A, G25K and S45W; and/or G188A and G25R; and/or G188A, G25R and S45W; and/or G188A and S45W; and/or G188Q; G188Q and G25K; and/or G188Q, G25K and S45W; and/or G188Q and G25R; and/or G188Q, G25R and S45W; and/or G188Q and S45W; and/or G188R; G188R and G25K; and/or G188R, G25K and S45W; and/or G188R and G25R; and/or G188R, G25R and S45W; and/or G188R and S45W; and/or G25K; and/or G25K and S45W; and/or G25R; and/or G25R and S45W wherein the position number designation is functionally equivalent to the position in a wildtype APOBEC3A (SEQ ID NO:3).

In an embodiment, there is provided an altered cytidine deaminase comprising the mutation Y103L, Y130F or Y130I and Y132H or Y132P and one or more of the following stability mutation combinations selected from, G108A; and/or G108A and G188A; and/or G108A, G188A and G25K; and/or G108A, G188A, G25K and S45W; and/or G108A, G188A and G25R; and/or G108A, G188A, G25R and S45W; and/or G108A, G188A and S45W; and/or G108A and G188Q; and/or G108A, G188Q and G25K; and/or G108A, G188Q, G25K and S45W; and/or G108A, G188Q, G25R and S45W; and/or G108A, G188Q and S45W; and/or G108A and G188R; and/or G108A, G188R and G25K; and/or G108A, G188R, G25K and S45W; and/or G108A, G188R and G25R; and/or G108A, G188R, G25R and S45W; and/or G108A, G188R and S45W; and/or G108A and G25K; and/or G108A, G25K and S45W; and/or G108A and G25R; and/or G108A, G25R and S45W; and/or G108A and S45W; and/or G108C; and/or G108C and G188A; and/or G108C, G188A and G25K; and/or G108C, G188A, G25K and S45W; and/or G108C, G188A and G25R; and/or G108C, G188A, G25R and S45W; and/or G108C, G188A and S45W; and/or G108C and G188Q; and/or G108C, G188Q and G25K; and/or G108C, G188Q, G25K and S45W; and/or G108C, G188Q and G25R; and/or G108C, G188Q, G25R and S45W; and/or G108C, G188Q and S45W; and/or G108C and G188R; and/or G108C, G188R and G25K; and/or G108C, G188R, G25K and S45W; and/or G108C, G188R and G25R; and/or G108C, G188R, G25R and S45W; and/or G108C, G188R and S45W; and/or G108C and G25K; and/or G108C, G25K and S45W; and/or G108C and G25R; and/or G108C, G25R and S45W; and/or G108C and S45W; and/or G188A; and/or G188A and G25K; and/or G188A, G25K and S45W; and/or G188A and G25R; and/or G188A, G25R and S45W; and/or G188A and S45W; and/or G188Q; G188Q and G25K; and/or G188Q, G25K and S45W; and/or G188Q and G25R; and/or G188Q, G25R and S45W; and/or G188Q and S45W; and/or G188R; G188R and G25K; and/or G188R, G25K and S45W; and/or G188R and G25R; and/or G188R, G25R and S45W; and/or G188R and S45W; and/or G25K; and/or G25K and S45W; and/or G25R; and/or G25R and S45W wherein the position number designation is functionally equivalent to the position in a wildtype APOBEC3A (SEQ ID NO:3). In some embodiments, the ACD further comprise unmethylated profile. This enables the reaction to be run at higher temperatures where 5mC to T conversion is high and C to U conversion is low, providing a selectivity advantage under these conditions. In each case, the stability of a given construct and formulation can be quantified by Tmaxs D133K or D133R.

TABLE 8
Examples of ACDs with more than one stability-enhancing alteration.
Substitution mutations and deletions¹

A59P, K60R, Δ61-68
A59P, K60R, Δ61-68, Δ104-105
A59P, K60R, Δ61-68, Δ104-105, A126C
A59P, Δ61-68, Δ104-105, K60G
A59P, Δ61-68, Δ104-105, K60G, A126C
A59P, Δ61-68, Δ104-105, K60G, S103T
A59P, Δ61-68, Δ104-105, N21Q, K60G, A126C
A59P, Δ61-68, Δ104-105, K60G, S103T, A126C
A59P, Δ61-68, Δ104-105, K60G, R28V, S103T, A126C
A59P, Δ61-68, Δ104-105, N21Q, K60G, S103T, A126C
A59P, Δ61-68, Δ104-105, N21Q, R28V, K60G, S103T, A126C
Each substitution mutation is tested in an APOBECA3A protein (SEQ ID NO: 3) that optionally includes additional stability-enhancing alterations, such as the stability-enhancing alterations in SEQ ID NO: 68 (ScB), SEQ ID NO: 69 (ScA), SEQ ID NO: 70 (ScD), SEQ ID NO: 71 (ScC), SEQ ID NO: 72 (ScE), SEQ ID NO: 73 (ScL), SEQ ID NO: 74 (ScF), SEQ ID NO: 75 (ScK), SEQ ID NO: 76 (ScJ), or SEQ ID NO: 77 (ScI).
¹Position of the substitution mutation and the identity of the substitution mutation, position of deletions. For instance, A59P means the alanine at position 59 was replaced with a proline.

In some examples, the altered cytosine deaminases include, but are not limited to, for example, those having C-preferring deaminase activity (e.g., Y130L, Y130L/Y132H/D133W, Y132P/D133W, Y130F/Y132P/D133W, Y132P/D133W, one or more alterations of Table 2, and/or one or more alterations of Table 3), and at least one of the following combinations of stabilizing mutations, where the numbering is based on SEQ ID NO:3: R74L/C171A; R74L/T19Y; R74L/G25R; R74L/T19I/C171A; R74L/T19L/C171A; R74L/T19Y/C171A/I17T; R74L/T19Y/C171A/G25A; R74L/T19Y/C171A/G25R/I17T; R74L/T19Y/C171A/G25R/T19F; R74L/T19Y/C171A/G25D; R74L/T19Y/C171A/S45R; R74C/T19Y/C171A; R74L/T19Y/C171A/T19F; R74L/T19Y/C171A/T19W; R74L/T19Y/C171A/G108E; R74L/T19Y/C171A/G108D; R74L/T19Y/C171A/G108Q; R74L/T19Y/C171A/G108Y; R74L/T19Y/C171A/G108H; R74L/T19Y/C171A/G108L; R74L/T19Y/C171A/G108K; R74L/T19Y/C171A/G108R; R74L/T19Y/C171A/A126V; R74L/T19Y/C171I; R74L/T19Y/C171A/G108M; R74L/T19Y/C171A/G108W; R74L/T19Y/C171A/A126F; R74L/T19Y/C171A/A126I; R74L/T19Y/C171A/A126L; R74L/T19Y/C171A; R74L/T19Y/C171A/S45W; R74L/T19Y/C171A/G25R; R74L/T19Y/C171A/G25K; R74L/T19Y/C171A/G188Q; R74L/T19Y/C171A/G188A; R74L/T19Y/C171A/G188R; R74L/T19Y/C171A/G108A; R74L/T19Y/C171A/G108A/G188R/G25K/S45W; R74L/T19Y/C171A/G108C; R74L/T19Y/C171A/G108A/G188R/G25K/S45W/I17T; R74L/T19Y/C171A/G108A/G188R/G25K/S45W/I17T/A59P/K60R/Δ61-68; R74L/T19Y/C171A/G108C/G188R/G25K/S45W/I17T; R74L/T19Y/C171A/G108C/G188R/G25K/S45W/I17T/A59P/K60R/Δ61-68/A126C; R74L/T19Y/C171A/G108C/G188R/G25K/S45W/I17T/A59P/K60R/Δ61-68; I17T/T19Y/G25K/S45W/A59P/K60R/deletion of 61-68/R74L/G108C/C171A; or I17T/T19Y/G25K/S45W/A59P/K60R/deletion of 61-68/R74L/deletion of G105/G108C/C171A/G188R.

In one embodiment, examples of ACDs having one or more stability-enhancing alteration and optionally one or more selectivity-enhancing substitution mutation are shown at SEQ ID NO:196, where the position number designation is functionally equivalent to the position in a wild-type APOBEC3A (SEQ ID NO:3) and “X” is an amino acid having the same or a different identity from the wild-type amino acid at that position, or a deletion as noted. Where “X” is not defined, it may be any amino acid. As is shown in SEQ ID NO: 196, numerous amino acids may be mutated to enhance the stability of the ACD. While some mutations may be additive, some may be sufficient independently, depending on the desired activity of the ACD. Thus, the mutations indicated in SEQ ID NO: 196 are inclusive of many stability-enhancing mutations that may be made individually or in combination. SEQ ID NO: 198 is the sequence of one example of an ACD that preferentially deaminates unmodified cytosine shown with the numerous optional stability mutations of SEQ ID NO: 196. Each “X” in each of SEQ ID NOs: 196 and 198 may be mutated as described or may be the identity of the amino acid at the corresponding position in wild-type APOBEC3A (SEQ ID NO:3). Where “X” is not defined, it may be any amino acid.

In one embodiment, examples of ACDs having one or more stability-enhancing alteration and optionally one or more selectivity-enhancing substitution mutation are shown at SEQ ID NO:197, where the position number designation is functionally equivalent to the position in a wild-type APOBEC3A (SEQ ID NO:3) and “X” is an amino acid having the same or a different identity from the wild-type amino acid at that position, or a deletion as noted. Where “X” is not defined, it may be any amino acid. As is shown in SEQ ID NO:197, numerous amino acids may be mutated to enhance the stability and/or the selectivity of the ACD. While some mutations may be additive, some may be sufficient independently, depending on the desired activity of the ACD. Thus, the mutations indicated in SEQ ID NO: 197 are inclusive of many stability- and/or selectivity-enhancing mutations that may be made individually or in combination. SEQ ID NO: 199 is the sequence of one example of an ACD that preferentially deaminates unmodified cytosine shown with the numerous optional stability and selectivity mutations of SEQ ID NO: 197. Each “X” in each of SEQ ID NOs: 197 and 199 may be mutated as described or may be the identity of the amino acid at the corresponding position in wild-type APOBEC3A (SEQ ID NO:3). Where “X” is not defined, it may be any amino acid. The stability-enhancing mutations described in SEQ ID NOs: 196 and 198 are described further in Examples 6-8.

Each of SEQ ID NOs: 196-199 include an “X” for each position evaluated for a stability-enhancing alteration. An ACD of the present disclosure can include 1 or more, 2 or more, 3 or more, 4 or more, 5 or more, 6 or more, 7 or more, 8 or more, 9 or more, 10 or more, 11 or more, 12 or more, 13 or more, or 14 or more substitution alterations in any one of SEQ ID NOs: 196 or 197. An ACD of the present disclosure that preferentially deaminates unmodified cytosine can include 1 or more, 2 or more, 3 or more, 4 or more, 5 or more, 6 or more, 7 or more, 8 or more, 9 or more, 10 or more, 11 or more, 12 or more, 13 or more, or 14 or more substitution alterations in any one of SEQ ID NOs: 198 or 199.

SEQ ID NOs: 69-75, 76, 84, 85, 90, 93, 300-306, 308-369, 373, 376-378. 380-395, 397-486, 488-579, 582-672, 674-861, 864, 866, 872, 873, 877, 880-900, 902, 903, 905, 906, 908, 911, 915-917, 919-927, 929, 930, 932-937, 940, 941, 943-966, 968-977, 979-985, 987-990, 992-997, 999-1004, 1008-1013, 1016, 1018-1025, 1027-1029, 1031, 1033, 1035-1050, 1052-1057, 1059-1062, 1064, 1065, 1069, 1070, 1072-1090, 1092-1110, 1112-1155, 1157-1160, 1162-1165, 1167-1209, 1211-1235, and 1237 represent the amino acid sequences of 852 ACDs that were constructed and screened for activity. Sequences labeled “C” (SEQ ID NOs: 310-333) were found to preferentially deaminate unmodified cytosine. Sequences labeled “D” (SEQ ID NOs: 300-306, 308, 309) were found to be catalytically inactive. SEQ ID NOs: 69-75, 76, 84, 85, 90, 93 and sequences labeled “A” (SEQ ID NOs: 333-369, 373, 376-378. 380-395, 397-486, 488-579, 582-672, 674-861, 864, 866, 872, 873, 877, 880-900, 902, 903, 905, 906, 908, 911, 915-917, 919-927, 929, 930, 932-937, 940, 941, 943-966, 968-977, 979-985, 987-990, 992-997, 999-1004, 1008-1013, 1016, 1018-1025, 1027-1029, 1031, 1033, 1035-1050, 1052-1057, 1059-1062, 1064, 1065, 1069, 1070, 1072-1090, 1092-1110, 1112-1155, 1157-1160, 1162-1165, 1167-1209, 1211-1235, and 1237) were found to be catalytically active.

SEQ ID NOs: 1238-1241 represent consensus sequences of a multiple sequence alignment (MSA) of the 852 ACDs described in the preceding paragraph produced using Clustal Omega (McWilliam et al., 2013, Nucleic acids research 2013 July; 41 (Web Server issue): W597-600 doi: 10.1093/nar/gkt376, available on the world wide web at ebi.ac.uk/Tools/msa/clustalo/). SEQ ID NOs: 1238-1241 have 100%, 90%, 80%, and 70% consensus with SEQ ID NOs: 300-1237, respectively. X represents that there was no consensus residue at a location. Residues that were not identical but otherwise related (e.g., hydrophobic, small, polar, aromatic) in the consensus sequence are annotated within each consensus sequence. For the purposes of these consensus sequences, aliphatic residues include I, L, and V, alcohol residues include S and T, charged residues include D, E, H, K, and R, negatively charged residues include D and E, polar residues include C, D, E, H, K, N, Q, R, S, and T, positively charged residues include H, K, and R, small residues include A, C, D, G, N, P, S, T, and V, tiny residues include A, G, and S, and turnlike residues include A, C, D, E, G, H, K, N, Q, R, S, and T.

The person of ordinary skill in the art can confirm that the selectivity or the stability of an ACD disclosed herein is enhanced. For example, an ACD described herein can be constructed to include one or more selectivity-enhancing substitution mutations described herein and then evaluated for enhanced selectivity of C). A suitable assay is the SwaI-based assay (International Application Publication WO 2023/196572), where deamination of C by the ACD is compared to the same protein that does not include the same one or more selectivity-enhancing substitution mutations. A suitable assay is the fluorimetry assay described in International Application Publication WO 2023/196572.

An ACD described herein can include additional mutations. Typically, additional mutations do not unduly alter the activity of the altered cytidine deaminase. One or more additional mutations can be a conservative mutation. The specification discloses at FIG. 2 an alignment between a human APOBEC3A (SEQ ID NO:3, referred to as sp|P31941|1-199 in the figure), and other APOBEC3A proteins from other primates. The identical amino acids are marked with an “*” (asterisk), a “:” (colon) indicates conservation between amino acids of strongly similar properties, and a “.” (period) indicates conservation between groups of weakly similar properties. The skilled person would expect that many conservative substitutions in the areas that are identical or similar between the protein in the alignment would be likely to result in an active protein. Likewise, the skilled person would expect that many non-conservative substitutions in the areas that are identical or similar between the proteins in the alignment would be more likely to result in an inactive protein. Moreover, the location of the highly conserved spatial arrangement of the catalytic center residues of the ZDD motif is disclosed. The ZDD motif includes the H and two C residues which are believed to coordinate a Zn atom and the E residue which polarizes a water molecule near the Zn-atom for catalysis. As discussed herein the active site includes conserved residues such as tryptophan at position 98, serine or threonine at position 99, arginine at position 28, histidine, asparagine, or arginine at position 29, serine or threonine, preferably threonine, at position 31, asparagine or aspartic acid at position 57, tyrosine or phenylalanine at position 130, asparagine or tyrosine at position 131, asparagine, tyrosine, or phenylalanine, preferably tyrosine, at position 132, and arginine or lysine at position 189 of SEQ ID NO: 3 (Kouno et al., 2017, Nat. Comm, 8:15024, DOI: 10.1038/ncomms15024).

Dimerized ACDs (dACDs)

While APOBEC3A is typically found as a monomer, some variants form noncovalently associated dimers naturally in solution. Human APOBEC3A can form a homodimer via association of the so-called “dimerization interface” of the protein. (Bohn et al., 2015, Structure, 23 (5): 903-911). The purpose of dimerization has not been explicitly elucidated, but it is thought to be related to separation of binding and catalysis functions to improve target specificity. (Bohn et al.). The inventors herein demonstrated that dimerization of an ACD to form a dimerized ACD (dACD) improved enzyme stability and, resultantly, processivity.

As described herein, the introduction of a selectivity-enhancing mutation, such as a mutation to a position functionally equivalent to D133, was found to reduce the thermal melting point of ACDs that also included a substitution mutation at Y130, Y132, or both Y130 and Y132. Stability of ACDs is considered desirable because of their use in workflows that denature double-stranded DNA, often via increasing temperature, salt concentration, or chemical denaturant concentration, or a combination thereof.

The inventors found both modifications of additional amino acids that increase the stability of APOBEC proteins, and, in addition, that dimerization of ACDs increased the stability and the processivity of the enzyme. In addition, dimerization of a protein such as an ACD can increase the apparent local concentration of the enzyme, potentially increasing substrate turnover. The inventors found that producing dimerized altered cytidine deaminases provided beneficial properties over monomeric altered cytidine deaminases, as detailed more herein. The experimental data were obtained using ACDs having a 5mC-preferring activity; however, these data are applicable to ACDs having C-preferring activity.

Provided herein are dimeric altered cytidine deaminases (dACDs). A dACD includes a first protein and a second protein, wherein at least one of the first protein and the second protein is an altered cytidine deaminase. In some embodiments, the first protein is an altered cytidine deaminase, and the second protein is an altered cytidine deaminase. In some embodiments, the first protein is an altered cytidine deaminase, and the second protein is a wild-type cytidine deaminase. In some embodiments, the first protein is a wild-type cytidine deaminase, and the second protein is an altered cytidine deaminase.

The term “dimer” includes molecules which contain more than one protein linked together. The more than one protein can be joined by disulfide bonds, ionic bonds, or hydrophobic interactions, or complexes of proteins that are joined together, covalently or noncovalently, as dimers. The term protein refers to a linear organic polymer comprising a large number of amino-acid residues bonded together in a chain, forming part of (or the whole of) a protein molecule. The terms polypeptide, peptide, and oligopeptide are all encompassed within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

Typically, the first protein and the second protein are closely associated, and in one embodiment are covalently attached. Methods of covalently attaching a first and a second protein are described in greater detail herein. In some embodiments, however, the first protein and the second protein are associated via a strong noncovalent interaction such that they form a dimer in solution. In one such embodiment, the noncovalent interaction may be an interaction in which the dimer has a K_{D} of at most 1 μM. The “first protein” of a dimeric protein typically refers to the N-terminal protein, and the “second protein” of a dimeric protein typically refers to the C-terminal protein.

Briefly, a dACD may include one or two C-preferring ACDs having one or more selectivity-enhancing alteration described herein. In one embodiment, one or both of the ACDs have the amino acid sequence of SEQ ID NO:78. A C-preferring ACD can further include one or more stability-enhancing alterations described herein. Different combinations of ACDs may be advantageous in different embodiments depending on the desired catalytic activity and reaction conditions. Various combinations of exemplary dACDs are described in greater detail herein.

In some embodiments, the dACD is a homodimer, meaning that the first altered cytidine deaminase has an amino acid sequence that is identical to the amino acid sequence of the second altered cytidine deaminase. The amino acid sequence of an altered cytidine deaminase does not include domains such as linkers and purification tags (e.g., 6×His). For example, a dACD including, from N-terminus to C-terminus, the amino acid sequence of SEQ ID NO:78, where the X at position 130 is L; the X at position 132 is H; and the X at 133 is W, an amino acid linker (e.g., (G₄S)₂ linker, SEQ ID NO:267), the amino acid sequence of SEQ ID NO:78, where the X at position 130 is L; the X at position 132 is H; and the X at 133 is W, and a 6×His tag (i.e., HHHHHH, SEQ ID NO:220) would be considered to be a homodimeric ACD.

In some embodiments, the dACD is a heterodimer. A heterodimer is a dimer comprising a first altered cytidine deaminase having an amino acid sequence that is not identical to a second altered cytidine deaminase.

In some embodiments, a heterodimeric ACD includes a first altered cytidine deaminase and a second altered cytidine deaminase, wherein the first altered cytidine deaminase and the second altered cytidine deaminase have the same selectivity, e.g., both are C-preferring ACDs. As described herein, different amino acid sequences may give rise to the same substrate selectivity, particularly when the differences in amino acid sequence are in regions not related to substrate selectivity (e.g., regions related to stability). As is described herein, dimerization of an altered cytidine deaminase having a particular substrate preference may improve selectivity for that substrate. Additionally or alternatively, dimerization of an altered cytidine deaminase having a particular substrate preference may improve processivity for that substrate. In some embodiments, both of the ACDs of the dACD include one or more substitution mutations that modify the substrate specificity of the ACD (e.g., substitution mutations at positions functionally equivalent to Y130, Y132, and/or D133). The first ACD and the second ACD may include the same mutations or different mutations.

Alternatively, it may be desirable to provide a dimerized altered cytidine deaminase (dACD) wherein each cytidine deaminase has a different substrate preference. In some embodiments, a dACD may include an ACD described herein and wild-type cytidine deaminase. The ACD may have an engineered substrate preference, such as a preference for C, and the wild-type cytidine deaminase may have a wild-type substrate preference, such as a preference for both unmodified C and modified C, such as 5mC.

Interestingly, the inventors have found that in some embodiments, it is desirable for a dACD to include a catalytically inactive ACD. A catalytically inactive ACD may include any number of mutations in addition to one or more mutations to the active site of the enzyme. One example of a catalytically inactivating mutation is glutamate 72 to alanine (E72A), cysteine 101 to alanine (C101A), cysteine 106 to alanine (C106A), or a combination thereof.

In some embodiments, a dACD includes an ACD having one or more mutations to the dimerization interface. The dimerization interface may include positions which may interact between the two dimers. For example, a possible mutation to the dimerization interface includes mutation to a position functionally equivalent to H16, R28, K30, H56, or K60. Examples of mutations to the dimerization interface are listed in Table 9 below.

TABLE 9
Dimerization interface mutants.

| Residue | Mutation |
|---|---|
| H16 | A, L, W, D |
| R28 | A, V |
| K30 | A |
| H56 | A, R |
| K60 | A, P, G, R |

Using any method of protein conjugation or co-translation, one primary result of protein dimerization as it is described herein is the increased proximity between two altered cytidine deaminases. The distance between the two ACDs in the dimers described herein is typically less than 100 angstrom (Å), less than 90 Å, less than 80 Å, less than 70 Å, less than 60 Å, less than 50 Å, or less than 40 Å.

In embodiments wherein the first ACD and the second ACD are not covalently attached, formation of a dimer may be confirmed using molecular techniques to determine protein size, such as circular dichroism, and non-denaturing PAGE. A dACD will have a larger molecular weight than a monomeric ACD. A dACD will typically have a larger molecular diameter than a monomeric ACD.

A dACD of the present disclosure may be identified by its function. For example, a dACD of the present disclosure may exhibit greater thermostability than a comparable monomeric ACD. As it is used herein, a “comparable” monomeric ACD refers to an ACD having the amino acid sequence of one (or both, in the case of homodimers) of the cytidine deaminases of the dACD. In some embodiments, a dACD of the present disclosure has an increased thermal melting point than a comparable monomeric ACD. A dACD may have a melting temperature that is at least 1° C., at least 2° C., at least 3° C., at least 4° C., at least 5° C., or at least 6° C. compared to a monomeric ACD. A suitable method of determining the melting temperature of an ACD described herein is by fluorimetry.

In some embodiments, the dACD has a greater substrate preference as compared to a monomeric ACD.

While many possible heterodimeric altered cytidine deaminases are possible, several variants of interest will be prepared and measured. Table 10 describes dACD variants prepared in the backbone of SEQ ID NO:75 (see Example 11). The N-terminal ACD is catalytically inactive, either via introduction of E72 to A or via another inactivating mutation.

Table 11 describes dACD variants prepared in the backbone of SEQ ID NO:75. The C-terminal ACD is catalytically inactive, either via introduction of E72 to A or via another inactivating mutation.

It is understood that the active ACD using in Tables 10 and 11 was SEQ ID NO: 75, and it is contemplated that the heterodimers or homodimers can be made using any of the active ACDs described herein and that the description is not limited to just the active ACDs described in the Tables or Examples.

TABLE 10
Examples of heterodimeric altered cytidine
deaminases with an inactive N-terminus.

| N-term (inactive) | C-term (active) |
|---|---|
| A59/K60/61-68 | SEQ ID NO: 75 |
| Δ26-27 | SEQ ID NO: 75 |
| Δ104-105 | SEQ ID NO: 75 |
| A59/K60/61-68, Δ26-27 | SEQ ID NO: 75 |
| A59/K60/61-68, Δ104-105 | SEQ ID NO: 75 |
| A59/K60/61-68, Δ26-27, Δ104-105 | SEQ ID NO: 75 |
| Δ26-27, Δ104-105 | SEQ ID NO: 75 |
| Δ25-26 | SEQ ID NO: 75 |
| A59/K60/61-68, Δ25-26 | SEQ ID NO: 75 |
| A59/K60/61-68, Δ25-26, Δ104-105 | SEQ ID NO: 75 |
| Δ25-26, Δ104-105 | SEQ ID NO: 75 |
| Δ27-28 | SEQ ID NO: 75 |
| A59/K60/61-68, Δ27-28 | SEQ ID NO: 75 |
| A59/K60/61-68, Δ27-28, Δ104-105 | SEQ ID NO: 75 |
| Δ27-28, Δ104-105 | SEQ ID NO: 75 |
| Δ28-29 | SEQ ID NO: 75 |
| A59/K60/61-68, Δ28-29 | SEQ ID NO: 75 |
| A59/K60/61-68, Δ28-29, Δ104-105 | SEQ ID NO: 75 |
| Δ28-29, Δ104-105 | SEQ ID NO: 75 |
| Δ29-30 | SEQ ID NO: 75 |
| A59/K60/61-68, Δ29-30 | SEQ ID NO: 75 |
| A59/K60/61-68, Δ29-30, Δ104-105 | SEQ ID NO: 75 |
| Δ29-30, Δ104-105 | SEQ ID NO: 75 |
| Σ24-31: VRWAKGRRE | SEQ ID NO: 75 |
| Σ24-31: EPWRGRHE | SEQ ID NO: 75 |
| Σ24-39: KRRLRRPYYPRKAL | SEQ ID NO: 75 |
| Σ24-31: VRWAKGRRE, A59/K60/61-68, | SEQ ID NO: 75 |
| Δ104-105 | |
| Σ24-31: EPWRGRHE, A59/K60/61-68, | SEQ ID NO: 75 |
| Δ104-105 | |
| Σ24-39: KRRLRRPYYPRKAL, | SEQ ID NO: 75 |
| A59/K60/61-68, Δ104-105 | |
| Σ24-31: VRWAKGRRE, Δ104-105 | SEQ ID NO: 75 |
| Σ24-31: EPWRGRHE, Δ104-105 | SEQ ID NO: 75 |
| Σ24-39: KRRLRRPYYPRKAL, Δ104- | SEQ ID NO: |
| 105 | |

* A59/K60/61-68 = Reversion of A59P/K60R to A59 and K60, reintroduction of WT 61-68 loop.

TABLE 11
Examples of heterodimeric altered cysteine
deaminases with an inactive C-terminus.

| N-term (active) | C-term (inactive) |
|---|---|
| SEQ ID NO: 75 | A59/K60/61-68 |
| SEQ ID NO: 75 | Δ26-27 |
| SEQ ID NO: 75 | Δ104-105 |
| SEQ ID NO: 75 | A59/K60/61-68, Δ26-27 |
| SEQ ID NO: 75 | A59/K60/61-68, Δ104-105 |
| SEQ ID NO: 75 | A59/K60/61-68, Δ26-27, Δ104-105 |
| SEQ ID NO: 75 | Δ26-27, Δ104-105 |
| SEQ ID NO: 75 | Δ25-26 |
| SEQ ID NO: 75 | A59/K60/61-68, Δ25-26 |
| SEQ ID NO: 75 | A59/K60/61-68, Δ25-26, Δ104-105 |
| SEQ ID NO: 75 | Δ25-26, Δ104-105 |
| SEQ ID NO: 75 | Δ27-28 |
| SEQ ID NO: 75 | A59/K60/61-68, Δ27-28 |
| SEQ ID NO: 75 | A59/K60/61-68, Δ27-28, Δ104-105 |
| SEQ ID NO: 75 | Δ27-28, Δ104-105 |
| SEQ ID NO: 75 | Δ28-29 |
| SEQ ID NO: 75 | A59/K60/61-68, Δ28-29 |
| SEQ ID NO: 75 | A59/K60/61-68, Δ28-29, Δ104-105 |
| SEQ ID NO: 75 | Δ28-29, Δ104-105 |
| SEQ ID NO: 75 | Δ29-30 |
| SEQ ID NO: 75 | A59/K60/61-68, Δ29-30 |
| SEQ ID NO: 75 | A59/K60/61-68, Δ29-30, Δ104-105 |
| SEQ ID NO: 75 | Δ29-30, Δ104-105 |
| SEQ ID NO: 75 | Σ24-31: VRWAKGRRE |
| SEQ ID NO: 75 | Σ24-31: EPWRGRHE |
| SEQ ID NO: 75 | Σ24-39: KRRLRRPYYPRKAL |
| SEQ ID NO: 75 | Σ24-31: VRWAKGRRE, A59/K60/61- |
| | 68, Δ104-105 |
| SEQ ID NO: 75 | Σ24-31: EPWRGRHE, A59/K60/61-68, |
| | Δ104-105 |
| SEQ ID NO: 75 | Σ24-39: KRRLRRPYYPRKAL, |
| | A59/K60/61-68, Δ104-105 |
| SEQ ID NO: 75 | Σ24-31: VRWAKGRRE, Δ104-105 |
| SEQ ID NO: 75 | Σ24-31: EPWRGRHE, Δ104-105 |
| SEQ ID NO: 75 | Σ24-39: KRRLRRPYYPRKAL, Δ104- |
| | 105 |

* A59/K60/61-68 = Reversion of A59P/K60R to A59 and K60, reintroduction of WT 61-68 loop

### Fusion Proteins

In some embodiments, the dACD includes a fusion protein including an ACD. As it is used herein, a “fusion protein” describes a contiguously translated amino acid sequence including the amino acid sequences of more than one protein. In some embodiments, a fusion protein including an ACD includes a ACD covalently attached to a second cytidine deaminase. Typically, one or both of the cytidine deaminases are ACDs. However, in some embodiments, one of the cytidine deaminases is a wild-type cytidine deaminase.

A fusion protein including a dACD may include one or more linkers. In particular, a fusion protein may include an amino acid linker between the first cytidine deaminase and the second cytidine deaminase. The properties of the amino acid linker can be used to control the interaction between the first and second cytidine deaminases.

In some embodiments, the dACD includes a flexible linker. Typically, flexible amino acid linkers include amino acids having relatively small side chains.

Flexible linkers can provide a certain degree of movement or interaction giving the two ACD domains flexibility. Regarding the amino acid composition of the linkers, peptides are selected that do not interfere with the dimerization of the two polypeptides. For example, linkers comprising glycine and serine residues generally provide protease resistance. The amino acid sequence of the linkers can be optimized, for example, by phage-display methods.

Flexible linkers most commonly can be composed of small, non-polar (e.g., Gly) or polar (e.g., Ser or Thr) amino acids. A flexible linker can have sequences consisting primarily of stretches of Gly and Ser residues (“GS” linker). A non-limiting example of a flexible linker can have the sequence of (G₄S)n (SEQ ID NO:261), (SG₄)n (SEQ ID NO:262), G₄ (SG₄)n (G₄(SG₄)₁ shown in SEQ ID NO:263), wherein n can be 1-10, preferably wherein n is 1, 2, 3, 4, 5, 6, 7, 8, 9, or 10. By adjusting the copy number “n”, the length of this exemplary GS linker can be optimized to achieve appropriate separation of functional domains, or to maintain necessary inter-domain interactions. Other GS linkers include, but are not limited to, (GGSGGS)n (SEQ ID NO: 260), (GGSG) n, or (GGSGG)n (SEQ ID NO:219), wherein n is 1-10, preferably wherein n can be 1, 2, 3, 4, 5, 6, 7, 8, 9, or 10.

Besides GS linkers, other flexible linkers can be utilized for dimers. In some embodiment, a flexible linker can have the sequence of (Gly) n, wherein n can be 6, 7, or 8. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility. Thus, other suitable linkers may include, for example, GERP/GEKP/GQRP/GQKP or those disclosed in WO99/45132. For example, other linkers include, but are not limited to, GSPGSSSSGS (SEQ ID NO: 235), DPGGGGSGGGGSNPGS (SEQ ID NO:236), GGGGSGGGGSGSDPGS (SEQ ID NO: 237), DPGSGGGGSGGGGSGS (SEQ ID NO:238), GGGGSGGGGSGGGGSDPGS (SEQ ID NO: 239), DPGSGGGGSGGGGSGGGGS (SEQ ID NO:240), DPGSGSVPLGSGSNPGS (SEQ ID NO:241), DPGSGGSVPLGSGGSNPGS (SEQ ID NO:242), DPGVLEREDKPTTSKPNPGS (SEQ ID NO:243), DPGVLEREDVPTTSYPNPGS (SEQ ID NO: 244), DPGVLEREDKVTTSKYNPGS (SEQ ID NO:245), DPVLEREDKVTTSKNPGS (SEQ ID NO:246), DIEGRMD (SEQ ID NO:247), GEGKSSGSGSESKAS (SEQ ID NO: 248), GSTSGSGKPGSGEGSTKG (SEQ ID NO:249), GGGGSGGGGS (SEQ ID NO:267), SGGGGSGGGG (SEQ ID NO:250), GGGGSGGGGSGGGG (SEQ ID NO:251), GSPGSSSSGS (SEQ ID NO:235), GGGGSGGGGSGGGGSGGGGS (SEQ ID NO:253), GSGSGNGS (SEQ ID NO: 254), GGSGSGSG (SEQ ID NO:255), GGSGSG (SEQ ID NO:256), GGSG, GGSGNGSG (SEQ ID NO:257), GGNGSGSG (SEQ ID NO:258), and GGNGSGA(EAAAK)₄ALEA(EAAAK)₄A, (EAAAK)n (SEQ ID NO:259), n=1-6, among others.

In some embodiments, the protein linker includes the amino acid sequence:

(SEQ ID NO: 266)
DSGGSSGGSSGSETPGTSESATPESSGGSSGGS.

In some embodiments, the protein linker includes the amino acid sequence:

(SEQ ID NO: 267)
GGGGSGGGGS.

In some embodiments, the protein linker includes the amino acid sequence:

(SEQ ID NO: 268)
KESGSVSSEQLAQFRSLD.

In some embodiments, the protein linker includes the amino acid sequence:

(SEQ ID NO: 264)
EGKSSGSGESKST.

In some embodiments, the protein linker includes the amino acid sequence:

(SEQ ID NO: 269)
GSAGSAAGSGEF.

In some embodiments, the protein linker is a rigid linker. Rigid linkers may advantageously improve catalytic activity in some embodiments. Some rigid linkers form alpha helices. An example of a rigid linker that forms an alpha helix is the amino acid sequence A(EAAAK)ₙ, wherein n is typically 2, 3, 4, or 5 (SEQ ID NO:252). Some rigid linkers include a high percent composition of proline.

A fusion protein may include one or more additional amino acid sequences to confer additional properties to the fusion protein. For example, a fusion protein may include an affinity tag for protein purification, such as a His-tag (e.g., a 6×His, SEQ ID NO:220) or a FLAG™ tag (SEQ ID NO:218).

### Bioconjugation Protein Domains

In some embodiments, the fusion protein includes a first protein including a first dimerization domain and a first altered cytidine deaminase and a second protein including a second dimerization domain and a second altered cytidine deaminase.

In some embodiments, the dACD includes a first fusion protein including a first ACD including a first protein dimerization domain. The dACD may include a second fusion protein including a second ACD including a second protein dimerization domain.

The dimerization domain may include the proteins of the SpyCatcher/SpyTag™ (SEQ ID NOs: 226-227) dimerization pair. (Zakeri et al. 2012, Proc Natl Acad Sci 109 (12) E690-E697). To use this dimerization pair, the first ACD can be attached to SpyCatcher and the second ACD can be attached to SpyTag. Preferably, the first ACD is provided as a first fusion protein with SpyCatcher and the second ACD is provided as a second fusion protein with SpyTag. When the first fusion protein and the second fusion protein are mixed under physiological conditions, SpyTag and SpyCatcher form an isopeptide bond, covalently linking the first ACD to the second ACD. Advantageously, SpyTag and SpyCatcher may be fused to either terminus or between the termini of a protein, such as an ACD. Similar systems of proteins that form a strong covalent bond and can be used to create a covalent dimer of two proteins such as ACDs are known to the art. Examples of such proteins include the SnoopCatcher/SnoopTag™ system (SEQ ID NOs: 228-229).

Similar bioconjugation pairs exist that do not form covalent bonds, but strongly associate two proteins via their interaction. Examples of such bioconjugation pairs include GFP1-10 (SEQ ID NO:230) and GFP11 (SEQ ID NO:231), LgBit/HiBit™ (SEQ ID NOs: 232-233), leucine zippers (SEQ ID NO:234), and the Avi-Tag™ (SEQ ID NO:212)

### Chemical Linkage

In some embodiments, the dACD includes more than one cytidine deaminase attached via a chemical linkage. In some such embodiments, a first protein may include a first cytidine deaminase and a second protein may include a second cytidine deaminase. The first protein may be expressed separately from the second protein. One or both of the first cytidine deaminase and the second cytidine deaminase may include an ACD. Methods of conjugating a first cytidine deaminase and a second cytidine deaminase to form a dACD are described in greater detail herein.

### Polynucleotides Encoding ACDs

ACDs described herein also may be identified in terms of the polynucleotide that encodes the protein. Thus, this disclosure provides polynucleotides that encode an ACD described herein or hybridize, under standard hybridization conditions, to a polynucleotide that encodes an ACD described herein, and the complements of such polynucleotide sequences.

A polynucleotide as described herein can include any polynucleotide that encodes an ACD of the present disclosure. Thus, the nucleotide sequence of the polynucleotide may be deduced from the amino acid sequence that is to be encoded by the polynucleotide. An ACD can be encoded by multiple codons, and certain translation systems (e.g., prokaryotic or eukaryotic cells) often exhibit codon bias, e.g., different organisms often prefer one of the several synonymous codons that encode the same amino acid. As such, polynucleotides presented herein are optionally “codon optimized,” meaning that the polynucleotides are synthesized to include codons that are preferred by the particular translation system being employed to express the protein. For example, when it is desirable to express the protein in a bacterial cell (or even a particular strain of bacteria), the polynucleotide can be synthesized to include codons most frequently found in the genome of that bacterial cell, for efficient expression of the ACD. A similar strategy can be employed when it is desirable to express the ACD in a eukaryotic cell, e.g., the nucleic acid can include codons preferred by that eukaryotic cell.

A polynucleotide described herein may also, advantageously, be included in a suitable expression vector to express the ACD encoded therefrom in a suitable host. Incorporation of cloned DNA into a suitable expression vector for subsequent transformation of a host cell and subsequent selection of the transformed cells is well known to those skilled in the art. Suitable host cells include, but are not limited to, E. coli and S. cerevisiae.

Such an expression vector includes a vector having a polynucleotide described herein operably linked to heterologous regulatory sequences, such as promoter regions, that are capable of effecting expression of said DNA fragments. The term “operably linked” refers to a juxtaposition wherein the components described are in a relationship permitting them to function in their intended manner. Such vectors may be transformed into a suitable host cell to provide for the expression of an altered cytidine deaminase.

The nucleic acid molecule may encode a mature protein or a protein having a pro-sequence, including that encoding a leader sequence on the preprotein which is then cleaved by the host cell to form a mature protein. The vectors may be, for example, plasmid, virus or phage vectors provided with an origin of replication, and optionally a promoter for the expression of said nucleotide and optionally a regulator of the promoter. The vectors may contain one or more selectable markers, such as, for example, an antibiotic resistance gene.

Regulatory elements required for expression include promoter sequences to bind RNA polymerase and to direct an appropriate level of transcription initiation and also translation initiation sequences for ribosome binding. For example, a bacterial expression vector may include a promoter such as the lac promoter and for translation initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or be assembled from the sequences described by methods well known in the art.

Transcription of DNA encoding an ACD may be optimized by including an enhancer sequence in the vector. Enhancers are cis-acting elements of DNA that act on a promoter to increase the level of transcription. Vectors will also generally include origins of replication in addition to the selectable markers.

### Making and Isolating ACDs

Generally, polynucleotides encoding an ACD as presented herein can be made by cloning, recombination, in vitro synthesis, in vitro amplification and/or other available methods. A variety of recombinant methods can be used for expressing an expression vector that encodes an ACD presented herein. Methods for making recombinant polynucleotides, expression, and isolation of expressed products are well known and described in the art.

Polynucleotides encoding wild type cytidine deaminases can be obtained from a source and subjected to mutagenesis to introduce one or more substitution mutations described herein. In general, any available mutagenesis procedure can be used for making an ACD described herein. Procedures that can be used include, but are not limited to: site-directed point mutagenesis, in vitro or in vivo homologous recombination, oligonucleotide-directed mutagenesis, mutagenesis by total gene synthesis, and many others known to persons skilled in the art.

Additional useful references for mutation, recombinant, and in vitro nucleic acid manipulation methods (including cloning, expression, PCR, and the like) include Berger and Kimmel, Guide to Molecular Cloning Techniques, Methods in Enzymology volume 152 Academic Press, Inc., San Diego, Calif. (Berger); Kaufman et al. (2003) Handbook of Molecular and Cellular Methods in Biology and Medicine Second Edition Ceske (ed) CRC Press (Kaufman); The Nucleic Acid Protocols Handbook Ralph Rapley (ed) (2000) Cold Spring Harbor, Humana Press Inc (Rapley); Chen et al. (ed) PCR Cloning Protocols, Second Edition (Methods in Molecular Biology, volume 192) Humana Press; and in Viljoen et al. (2005) Molecular Diagnostic PCR Handbook Springer, ISBN 1402034032.

In addition, many kits are commercially available for the purification of plasmids or other relevant nucleic acids from cells. An isolated polynucleotide can be further manipulated to produce other polynucleotides, used to transfect or transform cells, incorporated into related vectors and introduced into cells for expression, and/or the like. Typical cloning vectors contain transcription and translation terminators, transcription and translation initiation sequences, and promoters useful for regulation of the expression of the particular target nucleic acid. The vectors optionally include generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Other useful references, e.g., for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla. Construction of vectors containing a nucleic acid encoding an ACD described herein employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994).

A variety of protein isolation and detection methods are known and can be used to isolate an ACD, e.g., from recombinant cultures of cells expressing the recombinant cytidine deaminase presented herein. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

An ACD protein or polynucleotide can be isolated. An “isolated” protein or polynucleotide is one that has been removed from a cell. For instance, an isolated protein is a polypeptide that has been removed from the cytoplasm or from the membrane of a cell, and many of the proteins, nucleic acids, and other cellular material of its natural environment are no longer present. Proteins that are produced outside of a cell, e.g., through chemical or recombinant means, are considered to be isolated by definition, as they were never present in a cell.

### Methods of Use

The ACDs provided by the present disclosure can be easily integrated into essentially any application for identifying modified cytosines. For instance, ACDs can be integrated into applications that include sequencing library preparation. Examples of sequencing library preparation include, but are not limited to, whole genome, accessible (e.g., ATAC), conformational state (e.g., HiC), and reduced representation bisulfite sequencing (RRBS). It can be particularly useful in essentially any application using low input DNA or RNA such as, but not limited to, single cell combinatorial indexing (sci) methods like sci-WGS-seq, sci-MET-seq, and sci-ATAC-seq, sci-RNA-seq, and cell free DNA-based methods. Specific applications include, but are not limited to, identifying one or more patterns of cytosine modification such as determining methylation on CpG islands and reduced representation bisulfite sequencing (RRBS); variant calling, including SNV/indel, copy number variation (CNV), short tandem repeats (STR), and structural variants (SV); detecting differentially methylated regions (DMRs); measuring methylation at promoters; and detecting tumor DNA (International Application No. PCT/US2023/017846.

The ACDs provided by the present disclosure can be easily integrated into essentially any application that includes locus-specific methylation profiling. Typical locus-specific detection of epigenetic methylated cytosines, such as 5mC, require the use of 5mC-specific antibodies, or multi-step chemical or chemoenzymatic transformations that lead to deamination of C or 5mC to U/T to enable differentiation of the two C-isoforms. When combined with various in vitro detection methodologies, these approaches can be strong approaches to detect 5mC at defined loci. However, these methods can be confounded by antibody cross-reactivity and stability, or the toxicity and complex workflows required by chemical and chemoenzymatic approaches. Use of an ACD described herein in a single-step enzymatic deamination protocol permits selective conversion of C to U that is compatible with a number of in vitro diagnostic modalities, resulting in locus-specific detection of modified cytosines, including 5mC and 5hmC.

Instead of using destructive methods for identifying methylated cytosines, integrating the cytidine deaminases provided by the present disclosure into methods for identifying modified cytosines, such as sequencing library production and locus-specific methylation profiling, results in the more efficient enzyme-catalyzed conversion of modified cytosines during generation of target nucleic acids, thereby permitting better sequencing data and better retention of genetic information, which is demonstrated by high variant calling performance (International Application No. PCT/US2023/017846). Furthermore, as an enzymatic method for conversion, the use of the ACDs enables high coverage uniformity and low sample damage, which results in lower nucleic acid input requirements. A multitude of sequencing library methods are known to a skilled person that can be used in the construction of whole-genome or targeted libraries.

In general, methods for using an ACD of the present disclosure include contacting target nucleic acids, e.g., DNA or RNA, with the enzyme, under conditions suitable for conversion of modified cytidines. Because amplification of DNA does not preserve the modification status of cytidine (e.g., the methylation status of 5mC and 5hmC is not retained), use of an ACD typically occurs before amplification of target DNA. Target nucleic acids can be contacted with an ACD at essentially any time in a method before an amplification, provided the DNA is single-stranded. For instance, target nucleic acids can be contacted with an ACD while the nucleic acids are inside a fixed or unfixed cell or nucleus, after isolation of genomic or cell free DNA or mRNA, before or after fragmentation, or before or after tagmentation. The skilled person will recognize that target nucleic acids can be contacted with ACD after addition of a universal sequence and/or an adapter, provided the universal sequence and/or an adapter is not added by amplification.

A method for using an ACD can include the optional step of comparison of the treated target nucleic acid with an untreated nucleic acid or comparison of the treated target nucleic acid with a nucleic acid treated with a wild-type cytidine deaminase. For instance, in embodiments wherein the treated nucleic acid is sequenced, the sequence can be compared to a reference sequence thereby permitting easy identification of point mutations and inference of modified cytosines. Thus, in embodiments wherein an ACD having C-preferring deaminase activity (converts C to U at a greater rate than converting 5mC to T) is used, C will be converted to U, which will be read out as T upon sequencing, and 5mC will be read out as C. By comparing the sequencing reads of treated and untreated samples, a methylation level of each C position can be inferred quantitatively. In embodiments where the treated nucleic acid is not sequenced, the nucleic acid can be treated with an ACD and compared to the nucleic acid that is untreated, i.e., not contacted with an ACD. Here the read-out typically depends on the assay method, for instance when an amplification is used the relative amounts of amplification can be easily identified and the presence or absence of a 5mC or a pattern of cytosine modification at a predetermined sequence inferred (International Application No. PCT/US2023/017846).

Reaction conditions suitable for conversion of modified cytidines, such as conversion of C to uracil, by a cytidine deaminase described herein include, but are not limited to, a substrate of target nucleic acid that is single-stranded (ss) DNA or RNA suspected of including at least one modified cytidine, buffer, pH, temperature of the reaction, time of the reaction, and concentration of the ACD and/or ss DNA or RNA substrate. In one embodiment, double-stranded (ds) DNA can be denatured and exposed to an ACD. Methods for denaturing dsDNA are known and routine, and include heat treatment, chemical treatment, such as NaOH, formamide, DMSO, or N, N-dimethylformamide (DMF), or a combination thereof.

Target nucleic acids useful in the methods of the present disclosure are described herein. A modified cytidine present on a substrate single-stranded (ss) DNA or RNA includes, but is not limited to, 5-methyl cytosine (5mC), 5-hydroxymethyl cytosine (5hmC), 5-formyl cytosine (5fC), and 5-carboxy cytosine (5CaC). In one embodiment, the modified cytidine is 5-methyl cytosine. In one embodiment, the modified cytidine is 5-hydroxymethyl cytosine. Methods that use double stranded target DNA for generating a sequencing library can be modified to include denaturation to convert the double stranded target DNA to ssDNA. In some embodiments, dsDNA that is used in a tagmentation reaction or for adapter attachment can be denatured and then treated with an ACD. Conditions for denaturation are known and routine. In those embodiments where ssDNA is contacted with an ACD and subsequently used in a process that requires dsDNA, e.g., addition of universal adapters by tagmentation or ligation, the ssDNA can be converted to dsDNA using routine methods.

In some embodiments, target nucleic acids obtained from a subject can be treated with an ACD to result in converted nucleic acids, and a pattern of cytosine modification can be identified in the converted nucleic acids. The pattern of cytosine modification can optionally be compared with the pattern of cytosine modification in a reference nucleic acid. In embodiments where a pattern of cytosine modification correlates with a disease or condition, the method can be used in diagnostic or prognostic applications. For instance, the subject can have or be at risk of having a disease or condition, and the reference nucleic acid can be from a normal subject, e.g., a subject that does not have and is not at risk for the disease or condition. The pattern of cytosine modification can be associated with a disease or condition (e.g., the target nucleic acid can be a predetermined sequence), and identification in the subject of a pattern of cytosine modification associated with a disease or condition can indicate the subject has or is at risk of having the disease or condition. For instance, a pattern of cytosine modification can be linked in-cis to a coding region that is correlated with a disease or condition and identification of that pattern, or absence of that pattern, in the subject can be used for diagnosis or prognosis. In one embodiment, the coding region can be one that is transcriptionally active or transcriptionally inactive in a reference nucleic acid. The comparison of the converted nucleic acid to the reference nucleic acid can include determining if the pattern of cytosine modification of the converted nucleic acid indicates the coding region is transcriptionally active or transcriptionally inactive in the subject. When that coding region is associated with a disease or condition, the status of transcriptional activity can be used for diagnosis or prognosis.

Comparison of a pattern of cytosine modification in a subject can also be used in identifying changes in a pattern of cytosine modification in a subject over time. For instance, a subject can have a disease or conditions and is undergoing treatment, or a subject had a disease or condition and is cured (e.g., the subject was treated and no signs of the disease or condition are present) or in remission (e.g., the subject was treated and signs of the disease or condition are reduced). Target nucleic acids from the subject at different times, e.g., before treatment started, during treatment, after treatment is stopped, can be compared and a pattern of cytosine modification of a sequence, e.g., a predetermined sequence compared and used to determine the progress of a treatment or the status of the disease or condition in the subject.

Wild-type cytidine deaminases typically function at near-neutral pH, e.g., pH 7. ACDs described herein can have increased activity at below neutral pH. In some embodiments, the pH of a reaction that includes an ACD described herein can be no greater than pH 8, no greater than 7.8, no greater than 7.7, no greater than 7.6, no greater than 7.5, no greater than 7.4, no greater than 7.3, no greater than 7.2, no greater than 7.1, no greater than 7.0, no greater than pH 6.7, no greater than pH 6.5, no greater than pH 6.3, no greater than pH 6.1, no greater than pH 6.0. In some embodiments, the pH of a reaction that includes an ACD described herein can be at least pH 5.1, at least pH 5.3, at least pH 5.5, at least pH 5.7, at least pH 5.9, at least pH 6.1, at least pH 6.3, at least pH 6.5, at least pH 6.7, at least pH 6.9, at least pH 7.0, at least pH 7.1, at least pH 7.2, at least pH 7.3, at least pH 7.4, at least pH 7.5. In some embodiments, the pH of a reaction that includes an ACD described herein can be no greater than pH 7.5, no greater than pH 7.3, or no greater than pH 7.1. Examples of ranges of pH in a reaction include at least 6 to no greater than 8, at least 6.5 to no greater than 8, at least 7 to no greater than 8, or at least 6.9 to no greater than 7.6, e.g., pH of about 7.0 to about 7.5. The activity of an ACD to deaminate a 5mC oligonucleotide substrate can be an increased catalytic activity that is at least 10-fold greater, at least 50-fold greater, or at least 100-fold greater when comparing activity.

It is expected that an ACD can function in essentially any buffer. Examples of useful buffers include, but are not limited to: a citrate buffer, such as the citrate buffer available from ThermoFisher Scientific (Cat. No. #005000); sodium acetate buffer, Bis Tris-Propane HCl; and Tris-HCl Tris. Examples of other buffers include, but are not limited to, Bicine, DIPSO, glycylglycine, HEPES, imidazole, malonate, MES, MOPS, PB, phosphate, PIPES, SPG, succinate, TAPS, TAPSO, trincine. In some embodiments a reducing agent such as dithiothreitol (DTT), TCEP, can be present. In some embodiments a divalent cation may be included, for example, Zinc. In some embodiments, a divalent cation is not included.

A deamination reaction can occur at a temperature of 25° C. to 75° C., such as 37 or 50° C. Suitable temperature ranges include from about 37° C. to 75° C., alternatively about 42° C. to 75° C., alternatively about 48° C.-75° C., about 50° C. to 75° C., alternatively about 50° C. to 65° C., and any temperature or range between. Some ACDs described herein preferentially deaminate a modified cytosine to thymidine at a faster rate than deamination of cytosine to uracil. Thus, in some embodiments the time of reaction can be used to maximize the difference of deamination of modified cytosine versus deamination of cytosine. In one embodiment, the reaction can proceed for at least 15 minutes, at least 30 minutes, at least 45 minutes, at least 60 minutes, at least 90 minutes, at least 120 minutes, or at least 150 minutes, and for no greater than 15 minutes, no greater than 30 minutes, no greater than 45 minutes, no greater than 60 minutes, no greater than 90 minutes, no greater than 120 minutes, no greater than 150 minutes, or no greater than 180 minutes.

In one embodiment, a deamination reaction can include an ACD at a concentration from at least 0.05 micromolar (μM) to no greater than 5 μM. For instance, the concentration of the enzyme can be at least 0.05, at least 0.1 μM, at least 0.2 μM, at least 0.3 μM, at least 0.4 μM, or at least 0.5 μM, at least 0.6 μM, at least 0.7 μM, at least 0.8 μM, at least 0.9 μM, at least 1.0 μM and/or no greater than 5 μM, no greater than 4 μM, no greater than 3 μM, no greater than 2 μM, no greater than 1 μM, or 0.5 μM. In one embodiment, a deamination reaction can include nucleic acids at a concentration of at least 1 picomolar (pM) to no greater than 2 μM. For instance, the concentration of nucleic acids can be at least 1 pM, at least 3 pM, at least 6 pM, at least 10 pM, at least 100 pM, at least 1 nanomolar (nM), at least 40 nM, at least 400 nM, at least 500 nM, at least, 600 nM, at least 700 nM, at least 800 nM, at least 900 nM, or 1 μM, and/or no greater than 1 μM, no greater than 900 nM, no greater than 800 nM, no greater than 700 nM, no greater than 600 nM, no greater than 500 nM, no greater than 400 nM, no greater than 40 nM, no greater than 1 nM, no greater than 100 nM, no greater than 6 nM, or no greater than 3 nM.

The substrate of an ACD described herein can be a single stranded nucleic acid, such as single stranded DNA (ssDNA). ssDNA is susceptible to the formation of secondary structure such as hairpins, which can reduce accessibility of reaction sites to an ACD and increase the production of false positives. Accordingly, some embodiments a method of using an ACD can include the use of one or more denaturant to reduce the formation of secondary structure. Examples of denaturants include DMSO and betaine.

Stability-enhancing substitution mutations increase the thermal melting point of an ACD, and increased temperature optimums are highly desirable because it decreases DNA secondary structures by opening reaction sites that would be otherwise inaccessible due to secondary structure resulting in decreased false positive rate; stabilizes the enzyme in reaction conditions, which permits longer incubations and increased conversion; increases reaction kinetics, which allows for more tightly controlled conditions; and improved characteristics for commercialization, including increased shelf life, robustness in the assay, manufacturability, etc.

In one embodiment, a deamination reaction can include an RNAse. RNase A has been implicated in increasing activity of cytidine deaminases (Bransteitter et al., Proceedings of the National Academy of Sciences of the United States of America 100, no. 7 (2003): 4102-7. doi.org/10.1073/pnas.0730835100). When activity of an ACD of the present disclosure was determined in the presence of RNAse A the opposite was observed. When RNAse A was included in the reaction, an ACD having cytosine-defective deaminase activity (i.e., converts 5mC to T at a greater rate than converting C to U) had reduced activity, and the reduced activity was more pronounced for off-target cytosine deamination. Thus, RNAse A resulted in greater selectivity for deamination of 5mC compared to C. An RNAse A can be included in a deamination reaction at a concentration from at least 1 microgram/milliliter (ug/ml) to no greater than 20 μM. For instance, the concentration of RNAse A can be at least 1 ug/ml, at least 2 ug/ml, at least 3 ug/ml, at least 4 ug/ml, 5 ug/ml, 6 ug/ml, 7 ug/ml, 8 ug/ml, or 9 ug/ml, and/or no greater than 50 ug/ml, no greater than 40 ug/ml, no greater than 30 ug/ml, no greater than 20 ug/ml, no greater than 19 ug/ml, no greater than 18 ug/ml, no greater than 17 ug/ml, no greater than 16 ug/ml, no greater than 15 ug/ml, no greater than 14 ug/ml, no greater than 13 ug/ml, no greater than 12 ug/ml, or no greater than 11 ug/ml. In one embodiment, the concentration of RNAse A is from 2 ug/ml to 13 ug/ml, or from 5 ug/ml to 10 ug/ml.

### Target Nucleic Acids

The target nucleic acids contacted with an ACD and used in the methods, compositions, and kits provided herein may be essentially any nucleic acid of known or unknown sequence. Sequencing may result in determination of the sequence of the whole or a part of the target molecule. In one embodiment, target nucleic acids can be processed into templates suitable for amplification by the placement of universal amplification sequences, e.g., sequences present in a universal adaptor, at the ends of each target fragment.

Target nucleic acids are typically derived from primary nucleic acids present in a sample, such as a biological sample. The primary nucleic acids may originate as DNA or RNA. DNA primary nucleic acids may originate in double-stranded DNA (dsDNA) form (e.g., genomic DNA, genomic DNA fragments, cell-free DNA, and the like) from a sample or may originate in single-stranded form from a sample. RNA primary nucleic acids may be mRNA or non-coding RNA, e.g., microRNA or small interfering RNA. The precise sequence of the polynucleotide molecules from a primary nucleic acid sample is generally not material to the disclosure and may be known or unknown.

The primary nucleic acid molecules may represent the entire genetic complement of an organism, e.g., genomic DNA molecules which include both intron and exon sequences, as well as non-coding regulatory sequences such as promoter and enhancer sequences. The primary nucleic acid molecules may represent the entire genetic complement of specific cells of an organism, e.g., from tumor cells, where the genomic DNA molecules which include both intron and exon sequences, as well as non-coding regulatory sequences such as promoter and enhancer sequences. In one embodiment, particular subsets of genomic DNA can be used, such as, for example, particular chromosomes, DNA associated with open chromatin, DNA associated with closed chromatin, or one or more specific sequences such as a region of a specific gene (e.g., targeted sequencing). In one embodiment, the primary nucleic acid molecules may represent a particular subset of DNA, e.g., DNA having a specific sequence that anneals with a primer such as one used for targeted sequencing or target enrichment. In one embodiment, a particular subset of DNA can be used, such as cell-free DNA, which can include DNA of the subject including DNA from normal cells, DNA from diseased cells such as tumor cells, and/or DNA from fetal cells.

The primary nucleic acid molecules may represent the entire transcriptome of cells of an organism, e.g., mRNA molecules. The primary nucleic acid molecules may represent the entire transcriptome of specific cells of an organism, e.g., from tumor cells or for instance the cells of a tissue. In one embodiment, the primary nucleic acid molecules may represent a particular subset of mRNA, e.g., mRNA having a specific sequence that anneals with a primer such as one used for targeted sequencing or target enrichment.

A sample, such as a biological sample, can include nucleic acid molecules obtained from biopsies, tumors, scrapings, swabs, blood, mucus, urine, stool, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include cultured cells. In some embodiments, the sample can include nucleic acid molecules obtained from an animal such as a human or mammalian source. In another embodiment, the sample can include nucleic acid molecules obtained from a non-mammalian source such as a plant, bacteria, virus, or fungus. In some embodiments, the source of the nucleic acid molecules may be an archived or extinct sample or species.

Additional non-limiting examples of sources of biological samples can include whole organisms as well as a sample obtained from a patient. The biological sample can be obtained from any biological fluid or tissue and can be in a variety of forms, including fluid, e.g., liquid or gas, tissue, solid tissue, and preserved forms of such a fluid or tissue, such as dried, frozen, and fixed forms. The sample may be of any biological tissue, cells or fluid. Such samples include, but are not limited to, sputum, blood, serum, plasma, blood cells (e.g., white cells), ascitic fluid, urine, saliva, tears, sputum, vaginal fluid (discharge), washings obtained during a medical procedure (e.g., pelvic or other washings obtained during biopsy, endoscopy or surgery), tissue, nipple aspirate, core or fine needle biopsy samples, cell-containing body fluids, peritoneal fluid, and pleural fluid, or cells therefrom, and free floating nucleic acids such as cell-free circulating DNA. Biological samples may also include sections of tissues such as frozen or fixed sections taken for histological purposes or micro-dissected cells or extracellular parts thereof. In some embodiments, the sample can be a blood sample, such as, for example, a whole blood sample. In another example, the sample is an unprocessed dried blood spot (DBS) sample. In yet another example, the sample is a formalin-fixed paraffin-embedded (FFPE) sample. In yet another example, the sample is a saliva sample. In yet another example, the sample is a dried saliva spot (DSS) sample.

Exemplary biological samples from which target nucleic acids can be derived include, for example, those from a eukaryote, for instance a mammal, such as a rodent, mouse, rat, rabbit, guinea pig, ungulate, horse, sheep, pig, goat, cow, cat, dog, primate, human or non-human primate; a plant, such as Arabidopsis thaliana, corn, sorghum, oat, wheat, rice, canola, or soybean; an algae, such as Chlamydomonas reinhardtii; a nematode such as Caenorhabditis elegans; an insect, such as Drosophila melanogaster, mosquito, fruit fly, honey bee or spider; a fish, such as zebrafish; a reptile; an amphibian, such as a frog or Xenopus laevis; a Dictyostelium discoideum; a fungi, such as Pneumocystis carinii, Takifugu rubripes, yeast, Saccharamoyces cerevisiae, or Schizosaccharomyces pombe; or a protozoan such as Plasmodium falciparum. Target nucleic acids can also be derived from a prokaryote such as a bacterium, Escherichia coli, Staphylococcus or Mycoplasma pneumoniae; an archaeon; a virus such as Hepatitis C virus or human immunodeficiency virus; or a viroid. Target nucleic acids can be derived from a homogeneous culture or population of organisms described herein or alternatively from a collection of several different organisms, for example, in a community or ecosystem.

In some embodiments, a biological sample includes tissue that is processed to obtain the desired primary nucleic acids. In some embodiments, cells are used obtain the desired primary nucleic acids. In some embodiments, nuclei are used to obtain the desired primary nucleic acids. The method can further include dissociating cells, and/or isolating nuclei from cells. Methods for isolating cells and nuclei from tissue are available (WO 2019/236599).

In some embodiments, nucleic acids present in tissue, in cells, or in isolated nuclei can be processed depending on the desired read-out. For instance, nucleic acids can be fixed during processing, and useful fixation methods are available (WO 2019/236599). Fixation can be useful to preserve a sample or maintain contiguity of analytes from a sample, a cell, or a nucleus. Fixation methods preserve and stabilize tissue, cell, and nucleus morphology and architecture, inactivates proteolytic enzymes, strengthens samples, cells, and nuclei so they can withstand further processing and staining, and protects against contamination. Examples of methods where fixation can be useful include, but are not limited to, whole genome sequencing of isolated nuclei and chromosome conformation capture methods such as Hi-C. Common methods of fixation include perfusion, immersion, freezing, and drying (Srinivasan et al., Am J Pathol. 2002 December; 161 (6): 1961-1971.doi: 10.1016/S0002-9440 (10) 64472-0). In some embodiments such as whole genome sequencing, isolated nuclei can be processed to dissociate nucleosomes from DNA while leaving the nuclei intact, and methods for generating nucleosome-free nuclei are available (WO 2018/018008).

In some embodiments, primary nucleic acids in bulk, e.g., from a plurality of cells, can be used to produce a sequencing library as described herein. In other embodiments, individual cells or nuclei can be used as sources of primary nucleic acids to obtain sequence information from single cells and nuclei. Many different single cell library preparation methods are known in the art, including, but not limited to, Drop-seq, Seq-well, and single cell combinatorial indexing (“sci-”) methods. Companies providing single cell products and related technologies include, but are not limited to, Illumina, 10X Genomics, Takara Biosciences, BD Biosciences, Bio-Rad Laboratories, 1cellbio, Isoplexis, CellSee, NanoSelect, and Dolomite Bio. Sci-seq is a methodological framework that employs split-pool barcoding to uniquely label the nucleic acid contents of large numbers of single cells or nuclei. Typically, the number of nuclei or cells can be at least two. The upper limit is dependent on the practical limitations of equipment (e.g., multi-well plates, number of indexes) used in other steps of the methods as described herein. The number of nuclei or cells that can be used is not intended to be limiting and can number in the billions.

The target nucleic acids used in the methods and compositions of the present disclosure can be derived by fragmentation. Random fragmentation refers to the fragmentation of a polynucleotide molecule from a primary nucleic acid sample in a non-ordered fashion by enzymatic, chemical, or mechanical methods. Such fragmentation methods are known in the art and use standard methods (Sambrook and Russell, Molecular Cloning, A Laboratory Manual, third edition). Moreover, random fragmentation is designed to produce fragments irrespective of the sequence identity or position of nucleotides comprising and/or surrounding the break. In one embodiment, the random fragmentation is by mechanical means such as nebulization or sonication to produce fragments of about 50 base pairs in length to about 1500 base pairs in length, still more particularly 50-700 base pairs in length, yet more particularly 50-400 base pairs in length. Most particularly, the method is used to generate smaller fragments of from 50-150 base pairs in length.

Fragmentation of polynucleotide molecules by mechanical means (nebulization, sonication, and Hydroshear, for example) results in fragments with a heterogeneous mix of blunt and 3′- and 5′-overhanging ends. It is therefore desirable to repair the fragment ends using methods or kits (such as the Lucigen DNA terminator End Repair Kit) known in the art to generate ends that are optimal for insertion, for example, into blunt sites of cloning vectors. In a particular embodiment, the fragment ends of the population of nucleic acids are blunt ended. More particularly, the fragment ends are blunt ended and phosphorylated. The phosphate moiety can be introduced via enzymatic treatment, for example, using polynucleotide kinase.

In a particular embodiment, the target fragment sequences are prepared with single overhanging nucleotides by, for example, activity of certain types of DNA polymerase such as Taq polymerase or Klenow exo minus polymerase which has a non-template-dependent terminal transferase activity that adds a single deoxynucleotide, for example, deoxyadenosine (A) to the 3′ ends of a DNA molecule, for example, a PCR product. Such enzymes can be used to add a single nucleotide ‘A’ to the blunt ended 3′ terminus of each strand of the double-stranded target fragments. Thus, an ‘A’ could be added to the 3′ terminus of each end repaired strand of the double-stranded target fragments by reaction with Taq or Klenow exo minus polymerase, while the universal adapter polynucleotide construct could be a T-construct with a compatible ‘T’ overhang present on the 3′ terminus of each region of double-stranded nucleic acid of the universal adapter. This end modification also prevents self-ligation of both vector and target such that there is a bias towards formation of target nucleic acids having a universal adapter at each end.

In one embodiment, fragmentation can be accomplished using a process often referred to as tagmentation. Tagmentation uses a transposome complex and combines into a single step fragmentation and ligation to add universal adapters (WO 2016/130704). A transposome complex is a transposase bound to a transposase recognition site and can insert the transposase recognition site into a target nucleic acid in a process sometimes termed “tagmentation.” In some such insertion events, one strand of the transposase recognition site may be transferred into the target nucleic acid. Such a strand is referred to as a “transferred strand.” In one embodiment, a transposome complex includes a dimeric transposase having two subunits, and two non-contiguous transposon sequences. In another embodiment, a transposase includes a dimeric transposase having two subunits, and a contiguous transposon sequence.

Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35:785, 1983; Savilahti, H, et al., EMBO J., 14:4893, 1995). Tn5 Mosaic End (ME) sequences can also be used by a skilled artisan.

Examples of transposon sequences useful with the methods and compositions described herein are provided in U.S. Patent Application Pub. No. 2012/0208705, U.S. Patent Application Pub. No. 2012/0208724 and Int. Patent Application Pub. No. WO 2012/061832. In some embodiments, a transposon sequence includes a first transposase recognition site and a second transposase recognition site.

Some transposome complexes useful herein include a transposase having two transposon sequences. In some such embodiments, the two transposon sequences are not linked to one another, in other words, the transposon sequences are non-contiguous with one another. Examples of such transposomes are known in the art (see, for instance, U.S. Patent Application Pub. No. 2010/0120098).

In one embodiment, tagmentation is used to produce target nucleic acids that include different universal sequences at each end. This can be accomplished by using two types of transposome complexes, where each transposome complex includes a different nucleotide sequence that is part of the transferred strand.

A population of target nucleic acids can have an average strand length that is desired or appropriate for a particular application of the methods, compositions, or kits set forth herein. For example, the average strand length can be less than about 100,000 nucleotides, 50,000 nucleotides, 10,000 nucleotides, 5,000 nucleotides, 1,000 nucleotides, 500 nucleotides, 100 nucleotides, or 50 nucleotides. Alternatively or additionally, the average strand length can be greater than about 10 nucleotides, 50 nucleotides, 100 nucleotides, 500 nucleotides, 1,000 nucleotides, 5,000 nucleotides, 10,000 nucleotides, 50,000 nucleotides, or 100,000 nucleotides. The average strand length for a population of target nucleic acids can be in a range between a maximum and minimum value set forth herein. It will be understood that amplicons generated at an amplification site (or otherwise made or used herein) can have an average strand length that is in a range between an upper and lower limit selected from those exemplified above.

In some cases, a population of target nucleic acids can be produced under conditions or otherwise configured to have a maximum length for its members. For example, the maximum length for the members that are used in one or more steps of a method set forth herein or that are present in a particular composition can be less than 100,000 nucleotides, less than 50,000 nucleotides, less than 10,000 nucleotides, less than 5,000 nucleotides, less than 1,000 nucleotides, less than 500 nucleotides, less than 100 nucleotides, or less than 50 nucleotides. Alternatively or additionally, a population of target nucleic acids can be produced under conditions or otherwise configured to have a minimum length for its members. For example, the minimum length for the members that are used in one or more steps of a method set forth herein or that are present in a particular composition can be more than 10 nucleotides, more than 50 nucleotides, more than 100 nucleotides, more than 500 nucleotides, more than 1,000 nucleotides, more than 5,000 nucleotides, more than 10,000 nucleotides, more than 50,000 nucleotides, or more than 100,000 nucleotides. The maximum and minimum strand length for target nucleic acids in a population can be in a range between a maximum and minimum value set forth above. It will be understood that amplicons generated at an amplification site (or otherwise made or used herein) can have maximum and/or minimum strand lengths in a range between the upper and lower limits exemplified above.

In some embodiments, a sample can be enriched for sequences of interest, e.g., a predetermined sequence. For example, a subset of genes or regions of the genome are isolated and sequenced, or a subset of genes or regions of the genome are interrogated by other methods, such as a locus-specific in vitro diagnostic method. A predetermined sequence can be, for instance, one that can have a pattern of cytosine modification.

In some embodiments, target enrichment works by capturing genomic regions of interest by hybridization to target-specific probes that can be used to physically separate target DNA that has hybridized to bait probes from all other DNA in solution, which are then washed away. For example, some methods of enrichment use biotinylated probes, which are then isolated by magnetic pulldown with streptavidin-coated magnetic particles. In another example, some methods of enrichment use analyte arrays, also known as microarrays, that allow for the hybridization of predetermined sequences.

Enrichment can occur, for example, prior to treatment with ACD. In such embodiments, enriching a nucleic acid of interest, or a fragment thereof, such as enriching DNA in a sample, may include any suitable enrichment techniques. In some embodiments, enrichment of DNA may include enrichment through molecular inversion probes, in solution capture, pulldown probes, bait sets, standard PCR, multiplex PCR, hybrid capture, endonuclease digestion, DNase I hypersensitivity, and selective circularization. Enrichment can be achieved through negative selection of nucleic acids by eliminating undesired material. This sort of enrichment includes ‘footprinting’ techniques or ‘subtractive’ hybrid capture. During the former, the target sample is safe from nuclease activity through the protection of protein or by single and double stranded arrangements. During the latter, nucleic acids that bind ‘bait’ probes are eliminated.

In some embodiments, enriching can comprise amplification using target-specific primers. In some embodiments, amplification is performed subsequent to another form of enrichment. Typically, however, in embodiments where amplification is used for enrichment, the amplification step occurs after treatment with deaminase, to preserve methylation status of the target DNA. In some such embodiments, amplification can include PCR amplification or genome-wide amplification.

In some embodiments, enrichment can occur after treatment with an ACD. Typically, methods used to identity methylated cytosines result in the loss of DNA complexity due to conversion of unmethylated DNA bases to uracil, resulting in 3-base genome and limits the use of sequences that specifically hybridize to a predetermined sequence. Accordingly, typical methods for identifying methylated cytosines are more difficult to use in methods that include enrichment, such as hybrid-enrichment sequencing and amplicon-based targeted sequencing, after conversion of methylated cytosines. In contrast, because of (i) the 5mC to T conversion by ACDs and (ii) only a small percentage of cytosines are methylated and expected to be converted by an ACD. Examples of enrichment-based methods that can be used after treatment of a target nucleic acid with an ACD include but are not limited to analyte arrays, use of primers for selective amplification, CRISPR-Cas systems, and molecular cytogenic techniques such as FISH. Examples of arrays include, for instance, methylation arrays for interrogation of selected methylation sites across a genome (e.g., the Infinium MethylationEPIC BeadChip, Illumina).

### Attachment of Universal Adapters

In some embodiments, a target nucleic acid used in a method, composition, or kit described herein can include a universal adapter attached to each end. A target nucleic acid having a universal adapter at each end can be referred to as a “modified target nucleic acid.” Methods for attaching a universal adapter to each end of a target nucleic acid used in a method described herein are known to the person skilled in the art. The attachment can be through tagmentation using transposase complexes (WO 2016/130704), or through standard library preparation techniques using ligation (U.S. Pat. Pub. No. 2018/0305753). Attachment of a universal adapter to the ends of a target nucleic acid can occur before or after treatment of the target nucleic acid with an ACD.

In one embodiment, double-stranded target nucleic acids from a sample, e.g., a fragmented sample that has been contacted with an ACD and converted from single-stranded to double-stranded nucleic acids, are treated by first ligating identical universal adaptor molecules to the 5′ and 3′ ends of the double-stranded target nucleic acids. In one embodiment, the universal adapters are “matched” adapters or Y-adapters because the two strands of the adaptors are formed by annealing complementary polynucleotide strands. In one embodiment, the universal adapters used in the method of the disclosure are referred to as “mismatched” adaptors because the adaptors include a region of sequence mismatch, i.e., they are not formed by annealing fully complementary polynucleotide strands. The general features of mismatched adaptors are further described in Gormley et al., U.S. Pat. No. 7,741,463, and Bignell et al., U.S. Pat. No. 8,053,192). The universal adaptor typically includes universal capture binding sequences that aid in immobilizing the target nucleic acids on an array for subsequent sequencing, and universal primer binding sites useful for the sequencing. In another embodiment, double-stranded target nucleic acids from a sample, a sample that has been contacted with an ACD and converted from single-stranded to double-stranded nucleic acids, are subjected to tagmentation with a transposome complex that inserts a universal adapter, or sequences that can be used to add a universal adapter, into a target nucleic acid.

A universal adapter can optionally include at least one index. An index can be used as a marker characteristic of the source of particular target nucleic acids on a flow cell (U.S. Pat. No. 8,053,192). Generally, the index is a synthetic sequence of nucleotides that is part of the universal adapter which is added to the target nucleic acids as part of the library preparation step. Accordingly, an index is a nucleic acid sequence which is attached to each of the target molecules of a particular sample, the presence of which is indicative of, or is used to identify, the sample or source from which the target molecules were isolated.

Preferably an index may be up to 20 nucleotides in length, more preferably 1-10 nucleotides, and most preferably 4-6 nucleotides in length. A four nucleotide index gives a possibility of multiplexing 256 samples on the same array, a six base index enables 4096 samples to be processed on the same array.

The precise nucleotide sequence of the universal adapters is generally not material to the disclosure and may be selected by the user such that the desired sequence elements are ultimately included in the common sequences of the plurality of different modified target nucleic acids, for example, to provide for the universal capture binding sequences for immobilizing the target nucleic acids on an array for subsequent sequencing, and binding sites for particular sets of universal amplification primers and/or sequencing primers. Additional sequence elements may be included, for example, to provide binding sites for sequencing primers which will ultimately be used in sequencing of target nucleic acids in the library, sequencing of an index, or products derived from amplification of the target nucleic acids in the library, for example on a solid support.

In order to prepare a library of deaminase-treated DNA for analysis using a sequencing platform, it may be useful to make additional modifications to the target DNA, either prior to or after treatment with ACD. In some embodiments, single-stranded deaminase-treated DNA is prepared for sequencing using a single-stranded library preparation method, as is known in the art. Such methods include, but are not limited to, template switching based second strand synthesis, adapters containing a single-stranded splint overhang, and the like. Reagents for performing single-stranded library preparation methods are commercially available. Examples include xGen ssDNA & Low-Input DNA Library Prep Kit (Integrated DNA Technologies catalog number 10009859), previously sold as Accel-NGS (Swift Biosciences), NGS Single Stranded DNA Library Prep Kit (BioDynami catalog number 30082). Another example includes single-reaction single-stranded library (SRSLY) as set forth in Troll et al., BMC Genomics 20, 1023 (2019).

In some embodiments, library preparation modifications are made to double-stranded target DNA prior to treatment with an ACD. Methods for library preparation of double-stranded DNA template are known in the art, and include Y-adaptor ligation, transposome-based tagmentation, and the like. It will be appreciated by those of skill in the art that methods of double-strand library preparation often include one or more amplification steps using for example, PCR. In such methods, the amplification step may be deferred until after ACD treatment, to preserve the methylation status of the template strand. For example, in Y-adapter ligation methods, the Y-adapters can be ligated to the double-stranded template, after which the adapter-ligated template DNA is denatured and treated with an ACD as described elsewhere herein. Following treatment with an ACD, the resulting treated single-strand DNA molecules can be amplified using PCR, bridge amplification, and other methods as are commonly known in the art.

### Preparation of Immobilized Samples for Sequencing

The library of modified target nucleic acids, e.g., target nucleic acids having universal adapters at each end, can be prepared for sequencing. Methods for attaching modified target nucleic acids to a substrate are known in the art. In one embodiment, modified fragments are enriched using a plurality of capture oligonucleotides having specificity for the modified fragments, and the capture oligonucleotides can be immobilized on a surface of a solid substrate such as a flow cell or a bead. For instance, capture oligonucleotides can include a first member of a universal binding pair, and where a second member of the binding pair is immobilized on a surface of a solid substrate. Likewise, methods for amplifying immobilized target nucleic acids include, but are not limited to, bridge amplification and exclusion amplification (also referred to as kinetic exclusion amplification (KEA). Methods for immobilizing and amplifying prior to sequencing are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

A pooled sample can be immobilized in preparation for sequencing. Sequencing can be performed as an array of single molecules or can be amplified prior to sequencing. The amplification can be carried out using one or more immobilized primers. The immobilized primer(s) can be, for instance, a lawn on a planar surface, or on a pool of beads. The pool of beads can be isolated into an emulsion with a single bead in each “compartment” of the emulsion. At a concentration of only one template per “compartment,” only a single template is amplified on each bead.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, where one primer is anchored to a bead and the other is in free solution, and colony formation in solid phase gel matrices wherein one primer is anchored to the surface, and one is in free solution.

In some embodiments, the solid support comprises a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. Nos. 8,778,848, 8,778,849 and 9,079,148, and U.S. Pat. Appl. Pub. No. 2014/0243224.

In some embodiments, the solid support includes an array of wells or depressions in a surface. This may be fabricated as is generally known in the art using a variety of techniques, including, but not limited to, photolithography, stamping techniques, molding techniques and micro-etching techniques. As will be appreciated by those of skill in the art, the technique used will depend on the composition and shape of the array substrate.

The features in a patterned surface can be wells in an array of wells (e.g., microwells or nanowells) on glass, silicon, plastic or other suitable solid supports with patterned, covalently-linked gel such as poly(N-(5-azidoacetamidylpentyl) acrylamide-co-acrylamide) (PAZAM, see, for example, US Pub. No. 2013/184796, WO 2016/066586, and WO 2015/002813). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However, in many embodiments the gel need not be covalently linked to the wells. For example, in some conditions silane free acrylamide (SFA, see, for example, U.S. Pat. No. 8,563,477) which is not covalently attached to any part of the structured substrate, can be used as the gel material.

In particular embodiments, a structured substrate can be made by patterning a solid support material with wells (e.g., microwells or nanowells), coating the patterned support with a gel material (e.g., PAZAM, SFA, or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of modified target nucleic acids can then be contacted with the polished substrate such that individual modified target nucleic acids will seed individual wells via interactions with primers attached to the gel material; however, the target nucleic acids will not occupy the interstitial regions due to absence or inactivity of the gel material. Amplification of the modified target nucleic acids will be confined to the wells since absence or inactivity of gel in the interstitial regions prevents outward migration of the growing nucleic acid colony. The process can be conveniently manufactured, being scalable and utilizing conventional micro- or nanofabrication methods.

Although the disclosure encompasses “solid-phase” amplification methods in which only one amplification primer is immobilized (the other primer usually being present in free solution), in one embodiment the solid support is provided with both the forward and the reverse primers immobilized. In practice, there will be a plurality of identical forward primers and/or a plurality of identical reverse primers immobilized on the solid support, since the amplification process requires an excess of primers to sustain amplification. References herein to forward and reverse primers are to be interpreted accordingly as encompassing a plurality of such primers unless the context indicates otherwise.

As will be appreciated by the skilled reader, any given amplification reaction requires at least one type of forward primer and at least one type of reverse primer specific for the template to be amplified. However, in certain embodiments the forward and reverse primers may include template-specific portions of identical sequence, and may have entirely identical nucleotide sequence and structure (including any non-nucleotide modifications). In other words, it is possible to carry out solid-phase amplification using only one type of primer, and such single-primer methods are encompassed within the scope of the disclosure. Other embodiments may use forward and reverse primers which contain identical template-specific sequences but which differ in some other structural features. For example, one type of primer may contain a non-nucleotide modification which is not present in the other.

Primers for solid-phase amplification are preferably immobilized by single point covalent attachment to the solid support at or near the 5′ end of the primer, leaving the template-specific portion of the primer free to anneal to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular embodiment, the primer may include a sulphur-containing nucleophile, such as phosphorothioate or thiophosphate, at the 5′ end. In the case of solid-supported polyacrylamide hydrogels, this nucleophile will bind to a bromoacetamide group present in the hydrogel. A more particular means of attaching primers and templates to a solid support is via 5′ phosphorothioate attachment to a hydrogel comprised of polymerized acrylamide and N-(5-bromoacetamidylpentyl) acrylamide (BRAPA), as described in Int. Pub. No. WO 05/065814.

Certain embodiments of the disclosure may make use of solid supports that include an inert substrate or matrix (e.g., glass slides, polymer beads, etc.) which has been “functionalized,” for example by application of a layer or coating of an intermediate material including reactive groups which permit covalent attachment to biomolecules, such as polynucleotides. Examples of such supports include, but are not limited to, polyacrylamide hydrogels supported on an inert substrate such as glass. In such embodiments, the biomolecules (e.g., polynucleotides) may be directly covalently attached to the intermediate material (e.g., the hydrogel), but the intermediate material may itself be non-covalently attached to the substrate or matrix (e.g., the glass substrate). The term “covalent attachment to a solid support” is to be interpreted accordingly as encompassing this type of arrangement.

The pooled samples may be amplified on beads wherein each bead contains a forward and reverse amplification primer. In one embodiment, a library of modified target nucleic acids is used to prepare clustered arrays of nucleic acid colonies, analogous to those described in U.S. Pub. No. 2005/0100900, U.S. Pat. No. 7,115,400, WO 00/18957 and WO 98/44151 by solid-phase amplification and more particularly solid phase isothermal amplification. The terms “cluster” and “colony” are used interchangeably herein to refer to a discrete site on a solid support including a plurality of identical immobilized nucleic acid strands and a plurality of identical immobilized complementary nucleic acid strands. The term “clustered array” refers to an array formed from such clusters or colonies. In this context, the term “array” is not to be understood as requiring an ordered arrangement of clusters.

The term “solid phase” or “surface” is used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

Clustered arrays can be prepared using either a process of thermocycling, as described in WO 98/44151, or a process whereby the temperature is maintained as a constant, and the cycles of extension and denaturing are performed using changes of reagents. Such isothermal amplification methods are described in patent application numbers WO 02/46456 and U.S. Pub. No. 2008/0009420.

It will be appreciated that any of the amplification methodologies described herein or generally known in the art may be used with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354. The above amplification methods may be employed to amplify one or more nucleic acids of interest. For example, PCR, including multiplex PCR, SDA, TMA, NASBA and the like may be utilized to amplify immobilized DNA fragments. In some embodiments, primers directed specifically to the polynucleotide of interest are included in the amplification reaction.

Other suitable methods for amplification of polynucleotides may include oligonucleotide extension and ligation, rolling circle amplification (RCA) (Lizardi et al., Nat. Genet. 19:225-232 (1998)) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835) technologies. It will be appreciated that these amplification methodologies may be designed to amplify immobilized DNA fragments. For example, in some embodiments, the amplification method may include ligation probe amplification or oligonucleotide ligation assay (OLA) reactions that contain primers directed specifically to the nucleic acid of interest. In some embodiments, the amplification method may include a primer extension-ligation reaction that contains primers directed specifically to the nucleic acid of interest. As a non-limiting example of primer extension and ligation primers that may be specifically designed to amplify a nucleic acid of interest, the amplification may include primers used for the GoldenGate assay (Illumina, Inc., San Diego, CA) as exemplified by U.S. Pat. Nos. 7,582,420 and 7,611,869.

DNA nanoballs can also be used in combination with methods, systems, compositions and kits as described herein. Methods for creating and using DNA nanoballs for genomic sequencing can be found at, for example, US patents and publications U.S. Pat. No. 7,910,354, 2009/0264299, 2009/0011943, 2009/0005252, 2009/0155781, 2009/0118488 and as described in, for example, Drmanac et al. (2010, Science 327 (5961): 78-81). Briefly, following production of modified target nucleic acids, the modified target nucleic acids are circularized and amplified by rolling circle amplification (Lizardi et al., 1998. Nat. Genet. 19:225-232; US 2007/0099208 A1). The extended concatemeric structure of the amplicons promotes coiling creates compact DNA nanoballs. The DNA nanoballs can be captured on substrates, preferably to create an ordered or patterned array such that distance between each nanoball is maintained thereby allowing sequencing of the separate DNA nanoballs. In some embodiments such as those used by Complete Genomics (Mountain View, Calif.), consecutive rounds of adapter addition, amplification, and digestion are carried out prior to circularization to produce head to tail constructs having several target nucleic acids separated by adapter sequences.

Exemplary isothermal amplification methods that may be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587. Other non-PCR-based methods that may be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyper-branched strand displacement amplification which is described in, for example Lage et al., Genome Res. 13:294-307 (2003). Isothermal amplification methods may be used with, for instance, the strand-displacing Phi 29 polymerase or Bst DNA polymerase large fragment, 5′→3′ exo—for random primer amplification of genomic DNA. The use of these polymerases takes advantage of their high processivity and strand displacing activity. High processivity allows the polymerases to produce fragments that are 10-20 kb in length. As set forth above, smaller fragments may be produced under isothermal conditions using polymerases having low processivity and strand-displacing activity such as Klenow polymerase. Additional description of amplification reactions, conditions and components are set forth in detail in the disclosure of U.S. Pat. No. 7,670,810.

In some embodiments, isothermal amplification can be performed using kinetic exclusion amplification (KEA), also referred to as exclusion amplification (ExAmp). A nucleic acid library of the present disclosure can be made using a method that includes a step of reacting an amplification reagent to produce a plurality of amplification sites that each includes a substantially clonal population of amplicons from an individual target nucleic acid that has seeded the site. In some embodiments, the amplification reaction proceeds until a sufficient number of amplicons are generated to fill the capacity of the respective amplification site. Filling an already seeded site to capacity in this way inhibits target nucleic acids from landing and amplifying at the site thereby producing a clonal population of amplicons at the site. In some embodiments, apparent clonality can be achieved even if an amplification site is not filled to capacity prior to a second target nucleic acid arriving at the site. Under some conditions, amplification of a first target nucleic acid can proceed to a point that a sufficient number of copies are made to effectively outcompete or overwhelm production of copies from a second target nucleic acid that is transported to the site. For example, in an embodiment that uses a bridge amplification process on a circular feature that is smaller than 500 nm in diameter, it has been determined that after 14 cycles of exponential amplification for a first target nucleic acid, contamination from a second target nucleic acid at the same site will produce an insufficient number of contaminating amplicons to adversely impact sequencing-by-synthesis analysis on an Illumina sequencing platform.

In some embodiments, amplification sites in an array can be, but need not be, entirely clonal. Rather, for some applications, an individual amplification site can be predominantly populated with amplicons from a first modified target nucleic acid and can also have a low level of contaminating amplicons from a second modified target nucleic acid. An array can have one or more amplification sites that have a low level of contaminating amplicons so long as the level of contamination does not have an unacceptable impact on a subsequent use of the array. For example, when the array is to be used in a detection application, an acceptable level of contamination would be a level that does not impact signal to noise or resolution of the detection technique in an unacceptable way. Accordingly, apparent clonality will generally be relevant to a particular use or application of an array made by the methods set forth herein. Exemplary levels of contamination that can be acceptable at an individual amplification site for particular applications include, but are not limited to, at most 0.1%, 0.5%, 1%, 5%, 10% or 25% contaminating amplicons. An array can include one or more amplification sites having these exemplary levels of contaminating amplicons. For example, up to 5%, 10%, 25%, 50%, 75%, or even 100% of the amplification sites in an array can have some contaminating amplicons. It will be understood that in an array or other collection of sites, at least 50%, 75%, 80%, 85%, 90%, 95% or 99% or more of the sites can be clonal or apparently clonal.

In some embodiments, kinetic exclusion can occur when a process occurs at a sufficiently rapid rate to effectively exclude another event or process from occurring. Take for example the making of a nucleic acid array where sites of the array are randomly seeded with modified target nucleic acids from a solution and copies of the modified target nucleic acids are generated in an amplification process to fill each of the seeded sites to capacity. In accordance with the kinetic exclusion methods of the present disclosure, the seeding and amplification processes can proceed simultaneously under conditions where the amplification rate exceeds the seeding rate. As such, the relatively rapid rate at which copies are made at a site that has been seeded by a first target nucleic acid will effectively exclude a second nucleic acid from seeding the site for amplification. Kinetic exclusion amplification methods can be performed as described in detail in the disclosure of U.S. Pat. Appl. Pub. No. 2013/0338042.

Kinetic exclusion can exploit a relatively slow rate for initiating amplification (e.g., a slow rate of making a first copy of a modified target nucleic acids) vs. a relatively rapid rate for making subsequent copies of the modified target nucleic acids (or of the first copy of the modified target nucleic acids). In the example of the previous paragraph, kinetic exclusion occurs due to the relatively slow rate of modified target nucleic acids seeding (e.g., relatively slow diffusion or transport) vs. the relatively rapid rate at which amplification occurs to fill the site with copies of the modified target nucleic acid seed. In another exemplary embodiment, kinetic exclusion can occur due to a delay in the formation of a first copy of a modified target nucleic acid that has seeded a site (e.g., delayed or slow activation) vs. the relatively rapid rate at which subsequent copies are made to fill the site. In this example, an individual site may have been seeded with several different modified target nucleic acids (e.g., several modified target nucleic acids can be present at each site prior to amplification). However, first copy formation for any given modified target nucleic acid can be activated randomly such that the average rate of first copy formation is relatively slow compared to the rate at which subsequent copies are generated. In this case, although an individual site may have been seeded with several different modified target nucleic acids, kinetic exclusion will allow only one of those to be amplified. More specifically, once a first modified target nucleic acid has been activated for amplification, the site will rapidly fill to capacity with its copies, thereby preventing copies of a second modified target nucleic acid from being made at the site.

In one embodiment, the method is carried out to simultaneously (i) transport modified target nucleic acids to amplification sites at an average transport rate, and (ii) amplify the modified target nucleic acids that are at the amplification sites at an average amplification rate, wherein the average amplification rate exceeds the average transport rate (U.S. Pat. No. 9,169,513). Accordingly, kinetic exclusion can be achieved in such embodiments by using a relatively slow rate of transport. For example, a sufficiently low concentration of modified target nucleic acids can be selected to achieve a desired average transport rate, lower concentrations resulting in slower average rates of transport. Alternatively or additionally, a high viscosity solution and/or presence of molecular crowding reagents in the solution can be used to reduce transport rates. Examples of useful molecular crowding reagents include, but are not limited to, polyethylene glycol (PEG), ficoll, dextran, or polyvinyl alcohol. Exemplary molecular crowding reagents and formulations are set forth in U.S. Pat. No. 7,399,590. Another factor that can be adjusted to achieve a desired transport rate is the average size of the target nucleic acids.

An amplification reagent can include further components that facilitate amplicon formation, and in some cases increase the rate of amplicon formation. An example is a recombinase. Recombinase can facilitate amplicon formation by allowing repeated invasion/extension. More specifically, recombinase can facilitate invasion of a modified target nucleic acid by the polymerase and extension of a primer by the polymerase using the modified target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g., via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single-stranded binding (SSB) protein is particularly useful as SSB can further facilitate amplification. Exemplary formulations for recombinase-facilitated amplification include those sold commercially as TwistAmp kits by TwistDx (Cambridge, UK). Useful components of recombinase-facilitated amplification reagent and reaction conditions are set forth in U.S. Pat. Nos. 5,223,414 and 7,399,590.

Another example of a component that can be included in an amplification reagent to facilitate amplicon formation and in some cases to increase the rate of amplicon formation is a helicase. Helicase can facilitate amplicon formation by allowing a chain reaction of amplicon formation. The process can occur more rapidly than standard PCR since a denaturation cycle (e.g., via heating or chemical denaturation) is not required. As such, helicase-facilitated amplification can be carried out isothermally. A mixture of helicase and single-stranded binding (SSB) protein is particularly useful as SSB can further facilitate amplification. Exemplary formulations for helicase-facilitated amplification include those sold commercially as IsoAmp kits from Biohelix (Beverly, MA). Further, examples of useful formulations that include a helicase protein are described in U.S. Pat. Nos. 7,399,590 and 7,829,284.

Yet another example of a component that can be included in an amplification reagent to facilitate amplicon formation and in some cases increase the rate of amplicon formation is an origin binding protein.

### Methods of Sequencing

Following attachment of modified target nucleic acids to a surface, the sequence of the immobilized and amplified modified target nucleic acids is determined. Sequencing can be carried out using any suitable sequencing technique, and methods for determining the sequence of immobilized and amplified modified target nucleic acids, including strand re-synthesis, are known in the art and are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a modified target nucleic acid can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

In one embodiment, a nucleotide monomer includes locked nucleic acids (LNAs) or bridged nucleic acids (BNAs). The use of LNAs or BNAs in a nucleotide monomer increases hybridization strength between a nucleotide monomer and a sequencing primer sequence present on an immobilized modified modified target nucleic acid.

SBS can use nucleotide monomers that have a terminator moiety or those that lack any terminator moieties. Methods using nucleotide monomers lacking terminators include, for example, pyrosequencing and sequencing using y-phosphate-labeled nucleotides, as set forth in further detail herein. In methods using nucleotide monomers lacking terminators, the number of nucleotides added in each cycle is generally variable and dependent upon the template sequence and the mode of nucleotide delivery. For SBS techniques that use nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can use nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments where two or more different nucleotides are present in a sequencing reagent, the different nucleotides can be distinguishable from each other, or alternatively the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026. This approach is being commercialized by Solexa (now Illumina Inc.), and is also described in WO 91/06678 and WO 07/123,744. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In some reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth herein.

In particular embodiments some or all of the nucleotide monomers can include reversible terminators. In such embodiments, reversible terminators/cleavable fluorophores can include fluorophores linked to the ribose moiety via a 3′ ester linkage (Metzker, Genome Res. 15:1767-1776 (2005)). Other approaches have separated the terminator chemistry from the cleavage of the fluorescence label (Ruparel et al., Proc Natl Acad Sci USA 102:5932-7 (2005)). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673, and 7,057,026.

Additional exemplary SBS systems and methods which can be used with the methods and systems described herein are described in U.S. Pub. Nos. 2007/0166705, 2006/0188901, 2006/0240439, 2006/0281109, 2012/0270305, and 2013/0260372, U.S. Pat. No. 7,057,026, PCT Publication No. WO 05/065814, U.S. Patent Application Publication No. 2005/0100900, and PCT Publication Nos. WO 06/064199 and WO 07/010,251.

Some embodiments can use detection of four different nucleotides using fewer than four different labels. For example, SBS can be performed using methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected. In no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g., dATP having a label that is detected in the first channel when excited by a first excitation wavelength), a second nucleotide type that is detected in a second channel (e.g., dCTP having a label that is detected in the second channel when excited by a second excitation wavelength), a third nucleotide type that is detected in both the first and the second channel (e.g., dTTP having at least one label that is detected in both channels when excited by the first and/or second excitation wavelength) and a fourth nucleotide type that lacks a label that is not, or minimally, detected in either channel (e.g., dGTP having no label).

Further, as described in U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can use sequencing by ligation techniques. Such techniques use DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597.

Some embodiments can use nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003)). In such embodiments, the modified target nucleic acid passes through a nanopore. The nanopore can be a synthetic pore or biological membrane protein, such as a-hemolysin. As the modified target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008)). Data obtained from nanopore sequencing can be stored, processed and analyzed as set forth herein. In particular, the data can be treated as an image in accordance with the exemplary treatment of optical images and other images that is set forth herein.

Some embodiments can use methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414, or nucleotide incorporations can be detected with zero-mode waveguides as described, for example, in U.S. Pat. No. 7,315,019, and using fluorescent nucleotide analogs and engineered polymerases as described, for example, in U.S. Pat. No. 7,405,281 and U.S. Pub. No. 2008/0108082. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008)). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pub. Nos. 2009/0026082; 2009/0127589; 2010/0137143; and 2010/0282617. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different modified target nucleic acids are manipulated simultaneously. In particular embodiments, different modified target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the modified target nucleic acids can be in an array format. In an array format, the modified target nucleic acids can be typically bound to a surface in a spatially distinguishable manner. The modified target nucleic acids can be bound by direct covalent attachment, attachment to a bead or other particle or binding to a polymerase or other molecule that is attached to the surface. The array can include a single copy of a modified target nucleic acid at each site (also referred to as a feature) or multiple copies having the same sequence can be present at each site or feature. Multiple copies can be produced by amplification methods such as, bridge amplification or emulsion PCR as described in further detail herein.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of cm², in parallel. Accordingly, the present disclosure provides integrated systems capable of preparing and detecting nucleic acids using techniques known in the art such as those exemplified herein. Thus, an integrated system of the present disclosure can include fluidic components capable of delivering amplification reagents and/or sequencing reagents to one or more immobilized modified target nucleic acids, the system including components such as pumps, valves, reservoirs, fluidic lines and the like. A flow cell can be configured and/or used in an integrated system for detection of target nucleic acids. Exemplary flow cells are described, for example, in U.S. Pat. Nos. 8,241,573 and 8,951,781. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Pat. No. 8,951,781.

While the embodiments presented herein are generally described using a sequencing platform (such as a sequencing by synthesis platform) as a readout, one of ordinary skill in the art will recognize that nucleic acids modified by the ACDs presented herein can also be detected using any other suitable readout methodology. For example, the location and identity of modified cytosines can be assessed using a microarray. Any of a variety of analyte arrays (also referred to as “microarrays”) known in the art can be used in a method or system set forth herein. A typical array contains analytes, each having an individual probe or a population of probes. In the latter case, the population of probes at each analyte is typically homogenous having a single species of probe. For example, in the case of a nucleic acid array, each analyte can have multiple nucleic acid molecules each having a common sequence. However, in some implementations the populations at each analyte of an array can be heterogeneous. Similarly, protein arrays can have analytes with a single protein or a population of proteins typically, but not always, having the same amino acid sequence. The probes can be attached to the surface of an array for example, via covalent linkage of the probes to the surface or via non-covalent interaction(s) of the probes with the surface. In some implementations, probes, such as nucleic acid molecules, can be attached to a surface via a gel layer as described, for example, in U.S. patent application Ser. No. 13/784,368 and US Pat. App. Pub. No. 2011/0059865 A1.

Example arrays include, without limitation, a BeadChip Array available from Illumina, Inc. (San Diego, Calif.) or others such as those where probes are attached to beads that are present on a surface (e.g., beads in wells on a surface) such as those described in U.S. Pat. Nos. 6,266,459; 6,355,431; 6,770,441; 6,859,570; or 7,622,294; or PCT Publication No. WO 00/63437. Further examples of commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies. A spotted microarray can also be used in a method or system according to some implementations of the present disclosure. An example spotted microarray is a CodeLink™ Array available from Amersham Biosciences. Another microarray that is useful is one that is manufactured using inkjet printing methods such as SurePrint™ Technology available from Agilent Technologies.

In a specific embodiment, an ACD as presented herein can be used to convert 5-methyl cytosine (5mC) to thymidine (T) by deamination as described herein, such as by providing a sample of DNA suspected of including single-stranded DNA including at least one 5-methyl cytosine (5mC), at least one 5-hydroxymethyl cytosine (5hmC), at least one 5-formyl cytosine (5fC), at least one 5-carboxy cytosine (5CaC), or a combination thereof; contacting the DNA with the ACD under conditions suitable for conversion of 5-methylcytosine (5mC) to thymidine (T) by deamination at a greater rate than conversion of cytosine (C) to uracil (U) by deamination, to result in converted single-stranded DNA, wherein 5mC, 5hmC, 5fC, and/or 5CaC are converted to T.

In a specific embodiment, an ACD of the present disclosure can be used to detect 5hmC as described herein, such as by providing a sample of DNA suspected of including single-stranded DNA that has at least one 5-hydroxymethyl cytosine (5hmC); contacting the DNA with the ACD under conditions suitable for conversion of cytosine to uracil and 5mC to thymidine and no detectable conversion of 5hmC to 5hmU.

The converted single-stranded DNA can then be processed as needed to facilitate hybridization to a microarray. For example, the converted DNA can be amplified. Any one of a number of amplification methods as are known in the art can be performed. For example, whole-genome amplification or amplification using universal primers that hybridize to a common region in the converted DNA, such as an adaptor sequence, can be used. Additionally or alternatively, the converted DNA can be fragmented. Fragmentation can be performed prior to or following amplification, or in the absence of amplification. Any one of a number of fragmentation methods as are known in the art can be performed. As one example, fragmentation can be performed using an enzymatic process, such as a restriction endonuclease or other enzyme capable of cleaving the converted DNA. As another example, fragmentation can be performed using mechanical means, such as shearing using, for example a sonication device such as those supplied by Covaris. The fragmented converted DNA can then be precipitated and/or resuspended in a buffer suitable for hybridization to a microarray. Following hybridization, the methylation state of regions of interest, such as a specific CpG locus or loci, can be interrogated at specific locations on the microarray. Methods of preparing converted DNA for microarray analysis are known in the art. One example of such methods is described in the Methylation Protocol Guide for the Infinium HD Assay from Illumina (San Diego, CA). Whereas such a protocol guide may describe use of a microarray designed for interrogation of bisulfite-converted DNA, it will be understood that array features, specifically probe sequences, can be specifically designed for DNA that has not been bisulfite converted. As an example, a commercially available microarray such as the Infinium MethylationEPIC BeadChip (Illumina) is specifically designed to hybridize with DNA fragments with reduced complexity, as found in bisulfite converted DNA, where most if not all cytosines are converted to thymidine. Thus, for example, the same CpG sites can be interrogated in non-bisulfite-converted DNA by using a microarray including probes designed to hybridize to the same regions of native, non-bisulfite-converted DNA. One of skill in the art could readily obtain such a microarray. In one embodiment, a custom array could be designed using the manifest for an array such as the Infinium MethylationEPIC BeadChip, by using the “Forward Sequence” to identify a probe sequence including native DNA sequence that covers a similar or identical sequence region for the allele-specific probe sequences, which are designed to hybridize to DNA sequences where most or all cytosines have been converted to thymidine. Using such an array designed to hybridize to native (non-bisulfite-converted) DNA sequences, the methodologies and analysis methods described in the Methylation Protocol Guide for the Infinium HD Assay from Illumina (San Diego, CA) could be followed to identify methylated CpG sites in the sample DNA.

### Compositions

The present disclosure also provides compositions that include an ACD described herein. The composition can include one or more additional other components in addition to the ACD. For example, the other component can include a single-stranded DNA or RNA substrate that includes, or is suspected of including, at least one modified cytosine, such as a 5-methyl cytosine, a 5-hydroxymethyl cytosine, or a combination thereof. In another example, a single-stranded DNA or RNA substrate can be one including one or more known modified cytosine, e.g., a single-stranded DNA or RNA substrate that can be used as a control to measure conversion efficiency. In another example, the other component can include a buffer, such as a buffer described herein, for example a citrate buffer, a sodium acetate buffer, or a Bis-Tris buffer, or HEPES. In another example, the other component can include a reductant, including but not limited to, DTT and/or TCEP, as well as Zn, or a denaturant, such as DMSO or betaine.

A composition can include a polynucleotide encoding an ACD described herein. The polynucleotide can be present in a vector, such as a plasmid or virus vector. A vector that includes the polynucleotide can be present in a host cell, such as E. coli.

### Kits

The present disclosure also provides kits for determining the methylation status of DNA or RNA. A kit includes at least one ACD described herein and one or more other components in a suitable packaging material in an amount sufficient for at least one reaction. Examples of other components include a positive control polynucleotide, such as a single-stranded DNA including one or more known modified cytosines for use in measuring conversion efficiency, or a negative control polynucleotide, such as a single-stranded DNA including cytosines. Another component can be a glucosyltransferase, such as T4-beta glucosyltransferase. Optionally, other reagents such as buffers and solutions needed to use the ACD and nucleotide solution are also included. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the components can be used for determining the methylation status of DNA or RNA. In addition, the packaging material contains instructions indicating how the materials within the kit are employed to practice a reaction with an ACD. As used herein, the term “package” refers to a solid matrix or material such as glass, plastic, paper, foil, and the like, capable of holding within fixed limits the components of the kit. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1. An altered cytidine deaminase (ACD) having C-selective deaminase activity and comprising one or more stability-enhancing alteration, preferably two or more stability-enhancing alterations, wherein conversion of cytosine (C) to uracil (U) by deamination occurs at a greater rate than conversion of 5-methyl cytosine (5mC) to thymidine (T) by deamination.
Aspect 2. The ACD of any aspect herein, wherein the C-selective deaminase activity of the ACD is the result of one or more substitution mutations, deletions, or insertions.
Aspect 3. The ACD of any aspect herein, wherein C-selective deaminase comprises (a) a substitution mutation at position functionally equivalent to Y130, optionally wherein the substitution is preferably L, F or I, preferably L; (b) a substitution mutation at position functionally equivalent to Y132, optionally wherein the substitution is preferably H or P; (c) a substitution mutation at a position functionally equivalent to D133, wherein the substitution is any amino acid except P or Y; or (d) a combination of (a), (b) and (c).
Aspect 4. The ACD of any aspect herein, wherein (a) the substitution at D133 is W, D, K or R.
Aspect 5. The ACD of any aspect herein, wherein (d) comprises a combination of (a) and (b); (a) and (c); (b) and (c); or (a), (b), and (c).
Aspect 6. The ACD of any aspect herein, wherein the one or more amino acid substitution mutation comprise a substitution at a position functionally equivalent to Tyr130 in a wild-type ACD, at a position functionally equivalent to Tyr132 in a wild-type ACD, or a combination thereof, wherein the wild-type ACD is a wild-type APOBEC3A (SEQ ID NO:3).
Aspect 7. The ACD of any aspect herein, wherein the at least one amino acid substitution mutation at the position functionally equivalent to Tyr130 is leucine (L), phenylalanine (F), or isoleucine (I).
Aspect 8. The ACD of any aspect herein, wherein the at least one amino acid substitution mutation at the position functionally equivalent to Tyr132 is histidine or proline.
Aspect 9. The ACD of any aspect herein, wherein the ACD comprises the substitution mutations Tyr130Leu/Tyr132His, Tyr130Phe/Tyr132Pro, Tyr132Pro, Tyr130Leu/Tyr132His/D133X, Tyr130Phe/Tyr132Pro/D133X, or Tyr132Pro/D133X, or Tyr130Ile/Tyr132Pro/D133X, wherein X is W, D, K, or R.
Aspect 10. The ACD of any aspect herein, wherein the ACD comprises SEQ ID NO: 80, SEQ ID NO:81, or SEQ ID NO:82, or a sequence having at least 75% or more sequence identity to SEQ ID NO:80, SEQ ID NO:81, or SEQ ID NO:82, preferably at least 90%.
Aspect 11. The ACD of any aspect herein, comprising one or more alterations that enhance the selectivity of an ACD for C comprising (i) a substitution mutation at a position functionally equivalent to S103X, E138X, Q115X, R28X, Y136X, C108 X, or A112X, X is an amino acid substitution different from the wild-type amino acid at that position, (ii) a deletion of amino acids at positions functionally equivalent to ΔI26-G27, where the position number designation is functionally equivalent to the position in a wild-type APOBEC3A, (iii) an insertion at positions functionally equivalent to Σ130-131:X, Σ132-133:X, Σ130-131:X, Σ131-132:X, Σ131-132:X, Σ102-103:X, Σ138-139:X, Σ137-138:X, or Σ134-135:X, where the position number designation is functionally equivalent to the position in a wild-type APOBEC3A (SEQ ID NO: 3); or (iv) a combination of (i) and (ii), (ii) and (iii), (i) and (ii), or (i), (ii), and (iii).
Aspect 12. The ACD of any aspect herein, wherein the ACD comprises one or more alterations listed in Table 2 or 3.
Aspect 13. The ACD of any aspect herein, wherein the ACD comprises two or more alterations listed in Table 2 or 3.
Aspect 14. The ACD of any aspect herein, wherein the ACD comprises three or more alterations listed in Table 2 or 3, preferably four or more mutations, wherein the ACD has C-selective deaminase activity.
Aspect 15. The ACD of any aspect herein, wherein the C-selective deaminase activity of the ACD is the result of one or more substitution mutations.
Aspect 16. The ACD of any aspect herein, wherein the ACD further comprises at least one stability-enhancing alteration, wherein the stability-enhancing alteration is a substitution mutation, a deletion, an insertion, or a combination thereof.
Aspect 17. The ACD of any aspect herein, wherein the one or more stability-enhancing alterations are selected from (a) substitution mutations at a position functionally equivalent to (a) A112X, A126X, A139X, A148X, A185X, A192X, A59X, A87X, C106X, C161X, C171X, C34X, D145X, D156X, D163X, D167X, D177X, D180X, D41X, D77X, D85X, E109X, E116X, E138X, E157X, E38X, G105X, G108X, G188X, G25X, G27X, H119X, H11X, H16X, H182X, H29X, H51LX, I17X, I26X, I89X, K47X, K60X, L135X, L62X, L78X, M14X, M48X, N117X, N196X, N21X, N42X, P80X, Q115X, Q141X, Q169X, Q184X, R111X, R123X, R189X, R39X, R74X, R91X, S103X, S183X, S187X, S20X, S45X, T118X, T164X, T19X, T31X, T93X, V110X, V79X, L12X, D14X, T19X, G27X, R28X, E38X, F54X, H56X, N57X, Y67X, L73X, D77X, S81X, Y90X, I96X, S97X, C101X, F102X, W104X, C106X, A107X, L114X, V120X, L122X, R128X, Y136X, M142X, A146X, K159X, C161X, L186X, (b) a deletion selected from ΔM1-L12, ΔE36-G53, ΔN61-G68, ΔW104-G105, ΔQ195-N199, or ΔI26-G27, and (c) combinations thereof, wherein the position number designation is functionally equivalent to the position in a wild-type APOBEC3A (SEQ ID NO:3) and X is an amino acid substitution different from the wild-type amino acid at that position.
Aspect 18. The ACD of any aspect herein, wherein the ACD comprises: a. three or more stability-enhancing alterations; b. four or more stability-enhancing alterations; c. five or more stability-enhancing alterations; d. six or more stability-enhancing alterations; e. seven or more stability-enhancing alterations; f. eight or more stability-enhancing alterations; g. nine or more stability-enhancing alterations; h. ten or more stability-enhancing alterations, i. eleven or more stability-enhancing alterations, or k. twelve or more stability-enhancing alterations.
Aspect 19. The ACD of any aspect herein, wherein the ACD comprises 1. thirteen or more stability-enhancing alterations; or m. fourteen or more stability-enhancing alterations.
Aspect 20. The ACD of any one of aspects 16-19, wherein the one or more stability enhancing alterations comprises a substitution mutation at a position functionally equivalent to a stability-enhancing mutation selected from Table 3.
Aspect 21. The ACD of any aspect herein, wherein the one or more stability-enhancing alterations comprises a deletion selected from Table 6.
Aspect 22. The ACD of any aspect herein, wherein the one or more stability-enhancing alterations comprises at least one substitution and at least one deletion, wherein the substitution mutation is at a position functionally equivalent to a stability-enhancing mutation selected from Table 3, and the deletion is a deletion selected from Table 6.
Aspect 23. The ACD of any aspect herein, wherein the one or more alterations comprises a deletion selected from Table 6 and one or more ancillary substitution mutations.
Aspect 24. The ACD of any aspect herein, wherein the deletion and one or more ancillary substitution mutations of the ACD is ΔN61-G68/A59P/K60R, ΔN61-G68/A59L/R69Y, ΔN61-G68/A59L/K60E/R69N, ΔN61-G68/A59P/K60E/R69H, ΔN61-G68/A59P/K60Q/R69H, ΔN61-G68/A59L/R69N, ΔN61-G68/R69D, ΔN61-G68/A59L/K60R, ΔN61-G68/A59P/K60G/R69L, ΔW104-G105/F102R, ΔW104-G105/S103N, ΔW104-G105/F102R/S103N, ΔW104-G105/41-12, ΔW104-G105/Δ1-12/F102R, ΔW104-G105/A1-12/S103N or ΔW104-G105/Δ1-12/F102R/S103N.
Aspect 25. The ACD of any aspect herein, wherein the ACD comprises a combination of stability-enhancing alterations selected from Table 7 or 8.
Aspect 26. The ACD of any aspect herein, wherein the ACD is based on a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3H subfamily, or the APOBEC4 subfamily.
Aspect 27. The ACD of any aspect herein, wherein the ACD is APOBECC3A (SEQ ID NO:3).
Aspect 28. The ACD of any aspect herein, wherein the ACD comprises a ZDD motif H-[P/A/V]-E-X_{[23-28]}-P-C-X_{[2-4]}-C (SEQ ID NO:12).
Aspect 29. The ACD of any aspect herein, wherein the ACD is derived from a member of the APOBEC3A subfamily and comprises X_{[16-26]}-GRXXTXLCYXV-X₁₅-GXXXN-X₁₂-HAEXXF-X₁₄-YXXTWXXSWSPC-X_{[2-4]}-CA-X₅-FL-X₇-LXIXXXR(L/I)Y-X₈-GLXXLXXXG-X₅-M-X₄-FXXCWXXFV-X₆-FXPW-X₁₃-LXXI-X_{[2-6]} (SEQ ID NO:14).
Aspect 30. The ACD of any aspect herein, wherein the at least one selectivity-enhancing alteration is a substitution mutation at a position functionally equivalent to D133, a substitution mutation at a position functionally equivalent to S103X, E138X, Q115X, R28X, Y136X, C108 X, or A112X, a deletion of amino acids at positions functionally equivalent to ΔI26-G27, wherein the position number designation is functionally equivalent to the position in a wild-type APOBEC3A (SEQ ID NO:3) and X is an amino acid substitution different from the wild-type amino acid at that position, or an insertion at positions functionally equivalent to Σ130-131:X, Σ132-133:X, Σ130-131:X, Σ131-132:X, Σ131-132:X, Σ102-103:X, Σ138-139:X, Σ137-138:X, or ≥134-135:X, where the position number designation is functionally equivalent to the position in a wild-type APOBEC3A (SEQ ID NO:3) and X is the amino acid inserted between the two identified amino acids, or a substitution mutation, an insertion, or a deletion selected from Table 2.
Aspect 31. A polynucleotide encoding the ACD of any aspect herein.
Aspect 32. A composition comprising the ACD of any aspect herein and a buffer.
Aspect 33. The composition of any aspect herein, wherein the composition further comprises a sample comprising DNA comprising at least one modified cytosine, wherein the modified cytosine is 5-methyl cytosine (5mC), 5-hydroxymethyl cytosine (5hmC), or a combination thereof.
Aspect 34. The composition of any aspect herein, wherein the DNA comprises single-stranded DNA.
Aspect 35. The composition of any aspect herein, wherein the sample comprises genomic DNA or cell free DNA.
Aspect 36. The composition of any aspect herein, wherein the genomic DNA is from a single cell or is a mixture from a plurality of cells.
Aspect 37. A method comprising: providing a sample of DNA suspected of comprising single-stranded DNA comprising at least one 5-methyl cytosine (5mC), at least one 5-hydroxymethyl cytosine (5hmC), or a combination thereof; contacting the single-stranded DNA with an ACD under conditions suitable for (i) conversion of cytosine (C) to uracil (U) by deamination at a greater rate than 5-methylcytosine (5mC) to thymidine (T), to result in converted single-stranded DNA, wherein the altered cytidine deaminase is the ACD of any aspect herein; and processing the converted single-stranded DNA to produce a sequencing library.
Aspect 38. The method of any aspect herein, wherein the method further comprises, before the providing, denaturing double-stranded DNA present in the sample to result in single-stranded DNA.
Aspect 39. A method comprising: providing a sample of DNA suspected of comprising double-stranded DNA comprising at least one cytosine, 5-methyl cytosine (5mC), at least one 5-hydroxymethyl cytosine (5hmC), or a combination thereof; processing the double-stranded DNA to produce a sequencing library; denaturing the sequencing library to result in a single-stranded DNA; contacting the single-stranded DNA with an ACD under conditions suitable for conversion of C to U by deamination and 5mC to T by deamination at a greater rate than conversion of 5-hydroxymethyl cytosine (5hmC) to 5-hydroxymethyl uracil (5hmU) by deamination, to result in converted single-stranded DNA, wherein the altered cytidine deaminase is the ACD of any aspect herein; and converting the converted single-stranded DNA to a converted double-stranded DNA sequencing library.
Aspect 40. The method of any aspect herein, wherein the processing comprises fragmentation or tagmentation of the double-stranded DNA and addition of a universal sequence to the double-stranded DNA fragments.
Aspect 41. The method of any aspect herein, wherein the universal sequence is part of an adapter added to the double-stranded DNA fragments.
Aspect 42. The method of any aspect herein, wherein the converting comprises amplifying the converted single-stranded DNA to be the converted double-stranded DNA.
Aspect 43. The method of any aspect herein, wherein the sample is a biological sample.
Aspect 44. The method of any aspect herein, wherein the biological sample comprises cell-free DNA.
Aspect 45. The method of any aspect herein, wherein the biological sample comprises a fluid selected from blood or serum.
Aspect 46. The method of any aspect herein, wherein the sample comprises single cells or isolated nuclei.
Aspect 47. The method of any aspect herein, wherein the biological sample comprises a tissue.
Aspect 48. The method of any aspect herein, wherein the tissue comprises tumor tissue.
Aspect 49. The method of any aspect herein, further comprising: providing a surface comprising a plurality of amplification sites, wherein the amplification sites comprise at least two populations of attached single-stranded capture oligonucleotides having a free 3′ end, and contacting the surface comprising amplification sites with the sequencing library under conditions suitable to produce a plurality of amplification sites that each comprise a clonal population of amplicons from an individual member of the sequencing library.
Aspect 50. The method of any aspect herein, further comprising: providing a surface comprising a plurality of amplification sites, wherein the amplification sites comprise at least two populations of attached single-stranded capture oligonucleotides having a free 3′ end; and contacting the surface comprising amplification sites with the converted double-stranded DNA sequencing library under conditions suitable to produce a plurality of amplification sites that each comprise a clonal population of amplicons from an individual member of the converted double-stranded DNA sequencing library.
Aspect 51. A method of detecting the location of a modified cytosine in a target nucleic acid, the method comprising: (a) contacting target nucleic acids suspected of comprising at least one cytosine and at least one modified cytosine with the altered cytidine deaminase of any aspect herein to produce converted nucleic acids comprising at least one converted cytosine; (b) detecting the at least one converted cytosine in the converted nucleic acids of (a).
Aspect 52. The method of any aspect herein, wherein the altered cytidine deaminase has cytosine-selective deaminase activity, wherein the detecting comprises identifying uracil nucleotides in the converted nucleic acid to determine the location of C nucleotides, 5mC nucleotides, and 5hmC nucleotides in the target nucleic acid.
Aspect 53. The method of any aspect herein, wherein the detecting comprises sequencing the converted nucleic acids.
Aspect 54. The method of any aspect herein, wherein the detecting comprises hybridizing nucleic acid probes to the converted nucleic acids.
Aspect 55. The method of aspect 51, wherein the detecting comprises sequencing the converted nucleic acids, the method further comprising: (c) comparing the sequence of the converted nucleic acids with an untreated reference sequence to determine which cytosines in the target nucleic acids are modified.
Aspect 56. The method of any aspect herein, wherein a predetermined sequence of the untreated reference sequence and a predetermined sequence of the converted nucleic acid are compared.
Aspect 57. The method of any aspect herein, wherein the predetermined sequence comprises a CpG island or a promoter.
Aspect 58. The method of any aspect herein, wherein the detecting comprises hybridizing the converted nucleic acids to the nucleic acid probes, optionally wherein the nucleic acid probes are present on an analyte array, the method further comprising sequencing the hybridized converted nucleic acids.
Aspect 59. The method of any aspect herein, wherein the detecting comprises hybridizing the converted nucleic acids to the nucleic acid probes, the method further comprising amplifying the converted nucleic acid, wherein the nucleic acid probes comprise two primers for amplification of a predetermined sequence, wherein the primers anneal to regions of converted nucleic acids comprising at least one converted cytosine with a greater affinity than to the regions of converted nucleic acids wherein at least one cytosine is not a converted cytosine, wherein the presence of an amplified product is indicative of a modified cytosine in the target nucleic acid.
Aspect 60. The method of any aspect herein, wherein the detecting comprises hybridizing the converted nucleic acids to the nucleic acid probes, the method further comprising cleaving a single stranded DNA (ssDNA) reporter substrate by a CRISPR-based system, wherein the ssDNA reporter substrate comprises a fluorophore and a quencher, wherein the presence of fluorescence is indicative of a modified cytosine in the target nucleic acid.
Aspect 61. The method of any aspect herein, wherein the CRISPR-based system comprises a guide RNA sequence that anneals to a predetermined sequence of a nucleic acid comprising at least one converted cytosine and anneals at lower affinity to the predetermined sequence of the nucleic acid when at least one cytosine is not a converted.
Aspect 62. The method of any aspect herein, wherein the CRISPR-based system comprises CRISPR-Cas12.
Aspect 63. The method of any aspect herein, wherein the detecting comprises hybridizing the converted nucleic acids to the nucleic acid probes, wherein the converted nucleic acids are present in a fixed cell, wherein the nucleic acid probes comprise a fluorescent labeled probe, and wherein the nucleic acid probes anneal to a predetermined sequence of converted nucleic acids comprising at least one converted cytosine with a greater affinity than to the regions of converted nucleic acids wherein at least one cytosine is not a converted cytosine, wherein the presence of cell-associated fluorescence is indicative of a modified cytosine in the target nucleic acid.
Aspect 64. The method of any aspect herein, wherein the untreated reference sequence is a predetermined sequence.
Aspect 65. The method of any aspect herein, wherein the at least one modified cytosine is a 5mC or a 5hmC.
Aspect 66. The method of any aspect herein, further comprising providing the target nucleic acids, wherein the providing comprises preparing single stranded (ss) DNA from a sample comprising the target nucleic acids.
Aspect 67. The method of any aspect herein, wherein the target nucleic acids are genomic DNA or cell free DNA.
Aspect 68. The method of any aspect herein, wherein the genomic DNA is from a single cell or is a mixture from a plurality of cells.
Aspect 69. The method of any aspect herein, wherein the sequencing comprises processing the converted nucleic acids to produce a sequencing library.
Aspect 70. The method of any aspect herein, further comprising: providing a surface comprising a plurality of amplification sites, wherein the amplification sites comprise at least two populations of attached single-stranded capture oligonucleotides having a free 3′ end, and contacting the surface comprising amplification sites with the sequencing library under conditions suitable to produce a plurality of amplification sites that each comprise a clonal population of amplicons from an individual member of the sequencing library.
Aspect 71. The method of any aspect herein, wherein the target nucleic acids are obtained from a subject, wherein the detecting comprises obtaining a pattern of cytosine modification in the converted nucleic acids, the method further comprising comparing the pattern of cytosine modification in the converted nucleic acids with the pattern of cytosine modification in a reference nucleic acid.
Aspect 72. The method of any aspect herein, wherein the subject has or is at risk of having a disease or condition, wherein the reference nucleic acid is from a normal subject.
Aspect 73. The method of any aspect herein, wherein the pattern of cytosine modification is linked in-cis to a coding region that is correlated with a disease or condition.
Aspect 74. The method of any aspect herein, wherein the pattern of cytosine modification is linked in-cis to a coding region, wherein the coding region in the reference nucleic acid is transcriptionally active or transcriptionally inactive, wherein the comparing further comprises determining if the pattern of cytosine modification of the converted nucleic acid indicates the coding region is transcriptionally active or transcriptionally inactive in the subject.
Aspect 75. The method of any aspect herein, wherein transcription of the coding region is correlated with a disease or condition.
Aspect 76. The method of any aspect herein, wherein the subject has the disease or condition and is undergoing treatment for the disease or condition, the method further comprising determining if the treatment is correlated with a change in the pattern of cytosine modification in the subject.
Aspect 77. The method of any aspect herein, wherein the subject previously had the disease or condition, the method further comprising comparing the pattern of cytosine modification in the subject with the pattern of cytosine modification in the subject when the subject had the disease or condition.

### EXAMPLES

The present disclosure is illustrated by the following examples. It is to be understood that the particular examples, materials, amounts, and procedures are to be interpreted broadly in accordance with the scope and spirit of the disclosure as set forth herein and not limiting to the present disclosure.

### Example 1

### Protein Expression and Purification.

Constructs containing coding regions encoding altered cytidine deaminases (ACDs) with substitutions mutations predicted to increase stability were produced. The substitutions mutations were present in a scaffold having Y130A/Y132H/C171A (ScB, SEQ ID NO: 68). Constructs were transformed using BL21(DE3) cells and expressed in 50 mL of autoinduction media composed by terrific broth, 0.2% lactose, and kanamycin. The cultures were incubated at 37° C. for 6 h, and then 16° C. overnight. The cell pellets were harvested by centrifugation, resuspended in BugBuster® Protein Extraction Reagent and proteins were purified using HisPur™ Ni-NTA spin columns. The eluates were desalted into 20 mM Tris, 200 mM NaCl, 1 mM DTT, 5% glycerol, and 0.01% tween, and concentrated to ˜2 mg/mL. Analysis by SDS-PAGE gel revealed the purity to be ˜70-80%.

Tm measurement (FIG. 5A).

The melting temperature (Tₘ) of the constructs was determined by DSF (differential scanning fluorimetry). Samples were diluted to 1 mg/mL in storage buffer (20 mM Tris, 200 mM NaCl, 1 mM DTT, 5% glycerol and 0.01% tween pH 7.5) and measured in duplicate with a temperature range from 25-100 C and a heating rate of 1° C./min. Analysis of the minimum of differential curves allowed determination of the Tm.

LC/MS activity assay with temperature profile (FIG. 5B).

Temperature profiles for 5mC and C conversion were determined for each stability mutant using an LC/MS assay. In a typical assay, 1 μM final concentration of enzyme was incubated with 250 nM methylated substrate at 6 different temperatures (30, 35, 40, 45, 50, and 55° C.) for 60 minutes. Reactions were quenched by heating at 95° C. for 5 minutes. The DNA was purified by desalting using ZYMO Oligo Clean and Concentrator followed by digestion using NEB Nucleoside Digestion Mix at 37° C. overnight. The digested samples were analyzed by LC/MS on an Agilent Triple Quadrupole, and the single nucleotides quantified by MS/MS. The fraction of 5mC or dC converted was determined based on the relative amounts of 5mdC, dC, dT and dU compared to the untreated substrate.

### Summary

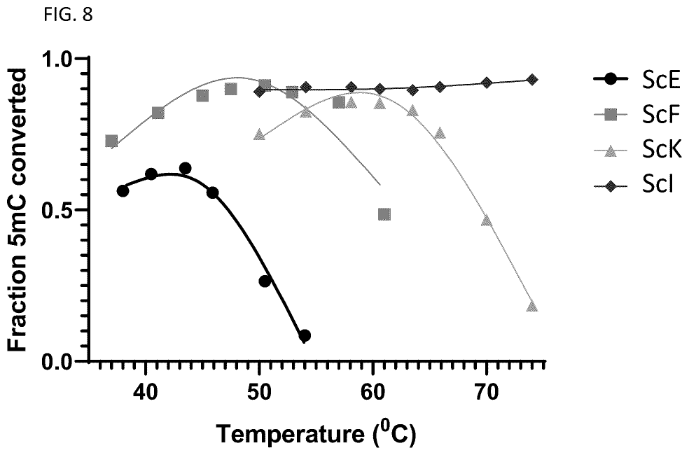

FIGS. 5A and 5B show the results of selected substitution mutations evaluated when present in the scaffold Y130A/Y132H/C171A (ScB, SEQ ID NO:68). Table 5 includes the results shown in FIG. 5A. FIGS. 7 and 8 show the summary of the mutations tested as described in Examples 1, 2, and 3.

### Example 2

### Protein Expression and Purification.

Constructs containing coding regions encoding ACDs with substitutions mutations predicted to increase stability were produced. The substitutions mutations were present in a scaffold having Y130A/Y132H/D133W/R74L/T19Y/C171A (ScE, SEQ ID NO:72). Constructs were transformed in BL21(DE3) and expressed at 37° C. for 6 h, and then incubated overnight at room temperature with autoinduction media (terrific broth supplemented with kanamycin and 0.2% lactose) in a 96 deep well plate. The cell pellets were harvested by centrifugation, and then resuspended in BugBuster® Protein Extraction Reagent. The lysates were purified using Dynabeads® Ni-NTA and eluted in 50 mM Tris, 300 mM NaCl, 250 mM imidazole, 5% glycerol and 0.01% tween using a KingFisher (Thermofisher) automated instrument. Top hits (down selected from initial screen and moved forward to NGS assay) were expressed and purified using spin columns as previously described for Example 1.

Activity-based screen using LC/MS (FIG. 6A).

Purified libraries were screened for activity using an LC/MS assay. Reactions containing 2 μL of enzyme and 250 nM methylated substrate were incubated for 60 minutes in 5 different conditions: reaction buffer (50 mM Tris, 1 mM DTT) at 37 and 45° C., reaction buffer with additives (20% DMSO, 2M betaine) at 37 and 45° C., and reaction buffer with a 10 minute incubation at 45° C. prior to 60 minute incubation at 37° C. Reactions were stopped by incubation at 95° C. for 5 minutes. The DNA was purified by desalting using ZYMO Oligo Clean and Concentrator, followed by digestion using NEB Nucleoside Digestion Mix at 37° C. overnight. The digested samples were analyzed by LC/MS on an Agilent Triple Quadrupole, and the single nucleotides quantified by MS/MS. The fraction of 5mdC or dC converted was determined based on the relative amounts of 5mdC, dC, dT and dU compared to the untreated substrate.

Activity-based screen using sequencing assay with a methylated substrate (FIG. 6B).

Temperature profiles for 5mdC and dC conversion were determined for each stability mutant hit using an NGS assay. For each reaction, 1 μL of enzyme was incubated with 250 nM methylated substrate at 6 different temperatures (30, 35, 40, 45, 50, and 55° C.) overnight. Reactions were stopped by incubation at 95° C. for 5 minutes. Illumina UD Indexes were added by PCR, and the indexed samples pooled and purified using a 1.8×SPRI bead clean-up. The pool was denatured and diluted in Illumina Hyb Buffer 1 to a final concentration of 1 μM. A 30% spike-in of PhiX was added to the library prior to sequencing on an Illumina MiniSeq.

### Summary

FIGS. 6A and 6B show the results of selected substitution mutations evaluated when present in the scaffold Y130A/Y132H/D133W/R74L/T19Y/C171A (ScE, SEQ ID NO:72). The data is also represented in Table 7.

### Example 3

### Protein Expression and Purification.

Constructs containing coding regions encoding ACDs with alterations predicted to increase stability were produced. The substitutions mutations were present in a scaffold having Y130A/Y132H/D133W/R74L/T19Y/C171A/G108C/G188R/G25K/S45W/I17T/A59P/K60R/A6 1-68 (ScK, SEQ ID NO:75). Constructs were transformed in NEB BL21(DE3), grown overnight on LB-kanamycin, at 37° C., and expressed in autoinduction media (terrific broth supplemented with kanamycin and 0.2% lactose) in a 96 deep well plate. Cells were incubated at 37° C. for 6 hours, with shaking, then at 20° C. overnight. The cell pellets were harvested by centrifugation, and then resuspended in BugBuster® Protein Extraction Reagent. The lysates were purified using Dynabeads® Ni-NTA and eluted in 50 mM Tris, 300 mM NaCl, 250 mM imidazole, 5% glycerol and 0.01% tween using a KingFisher (Thermofisher) automated instrument.

Activity-based screen using a sequencing assay.

Purified libraries were screened for activity using an NGS assay with a methylated ultramer substrate. Reactions containing 1 uL of enzyme and 50 nM methylated substrate were incubated for 1-4 h at 65° C. The stability backbone mutant (ScK) was included as a positive control. Reactions were stopped by incubation at 95° C. for 5 minutes. Two PCR steps for annealing were performed to add adapters followed by Illumina UD Indexes for sequencing. Indexed samples were pool and purified using a 1.8X SPRI bead clean-up. The pool was denatured and diluted in Illumina Hyb Buffer 1 to a final concentration of 1 μM. A 30% spike-in of PhiX was added to the library prior to sequencing on an Illumina NextSeq 500.

### Summary

Table 2 and 3 includes the results of other substitution mutations evaluated when present in the scaffold Y130A/Y132H/D133W/R74L/T19Y/C171A/G108C/G188R/G25K/S45W/I17T/A59P/K60R/46 1-68 (ScK, SEQ ID NO:75).

### Example 4

### Protein Expression and Purification.

Constructs containing coding regions encoding ACDs with alterations predicted to increase stability were produced. Constructs were transformed using BL21(DE3) cells and expressed in terrific broth media. Proteins from cell lysates were purified using nickel affinity chromatography, followed by heparin and size exclusion columns. Final proteins (stored in 20 mM Tris, 200 mM NaCl, 1 mM DTT, 5% glycerol, and 0.01% tween) were concentrated to ˜5 mg/mL and confirmed to be ≥95% pure by SDS-PAGE.

Tm measurement (FIG. 7)

The melting temperature (Tₘ) of the constructs was determined by DSF (differential scanning fluorimetry). Samples were diluted to 1 mg/mL in storage buffer (20 mM Tris, 200 mM NaCl, 1 mM DTT, 5% glycerol and 0.01% tween pH 7.5) and measured in duplicate in a temperature range from 25-100° C. with a heating rate of 1° C./min. Analysis of the minimum of differential curves allowed determination of the Tm. Activity-based stability assay using a sequencing assay (FIG. 8).

Temperature profiles for 5mdC and dC conversion were determined for each stability mutant hit using an NGS assay. Enzymes were normalized to 200 nM (final reaction concentration) and incubated with 2.5 ng of a library containing 20% unmethylated lambda, 4% M.SssI CpG methylated pUC19, 76% NA12878. The reactions were incubated for 1 hour at 8 different temperatures. Each reaction was prepared in duplicate with appropriate positive controls. Reactions were stopped by incubation at 95° C. for 10 minutes. Illumina UD Indexes were installed onto samples using PCR. Indexed samples were pooled and purified using a 0.9×SPRI bead clean-up. The pool was denatured and diluted in Illumina Hyb Buffer 1 to a final concentration of 1 pM, and the library was sequenced using Illumina NextSeq 500/550.

### Summary

FIGS. 7 and 8 shows the results of selected alterations (substitution mutations and optional deletions) when present in different scaffolds on stability and fraction of 5mC converted. ScE, SEQ ID NO:72; ScF, SEQ ID NO:74; ScK, SEQ ID NO:75; ScI, SEQ ID NO: 77. FIG. 7 also shows the additive effect of stacking mutations. The last three bars show the addition of A126C and addition of Δ104-105, each resulting in an increase of stability.

### Example 5

Identification of ACDs with Enhanced Preference for Cytosine

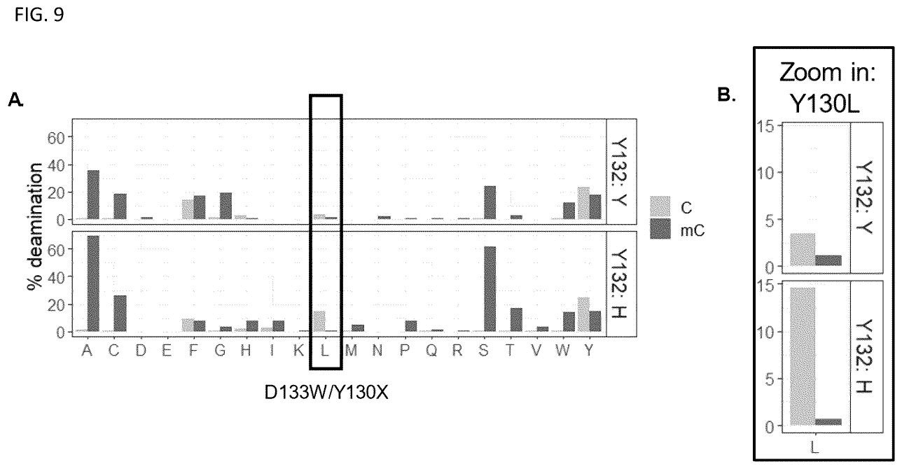

Constructs containing coding regions encoding ACDs with substitutions mutations at specific positions were produced. The substitutions mutations were present in a scaffold having Y130X where X is selected from any amino acid, the substitution of histidine or proline for tyrosine at 132 (Y132H/P) or the wild-type tyrosine at 132, and D133X₁, where X₁ is selected from any amino acid except P or Y (e.g., A, R, N, D, C, E, Q, G, H, I, L, K, M, F, S, T, W, V) in the scaffold of an APOBEC3A, SEQ ID NO:3. Constructs were transformed in NEB BL21(DE3), grown overnight on LB-kanamycin at 37° C., and expressed in autoinduction media (terrific broth supplemented with kanamycin and 0.2% lactose) in a 96 deep well plate. Cells were incubated at 37° C. for 6 hours, with shaking, then at 20° C. overnight. The cell pellets were harvested by centrifugation, and then resuspended in BugBuster® Protein Extraction Reagent. The lysates were purified using Dynabeads® Ni-NTA and eluted in 50 mM Tris, 300 mM NaCl, 250 mM imidazole, 5% glycerol, 10 μM ZnCl2 and 0.01% tween using a KingFisher (Thermofisher) automated instrument. This normally yielded APOBEC3A mutant protein preparations with 80-85% purity, as judged by SDS-PAGE analysis. The results for one round of screening (e.g., using D133W/Y132Y/H and Y130X are shown in FIG. 9. The substitution mutation Y132H was found to enhance the substrate preference of Y130L/D133W for C.

A combinatorial mutagenesis was performed at the 130 and 132 position in an APOBEC3A, SEQ ID NO:3, in the presence of an engineered D133W mutation, and deamination and selectivity activity was assessed by NGS assay. Among 400 mutants cloned, three C-preferring A3A mutants were identified, namely Y130L/Y132H/D133W, Y130F/Y132P/D133W and Y132P/D133W. The Y130A mutation prefers 5mC over C, whereas Y130L prefers C (FIG. 9A). 5mC-specificity of Y130A was shown to be further enhanced by mutating Y132 to H. Interestingly, when Y132H mutation was introduced in Y130L (FIG. 9B), the preference towards C was further enhanced.

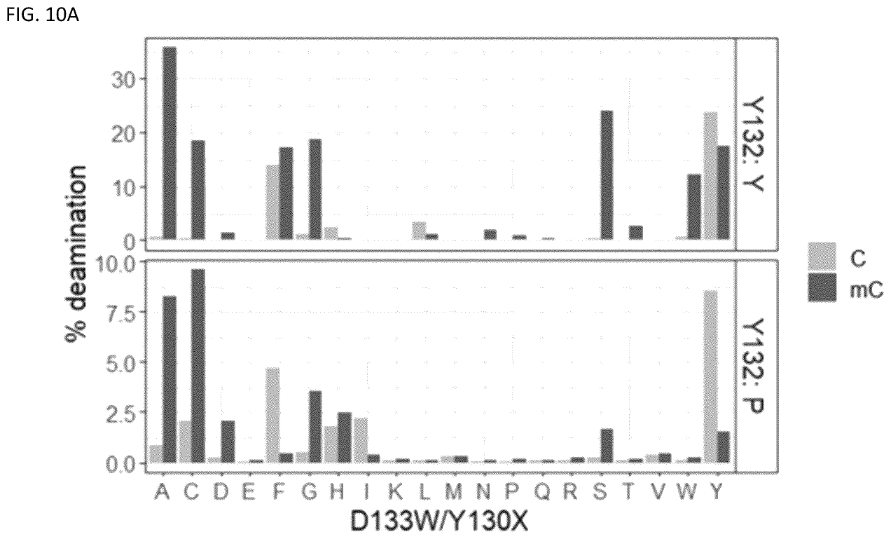

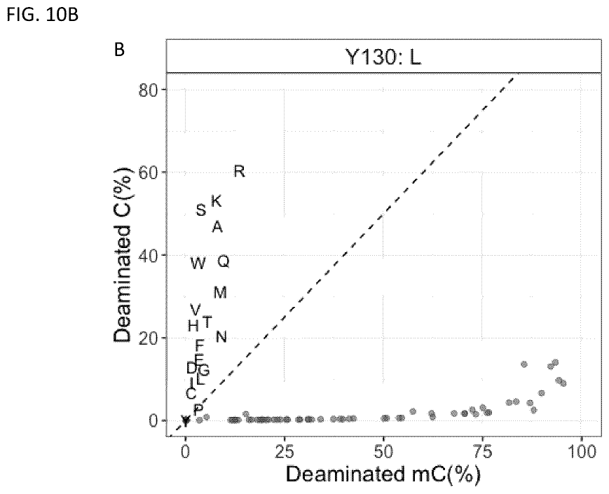

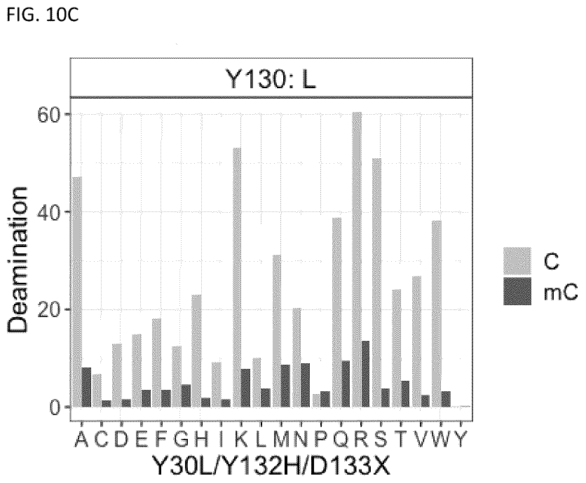

In addition, it was discovered that in the presence of the D133W background mutation, certain variants at position Y130 (Y, F, and I amino acids) conferred C selectivity for the A3A variants when paired with an additional Y132P mutation (FIG. 10). Since the eluate was directly used for deamination reaction and NGS analysis without desalting, the overall activity was compromised. However, selectivity for C or 5mC was still ascertainable by comparing the relative deamination of the respective bases.

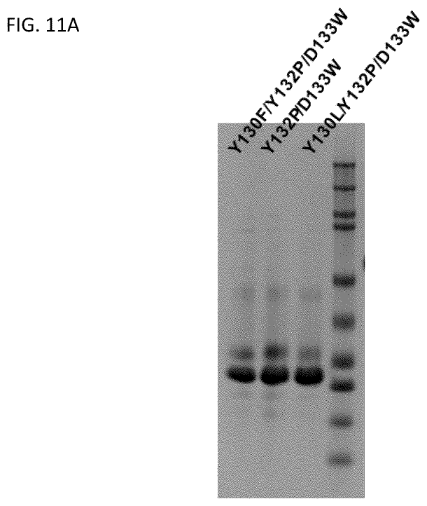

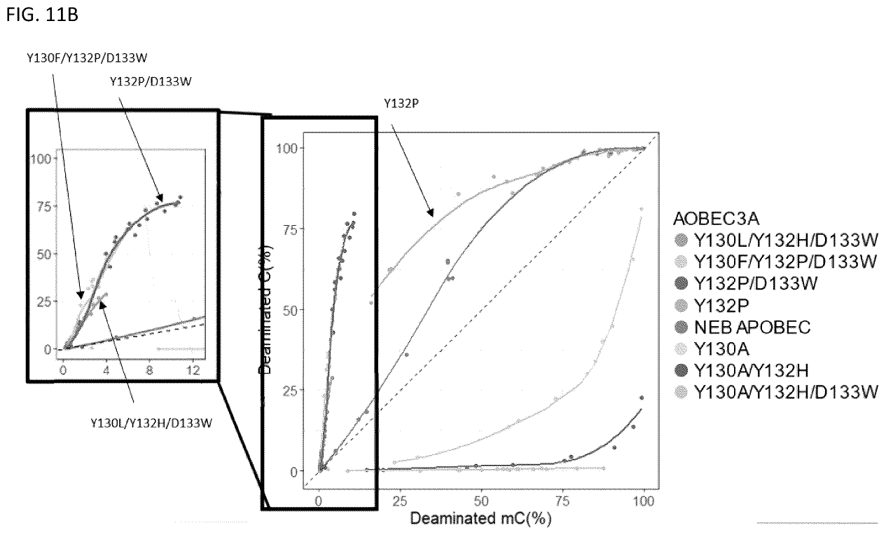

To compare the substrate specificity of these engineered variants against cytidine deaminase NEB A3A present in the NEBNext EM-seq kit (New England Biolabs, MA), deamination reactions were performed at 37° C. for various durations using Ni-NTA purified and desalted proteins (FIG. 11A). The selectivity curve was generated by plotting the percentage of unmethylated C to T conversion (Y-axis) against percentage of 5mC to T conversion (X-axis).

The NEB A3A cytidine deaminase present in the NEBNext EM-seq kit NEB deaminates both C and mC, with slight preference of C over mC (FIG. 11B). The variant with Y132P mutation, has higher selectivity on C, and incorporating D133W mutation further enhanced its preference towards cytosine. The trend lines of three candidates, Y130L/Y132H/D133W, Y130F/Y132P/D133W and Y132P/D133W leaned toward the Y-axis indicating their strong preference toward C over mC deamination.

### Example 6

### Protein Expression and Purification.

Constructs containing coding regions encoding altered cytidine deaminases (ACDs) with one or more substitution alterations predicted to increase selectivity for C over modified cytosines were produced. The substitution alterations were substitution mutations, deletions, and/or insertions. The substitutions mutations were present in a scaffold having an APOBECA3A protein (SEQ ID NO:3) that included the additional substitution mutations Y130A/Y132H/D133W/R74L/T19Y/C171A/G108C/G188R/G25K/S45W/I17T/A59P/K60R/46 1-68 (SEQ ID NO:75). Constructs were transformed in NEB BL21(DE3), grown overnight on LB-kanamycin at 37° C., and expressed in autoinduction media (terrific broth supplemented with kanamycin. 2 mM MgSO₄, 0.2% lactose and 0.05% glucose) in a 96 deep well plate. Cells were incubated at 37° C. for 6 hours, with shaking, then at 20° C. overnight. The cell pellets were harvested by centrifugation, and then resuspended in BugBuster® Protein Extraction Reagent. The lysates were purified using Dynabeads® magnetic beads and eluted in 50 mM Tris, 300 mM NaCl, 250 mM imidazole, 5% glycerol, 10 μM ZnCl₂ and 0.01% v/v tween using a KingFisher (Thermofisher) automated instrument.

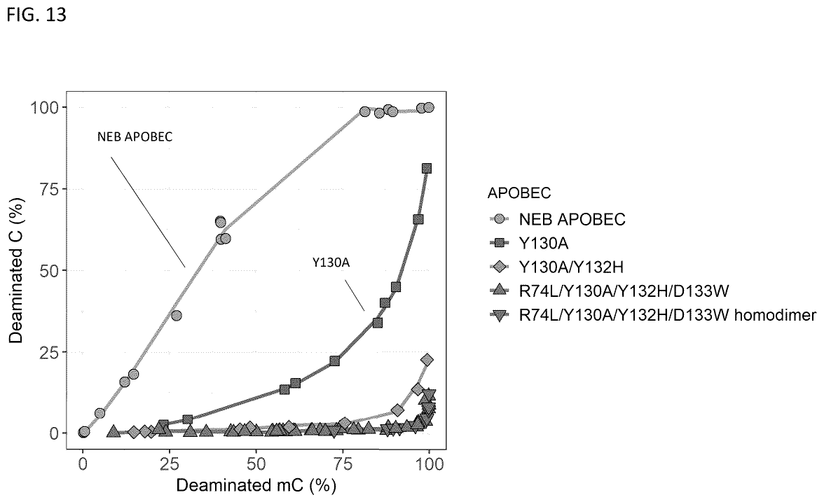

The DNA substrate for experiments in FIG. 10 are shown in FIG. 13 (set A) of International Application Number WO 2023/196572. After the deaminase reaction the deaminated oligo substrates were PCR-amplified, sequenced, and the number of C and 5mC deamination events per read were counted, and expressed as a percentage to the starting number of C and 5mC. The extent of % C and 5mC deamination (Y-axis) is then presented in the form of a bar graph.

### Example 7

### One-Step Enzymatic 5mC Mapping Using C-Specific Cytidine Deaminase Variants

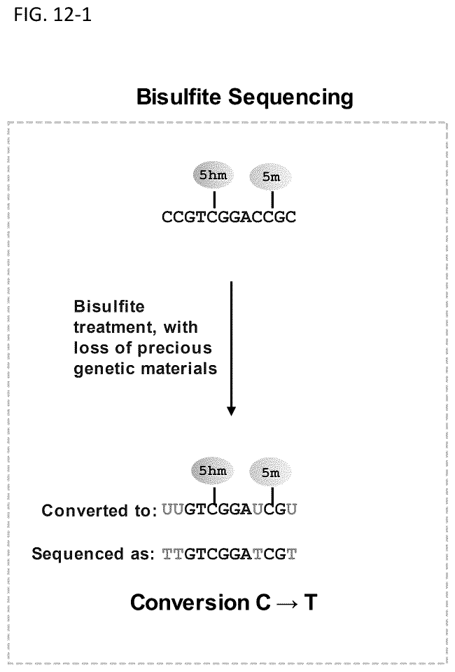

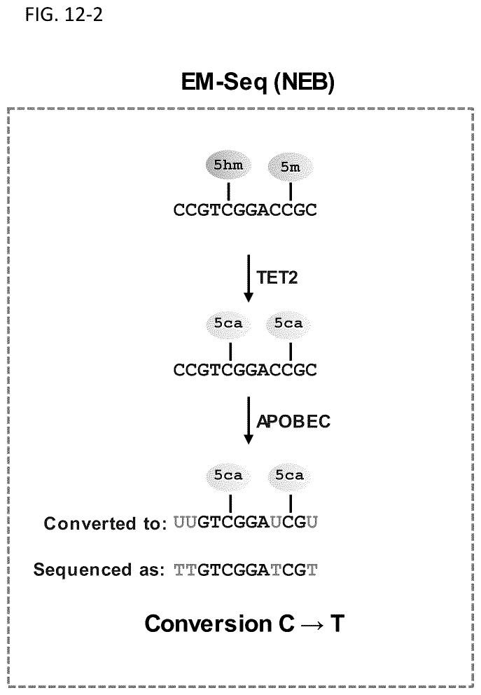

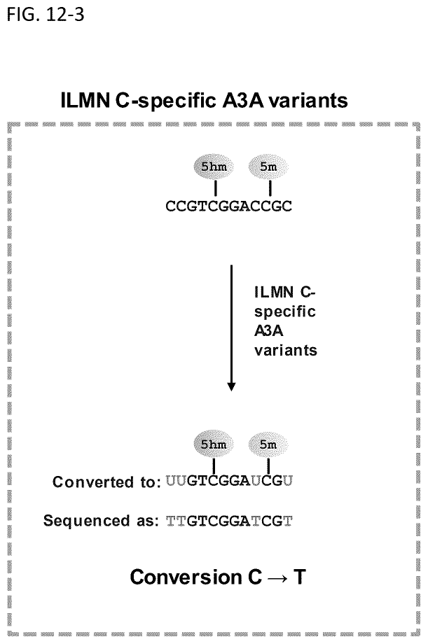

The engineered enzymes Y130L/Y132H/D133W, Y130F/Y132P/D133W, Y132P/D133W, which highly prefer the C over 5mC and 5hmC, are used in single-step enzymatic method for 5mC mapping. In DNA samples treated by these enzymes, C is converted to U, which is identified as T upon sequencing, whereas 5mC remained as C (FIG. 12). By comparing the sequencing reads of treated and untreated samples, methylation level of each C position could be deduced quantitatively. This is a one-step enzymatic alternative to EM-Seq (NEB) which requires two enzymatic steps (C-protection and mC deamination), with more touchpoints and time for 5mC mapping. Similarly, this is an enzymatic and direct alternative to bisulfite sequencing, where the engineered C-selective deaminase is a mild alternative to bisulfite chemical treatment.

### Example 8

### Mutant Cytosine Deaminase Dimers

Examples 8-11 describe the construction and testing of dimers, where the ACD portion of the dimers has the activity of deaminating 5mC at a greater rate than C. It is expected that dimers produced using ACDs having C-preferring activity will have similar characteristics as the dimers produced using 5mC-preferring ACDs but having C-preferring activity.

Dimerizing the mutant cytosine deaminase offers stability and speed of reaction, and do not compromise the 5mC selectivity to its monomeric counterpart (see FIG. 13). Deamination activity of several APOBEC variants were assessed using NGS-based deamination activity assay. The enzymes were incubated with 10 nM DNA ultramer of 126 nucleotides containing 17 C and 16 methylated C, in 10 uL reaction with 50 mM BisTris buffer, at 37° C. for various duration as described in Table 12, followed by one minute of heat inactivation at 95° C. The reactions were then subjected to dual-indexed PCR reaction using uracil tolerant Q5U Hot Start High-Fidelity DNA Polymerase (NEB, M0515). The amplified reactions were pooled together, paired-end sequencing run was performed on a NextSeq 500 system. To quantify the deamination activity, the positions of all unmodified or modified C in every sequencing read were assessed, base changes from C to T were assumed to have been deaminated.

Sequentially introducing mutations into APOBEC, including Y130A/Y132H/D133W on A3A, made the cytidine deaminase highly 5mC selective over unmodified C as shown in the selectivity curves (FIG. 13). The selectivity curves in FIG. 13 also shows the evolution towards 5mC selectivity of A3A mutants we have engineered. This alleviates the difficulties caused by sample degradation and offers better mapping quality by preserving DNA sequence complexity. However, the engineered enzyme became undesirably less stable with subsequent introduction of those abovementioned mutations. FIG. 13 and Table 12 also show that our engineered A3A dimer offer stability and speed, and do not have compromised 5mC selectivity to its monomeric counterpart.

TABLE 12
Various conditions for deamination by NEB APOBEC
and various 5mC selective APOBEC mutants.

| | | | Buffer pH (50 |
|---|---|---|---|
| | Protein amount | Time | mM Bis Tris) |
| NEB APOBEC | 0.01 or 0.001 | 30 seconds | 6.5 |
| | μL/10 μL | to 5 minutes | |
| | reaction | | |
| Y130A | 12 nM or 125 | 7.5 to 120 | 6.5 |
| | nM | minutes | |
| Y130A/Y132H | 12 nM or 125 | 7.5 to 120 | 6.5 |
| | nM | minutes | |
| R74L/Y130A/ | 74 nM to 2000 | 2 to 16 | 7.0 |
| Y132H/D133W | nM | hours | |
| R74L/Y130A/ | 200 nM | 15 minutes | 7.0 |
| Y132H/D133W dimer | | to 4 hours | |

### Example 9

### Exemplary Design of Dimers

It was noted from experimental observation, APOBEC A3A can form dimer in solution although majority population of the A3A exist as monomer in solution. The reason monomer is the major population in vitro is not well understood. Therefore, we tested if expressing two A3A deaminases mutants joined by a linker would encourage dimer formation, and tested if the interactions made between the dimer-dimer interface would then afford stability to the engineered A3A.

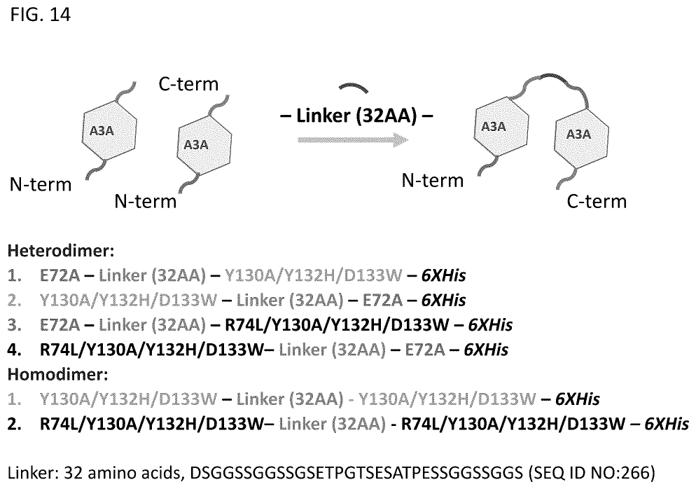

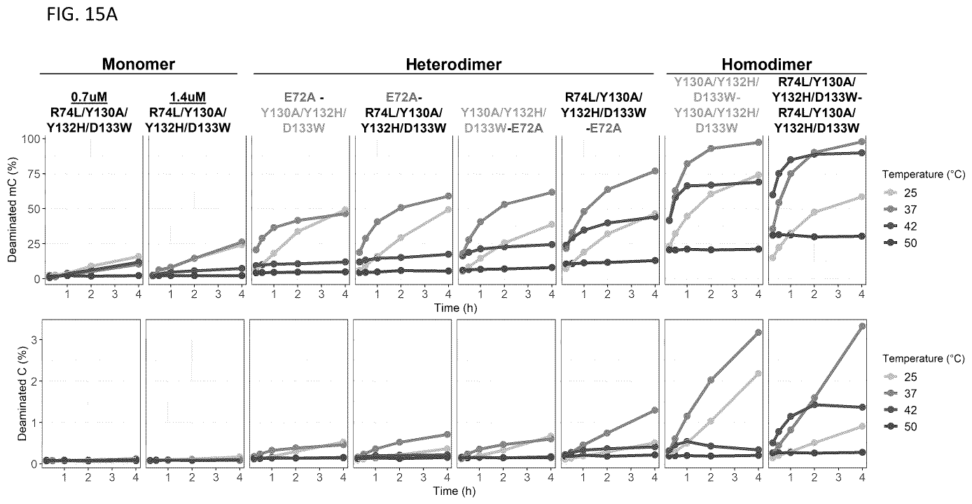

FIG. 14 demonstrates one example of an architecture of engineered dimers, where two A3A deaminases are fused together by a flexible 32-amino acids linker, forming either homodimers or heterodimers. In this example, two homodimeric constructs, A3A (Y130A/Y132H/D133W) and A3A (R74L/Y130A/Y132H/D133W) and four heterodimeric constructs consist of A3A (Y130A/Y132H/D133W) and A3A (R74L/Y130A/Y132H/D133W) with catalytically inactive A3A (E72A) mutant on the C and N-terminals, were made by standard molecular methods.

### Example 10

### Deamination Activity of Dimers

Deamination activity of homodimer and heterodimer altered enzymes were evaluated in NGS-based quantitative deamination assay as described in Example 8, using a DNA ultramer of 126 nucleotides (containing 17 cytosines and 15 5mC interspersed) as substrate. Deamination activity of the enzymes (0.7 μM) were evaluated in NGS-based quantitative deamination assay at various reaction time (0, 15 min, 1 h, 2 h, 4 h) and temperature (25, 37, 42 and 50° C.).

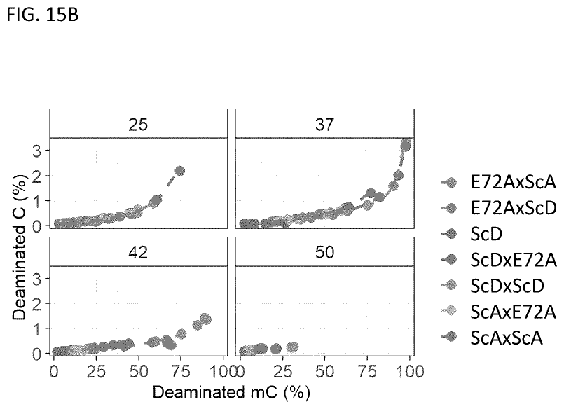

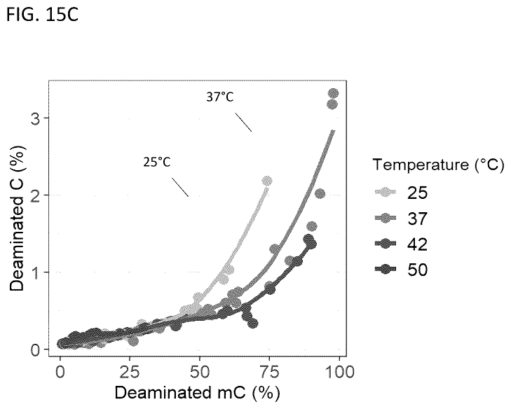

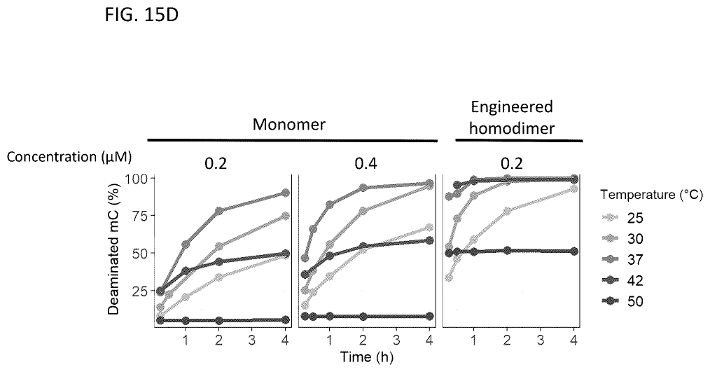

FIG. 15A shows that all six dimeric constructs have improved activity at higher reaction temperature compared to the monomer. The homodimer of R74L/Y130A/Y132H/D133W exhibits higher activity than the homodimer without R74L at 42° C. The introduction of a stability mutation such as R74L helps the dimer to perform deamination at higher temperatures. Additional stabilization mutations appear to have an additive effect on the stability of the dimer. Thus, homodimerization can be combined with other strategies for stabilization (including stabilizing mutations, stabilizing additives, etc.) in combination to create additive and increased stability. This stabilization effect may be applied to the other mutant APOBEC active site variants that have been engineered for selectivity on 5mC, C, or other cytosine adducts such as 5hmC, etc. While all homodimers show an improvement in activity at higher temperatures, the homodimers with two active sites outperform the other four heterodimers which include a single catalytically active domain in terms of rate of reaction and activity at 42° C. Having the active deaminase at the N-terminus helps in the improvement in activity of the dimer. In addition, it was observed that the heterodimers when having catalytically dead A3A (E72A) at C-terminus were more active than those having a catalytically dead A3A (E72A) at the N-terminus.

FIG. 15B shows the relationship of C deamination and 5mC deamination by the A3A (R74L/Y130A/Y132H/D133W) monomer and the six dimers tested at abovementioned temperatures. Data points obtained from the various engineered dimers lie along the same curve as the monomer, indicating that fusing two A3A deaminases together did not impair their preference of deaminating 5mC over unmodified C. When we plotted the deamination data points of the engineered dimers at various temperatures (25° C., 37° C., 42° C. and 50° C.), 42° C. gave the best selectivity (see FIG. 15C). The elevated reaction temperature is favorable for a more unbiased 5mC to T conversion. It was thought that the 5mC is more accessible to the enzyme upon denaturation of DNA secondary structure. Activity at an elevated temperature is a favorable trait offered by the more stable dimer, which is absent in the less stable monomer. This data demonstrates that the dimer exhibited increased conversion and improved selectivity relative to the monomeric ACD.

One protein dimer includes two deaminase domains. Notably, the same deamination efficiency could not be achieved by simply doubling the amount of monomer to match the number of deaminase domains of the dimer (FIG. 15D). For example, 0.4 μM of the monomer saturated at ˜50% 5mC deamination after 2 hours of reaction at 42° C., while 0.2 μM of dimer deaminate 5mC to more than ˜95% after 15 min at 42° C. This indicates that fusion of the two A3A deaminase domains together provides synergy and increases the enzyme stability and activity. Without wishing to be bound by theory, one model for this synergy is that the dimer binding affinity for the monomer in solution is weak, such that the protein concentration is far below the dissociation constant (K_{D}; unknown) of the dimer favoring a majority monomer population. Doubling of the protein concentration is not sufficient to shift this distribution. Fusion of two monomers into one homodimer enforces a high local protein concentration because the two monomers are now fixed together. In addition, preorganization of adjacent monomers by connection in one polypeptide chain reduces the number of possible conformations/orientations of monomers with respect to each other, reducing conformation entropy and favoring dimerization.

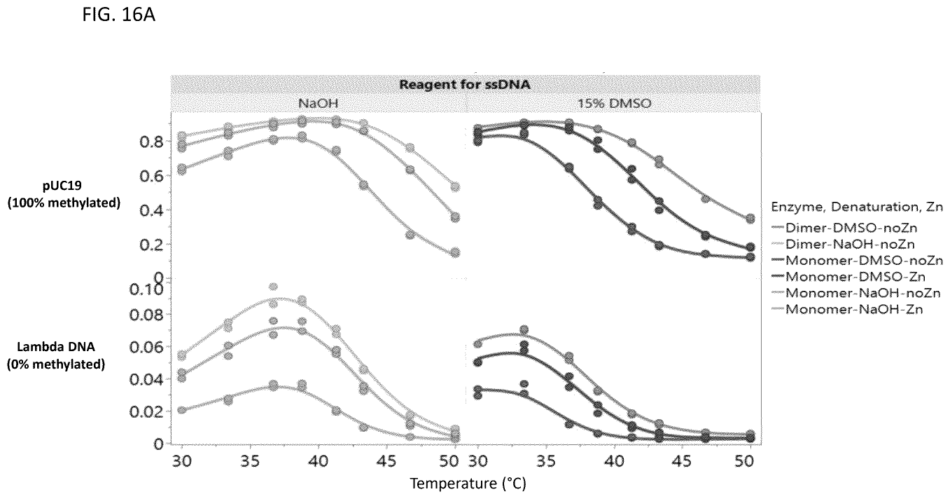

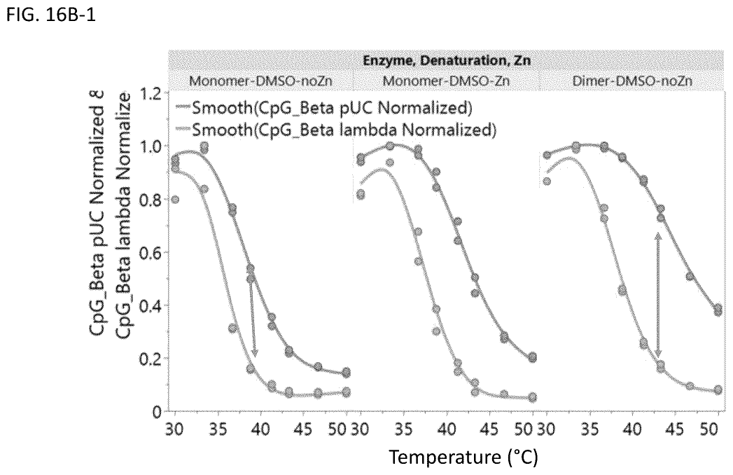

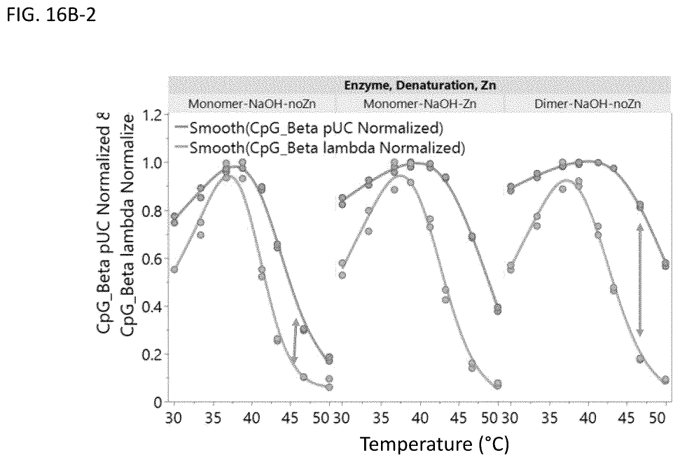

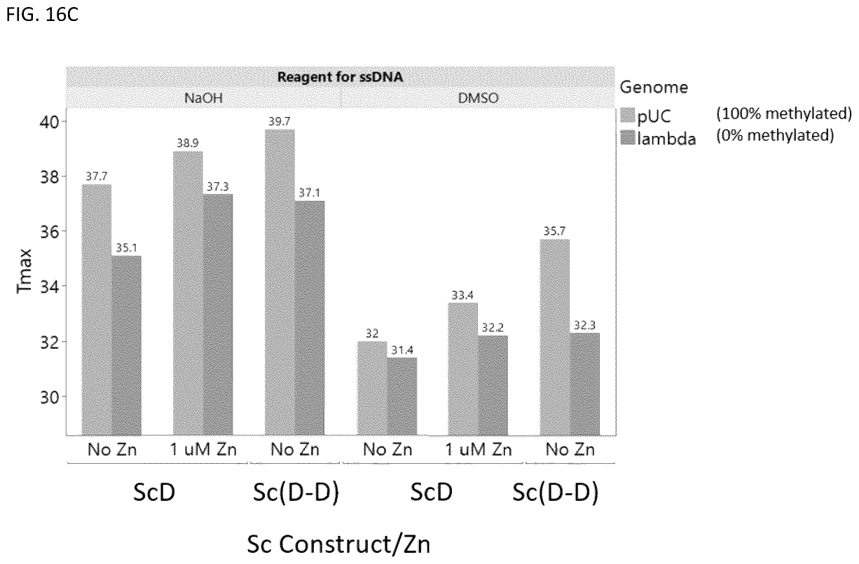

Next, we subjected the altered A3A (R74L/Y130A/Y132H/D133W) homodimer to further assessment on a mixed DNA substrate (a mix of human DNA (NA 12878) with 100% CpG methylated pUC19 and 0% methylated Lambda DNA). The mixed DNA substrate was sheared to 300 nucleotide fragments and a library was prepared using a ligation-based method. Like the ultramer-based deaminase activity assay, the A3A (R74L/Y130A/Y132H/D133W) homodimer exhibited improved stability at temperatures between 40° C.-50° C., whereas the activity of altered A3A (R74L/Y130A/Y132H/D133W) monomer declined rapidly at temperatures greater than 38° C. (FIG. 16A). The homodimer also outperformed the monomer on a DMSO-denatured DNA library, although its activity at higher temperatures was not as high when compared to NaOH-treated DNA.

Although zinc is required for A3A deamination activity at higher temperatures, it has been suggested that the ion may also mediate the dimerization and catalytic activity of A3A. However, supplementing 10 μM zinc chloride salt to the reaction of A3A (R74L/Y130A/Y132H/D133W) monomer did not increase the reaction efficiency to the level of the homodimer (FIG. 16A). Thus, it appears that the dimer confers greater stability than what can be achieved by increasing the concentration of zinc.

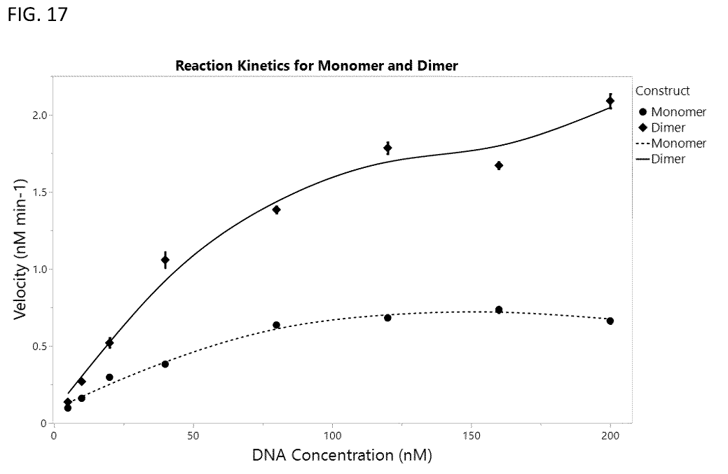

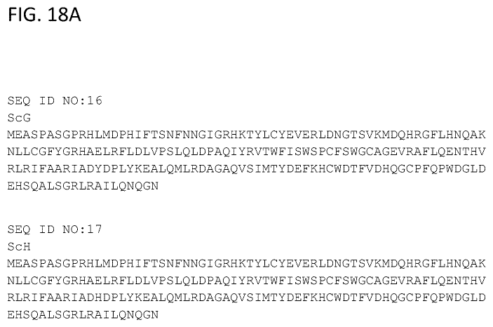

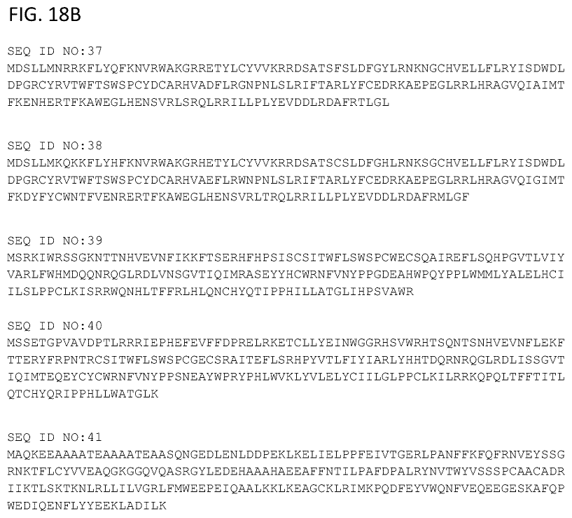

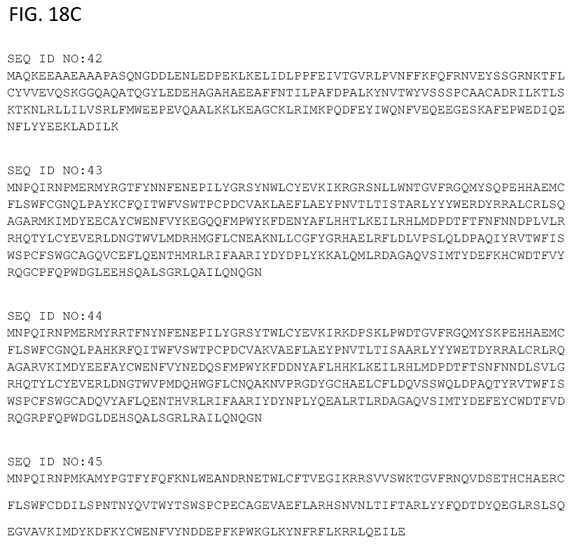

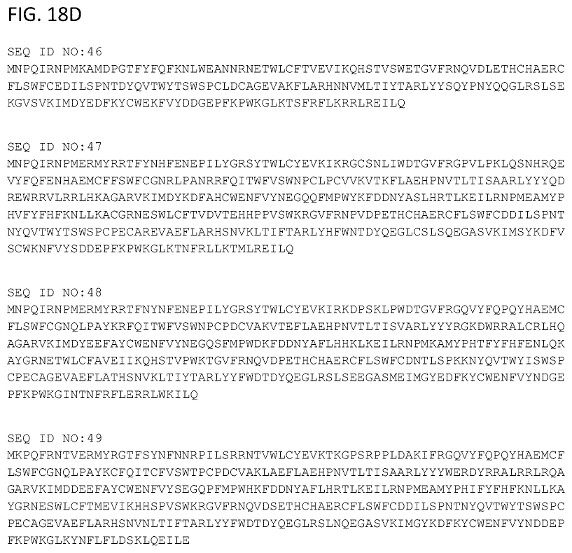

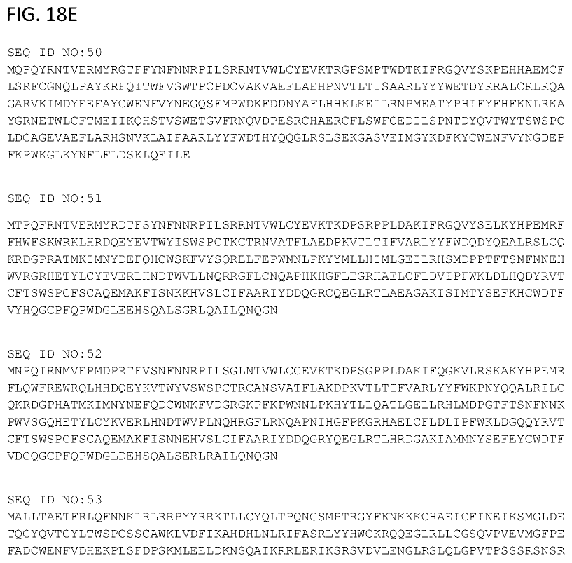

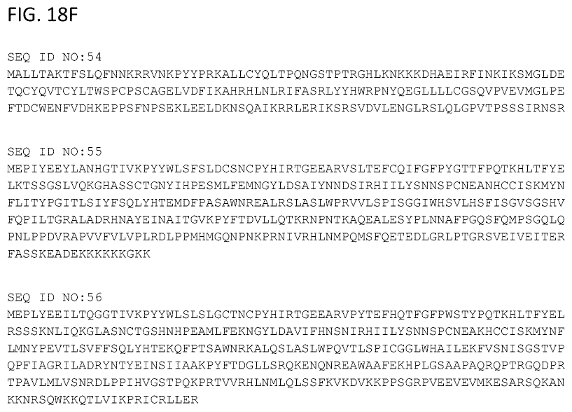

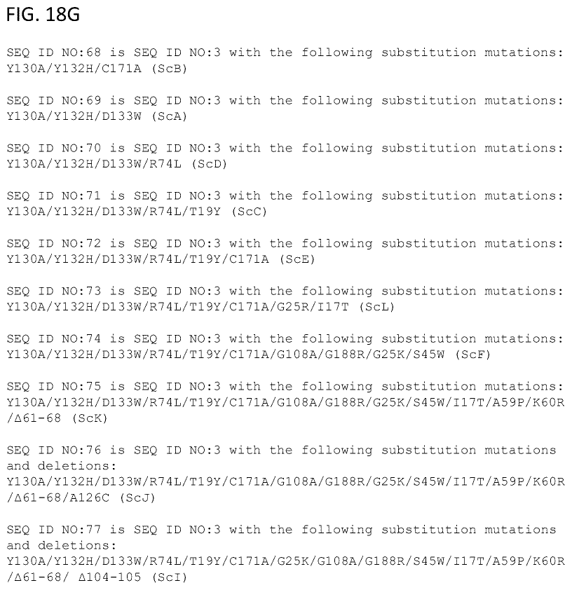

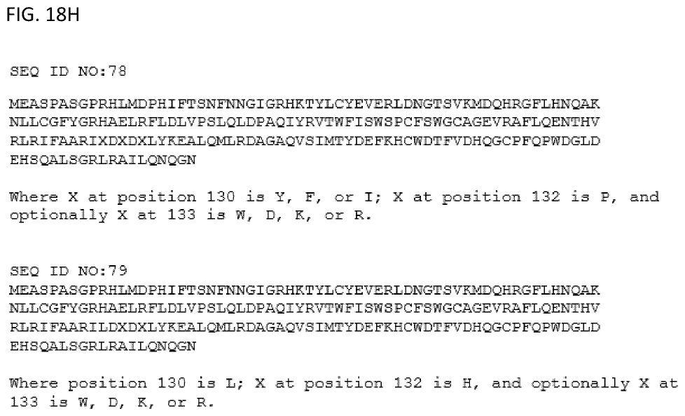

The reaction efficiency of the homodimer and monomer A3 As was measured with increasing concentrations of DNA. At high concentrations of DNA, the dimer exhibited consistently higher velocity (FIG. 17).

A highly selective 5mC engineered deaminase prefers to deaminate methylated pUC19 DNA substrate over non-methylated Lambda DNA substrate when both substrates are present in solution. Thus, to test and compare the selectivity of the deaminase, the altered deaminase was incubated with denatured methylated pUC19 and non-methylated Lambda DNA. The monomeric or homodimeric A3A (R74L/Y130A/Y132H/D133W) were incubated with denatured library prepped DNA containing mCpG methylated pUC19 and unmethylated lambda DNA. The library was denatured with 0.02 N NaOH for 10 minutes at 50° C., and or denatured with DMSO for 10 minutes at 95° C.

To compare how reaction temperatures affect 5mC-selective conversion, 5mC to T conversion level in methylated DNA and C to U conversion level in non-methylated DNA were normalized to 1 by its maximum level (FIG. 16B). In DNA samples denatured with DMSO and NaOH, the monomer had similar reaction profiles on methylated and unmethylated DNA. resulted in differences in the reaction profiles between methylated and unmethylated DNA. Additionally, dimerization resulted in even greater differences between the reaction profiles of methylated and unmethylated DNA. In both the case of Zn added or dimerization, the methylated profile is stabilized at higher temperatures compared to the unmethylated profile. This enables the reaction to be run at higher temperatures where 5mC to T conversion is high and C to U conversion is low, providing a selectivity advantage under these conditions. In each case, the stability of a given construct and formulation can be quantified by Tmax (FIG. 16C). Here, Tmax (° C.) is defined as the temperature at which the enzyme has the highest level of 5mC or C deamination. Tmax is highest for homodimer in both NaOH and DMSO treated denatured DNA samples. This experiment reflects the stability of the dimer towards chemical denaturants.

This example demonstrated that connecting two A3A deaminases with a peptide linker remarkably increases the enzyme performance at higher temperature and in the presence of DNA denaturants, presumably by making the protein dimerization more efficient. Dimerization of any A3A deaminases constructs (wild type or engineered) improves its stability and activity, and this strategy is not limited to just abovementioned engineered A3A deaminases but is applicable to other altered deaminases provided.

### Example 11

### Additional Homodimer/Heterodimer Design

Additional heterodimers are being designed with an inactive N-term monomer (bearing the E72A mutation or other inactivating mutations) connected to an active 5mC-selective altered deaminase as the C-term monomer as shown in Tables 10 and 11. Additional heterodimers include the inverse configuration (active C-terminus) and an inactive N-terminus.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An altered cytidine deaminase (ACD) having C-selective deaminase activity and comprising one or more stability-enhancing alteration, preferably two or more stability-enhancing alterations, wherein conversion of cytosine (C) to uracil (U) by deamination occurs at a greater rate than conversion of 5-methyl cytosine (5mC) to thymidine (T) by deamination, wherein C-selective deaminase comprises:
(a) a substitution mutation at position functionally equivalent to Y130, optionally wherein the substitution is preferably L, F or I, preferably L,
(b) a substitution mutation at position functionally equivalent to Y132, optionally wherein the substitution is preferably H or P;
(c) a substitution mutation at a position functionally equivalent to D133, wherein the substitution is any amino acid except P or Y;
(d) a combination of (a), (b) and (c), wherein the position number designation is functionally equivalent to the position in a wild-type APOBEC3A (SEQ ID NO:3).

2. The ACD of claim 1, wherein the C-selective deaminase activity of the ACD is the result of one or more substitution mutations, deletions, or insertions.

3. (canceled)

4. The ACD of claim 2, wherein (a) the substitution at D133 is W, D, K or R, wherein the at least one amino acid substitution mutation at the position functionally equivalent to Tyr130 is leucine (L), phenylalanine (F), or isoleucine (I), and/or wherein the at least one amino acid substitution mutation at the position functionally equivalent to Tyr132 is histidine or proline, wherein the position number designation is functionally equivalent to the position in a wild-type APOBEC3A (SEQ ID NO:3).

5. The ACD of claim 1, wherein (d) comprises a combination of (a) and (b); (a) and (c); (b) and (c); or (a), (b), and (c).

6.-9. (canceled)

10. The ACD of claim 1, wherein the ACD comprises SEQ ID NO: 80, SEQ ID NO:81, or SEQ ID NO:82, or a sequence having at least 75% or more sequence identity to SEQ ID NO:80, SEQ ID NO:81, or SEQ ID NO:82, preferably at least 90%.

11. The ACD of claim 1, comprising one or more alterations that enhance the selectivity of an ACD for C comprising:
(i) a substitution mutation at a position functionally equivalent to S103X, E138X, Q115X, R28X, Y136X, C108 X, or A112X, X is an amino acid substitution different from the wild-type amino acid at that position,
(ii) a deletion of amino acids at positions functionally equivalent to ΔI26-G27, where the position number designation is functionally equivalent to the position in a wild-type APOBEC3A,
(iii) an insertion at positions functionally equivalent to Σ130-131:X, Σ132-133:X, Σ130-131:X, Σ131-132:X, Σ131-132:X, Σ102-103:X, Σ138-139:X, Σ137-138:X, or Σ134-135:X, where the position number designation is functionally equivalent to the position in a wild-type APOBEC3A (SEQ ID NO:3); or
(iv) a combination of (i) and (ii), (ii) and (iii), (i) and (ii), or (i), (ii), and (iii).

12.-15. (canceled)

16. The ACD of claim 1, wherein the ACD further comprises one or more stability-enhancing alterations, wherein the stability-enhancing alteration is a substitution mutation, a deletion, an insertion, or a combination thereof, wherein the one or more stability-enhancing alterations are selected from (a) substitution mutations at a position functionally equivalent to (a) A112X, A126X, A139X, A148X, A185X, A192X, A59X, A87X, C106X, C161X, C171X, C34X, D145X, D156X, D163X, D167X, D177X, D180X, D41X, D77X, D85X, E109X, E116X, E138X, E157X, E38X, G105X, G108X, G188X, G25X, G27X, H119X, H11X, H16X, H182X, H29X, H51LX, I17X, I26X, I89X, K47X, K60X, L135X, L62X, L78X, M14X, M48X, N117X, N196X, N21X, N42X, P80X, Q115X, Q141X, Q169X, Q184X, R111X, R123X, R189X, R39X, R74X, R91X, S103X, S183X, S187X, S20X, S45X, T118X, T164X, T19X, T31X, T93X, V110X, V79X, L12X, D14X, T19X, G27X, R28X, E38X, F54X, H56X, N57X, Y67X, L73X, D77X, S81X, Y90X, I96X, S97X, C101X, F102X, W104X, C106X, A107X, L114X, V120X, L122X, R128X, Y136X, M142X, A146X, K159X, C161X, L186X, (b) a deletion selected from ΔM1-L12, ΔE36-G53, ΔN61-G68, ΔW104-G105, ΔQ195-N199, or ΔI26-G27, and (c) combinations thereof, wherein the position number designation is functionally equivalent to the position in a wild-type APOBEC3A (SEQ ID NO:3) and X is an amino acid substitution different from the wild-type amino acid at that position.

17.-19. (canceled)

20. The ACD of claim 16, wherein the one or more stability enhancing alterations comprises a substitution mutation at a position functionally equivalent to a stability-enhancing mutation selected from Table 3 and/or a deletion from Table 6, optionally wherein the ACD comprises one or more ancillary substitution mutations.

21.-25. (canceled)

26. The ACD of claim 1, wherein the ACD is based on a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3H subfamily, or the APOBEC4 subfamily, preferably wherein the ACD is APOBEC3A (SEQ ID NO:3).

27.-30. (canceled)

31. A polynucleotide encoding the ACD of claim 1.

32. A composition comprising the ACD of claim 1 and a buffer.

33. The composition of claim 32, wherein the composition further comprises a sample comprising DNA comprising at least one modified cytosine, wherein the modified cytosine is 5-methyl cytosine (5mC), 5-hydroxymethyl cytosine (5hmC), or a combination thereof, wherein the DNA comprises single-stranded DNA, genomic DNA, or cell-free DNA.

34.-38. (canceled)

39. A method comprising:
providing a sample of DNA suspected of comprising double-stranded DNA comprising at least one cytosine, 5-methyl cytosine (5mC), at least one 5-hydroxymethyl cytosine (5hmC), or a combination thereof;
processing the double-stranded DNA to produce a sequencing library;
denaturing the sequencing library to result in a single-stranded DNA;
contacting the single-stranded DNA with an ACD under conditions suitable for conversion of C to U by deamination and 5mC to T by deamination at a greater rate than conversion of 5-hydroxymethyl cytosine (5hmC) to 5-hydroxymethyl uracil (5hmU) by deamination, to result in converted single-stranded DNA,
wherein the altered cytidine deaminase is the ACD of claim 1; and
converting the converted single-stranded DNA to a converted double-stranded DNA sequencing library.

40. The method of claim 39, wherein the processing comprises fragmentation or tagmentation of the double-stranded DNA and addition of a universal sequence to the double-stranded DNA fragments, optionally wherein the universal sequence is part of an adapter added to the double-stranded DNA fragments.

41. (canceled)

42. (canceled)

43. The method of claim 39, wherein the sample is a biological sample, such as cell-free DNA, blood, serum, single cells, isolated nuclei, or a tissue, such as a tumor tissue.

44.-50. (canceled)

51. A method of detecting the location of a modified cytosine in a target nucleic acid, the method comprising:
(a) contacting target nucleic acids suspected of comprising at least one cytosine and at least one modified cytosine with the altered cytidine deaminase of claim 1 to produce converted nucleic acids comprising at least one converted cytosine;
(b) detecting the at least one converted cytosine in the converted nucleic acids of (a).

52. (canceled)

53. The method of claim 51, wherein the detecting comprises sequencing the converted nucleic acids and/or hybridizing nucleic acid probes to the converted nucleic acids.

54.-58. (canceled)

59. The method of claim 53, wherein the detecting comprises hybridizing the converted nucleic acids to the nucleic acid probes, the method further comprising amplifying the converted nucleic acid, wherein the nucleic acid probes comprise two primers for amplification of a predetermined sequence, wherein the primers anneal to regions of converted nucleic acids comprising at least one converted cytosine with a greater affinity than to the regions of converted nucleic acids wherein at least one cytosine is not a converted cytosine, wherein the presence of an amplified product is indicative of a modified cytosine in the target nucleic acid; or
wherein the detecting comprises hybridizing the converted nucleic acids to the nucleic acid probes, the method further comprising cleaving a single stranded DNA (ssDNA) reporter substrate by a CRISPR-based system, wherein the ssDNA reporter substrate comprises a fluorophore and a quencher, wherein the presence of fluorescence is indicative of a modified cytosine in the target nucleic acid.

60.-70. (canceled)

71. The method of claim 51, wherein the target nucleic acids are obtained from a subject, wherein the detecting comprises obtaining a pattern of cytosine modification in the converted nucleic acids, the method further comprising comparing the pattern of cytosine modification in the converted nucleic acids with the pattern of cytosine modification in a reference nucleic acid.

72. The method of claim 71, wherein the subject has or is at risk of having a disease or condition, wherein the reference nucleic acid is from a normal subject, and wherein the pattern of cytosine modification is linked in-cis to a coding region that is correlated with a disease or condition.

73.-77. (canceled)
